(19) United States
(12) Patent Application Publication
(10) Pub. No.: US 2026/0062452 A1
(43) Pub. Date: Mar. 5, 2026

(54) VH3 BINDING POLYPEPTIDES

(71) Applicant: Cytiva BioProcess R&D AB, Uppsala (SE)

(72) Inventors: Mats Arvid Ander, Uppsala (SE); Andreas Lars Magnus Jonsson, Uppsala (SE); Francis Jingxin Hu, Uppsala (SE)

(21) Appl. No.: 18/845,830

(22) PCT Filed: Mar. 14, 2023
(86) PCT No.: PCT/EP2023/056401

§ 371 Date: Sep. 10, 2024

(30) Foreign Application Priority Data
Mar. 14, 2022 (GB) 2203478.9

(51) Int. Cl. C07K 14/31; C07K 1/22

(52) U.S. Cl. CPC C07K 14/31; C07K 1/22

(57) ABSTRACT

The present disclosure relates to a class of engineered polypeptides having a binding affinity for the VH3 region of immunoglobulins and exhibiting desirable alkali clean stability properties. Additionally, the polypeptides exhibit significantly reduced binding affinity for the Fc region of immunoglobulins. The present disclosure also relates to methods for isolating an immunoglobulin or fragment thereof using said polypeptides as well as to related products.

### FIELD OF THE INVENTION

The present disclosure relates to a class of engineered polypeptides having a binding affinity for the VH3 region of immunoglobulins and exhibiting desirable alkali clean stability properties. Additionally, the polypeptides exhibit significantly reduced binding affinity for the Fc region of immunoglobulins. The present disclosure also relates to methods for isolating an immunoglobulin or fragment thereof using said polypeptides as well as to related products.

### BACKGROUND

Immunoglobulins represent the most prevalent biopharmaceutical products in either manufacture or development worldwide. The high commercial demand for and hence value of this particular therapeutic market has led to the emphasis being placed on pharmaceutical companies to maximize the productivity of their manufacturing processes for mAb and/or fragments thereof whilst controlling the associated costs.

Affinity chromatography is used in most cases, as one of the key steps in the purification of these immunoglobulin molecules, such as monoclonal or polyclonal antibodies, or fragments thereof. A particularly interesting class of affinity reagents is proteins capable of specific binding to invariable parts of an immunoglobulin molecule, such interaction being independent of the antigen-binding specificity of the antibody. Such reagents can be widely used for affinity chromatography recovery of immunoglobulins from different samples, such as but not limited to serum or plasma preparations or cell culture derived feed stocks. An example of such a protein is staphylococcal protein A (SpA), containing domains capable of binding to the Fc and Fab portions of IgG immunoglobulins from different species. These domains are commonly denoted as the E-, D-, A-, B- and C-domains.

SpA-based proteins have due to their high affinity and selectivity found a widespread use in the field of biotechnology, e.g. as ligands in affinity chromatography for capture and purification of antibodies as well as for detection or quantification. At present, SpA-based affinity medium is probably the most widely used affinity medium for isolation of monoclonal antibodies and their fragments from different samples including industrial cell culture supernatants. Accordingly, various matrices comprising protein A or protein A-derived ligands are commercially available, for example, in the form of MabSelect™ SuRe, MabSelect™ SuRe LX, MabSelect™ PrismA and HiScreen Fibro™ PrismA from Cytiva™, Uppsala, Sweden.

Certain Protein A and Protein A-derived ligands have binding affinity for both the Fc part of an antibody and for some VH domains of antibodies, in particular VH3. As a result, co-purification of product-related impurities such as half-antibodies and truncated variants may occur and require elution schemes which are complex and/or not sufficiently mild. Additionally, the purification of bispecific antibodies, such as emicizumab, or fragments thereof requires complex elution schemes in order to ensure that only correctly paired bispecific antibodies or fragments thereof are obtained instead of a mixture of correctly and incorrectly paired antibodies and fragments. Thus, there is an unmet need in the field for simplified and reliable isolation methods for antibodies and fragments thereof.

### SUMMARY OF THE INVENTION

It is an object of the present disclosure to provide VH3 binding SpA-derived ligands which exhibit desirable alkali clean stability properties. Such ligands could for example be used in methods for isolating an immunoglobulin and/or fragments thereof.

It is one object of the present disclosure to provide VH3 binding SpA-derived ligands which exhibit no, or significantly reduced affinity for the Fc (fragment crystallizable) region of immunoglobulins.

It is one object of the present disclosure to provide VH3 binding SpA-derived ligands allowing for efficient isolation of immunoglobulins and/or fragments thereof while alleviating the abovementioned and other drawbacks of the prior art.

It is one object to provide VH3 binding SpA-derived ligands enabling mild elution of purified antibodies.

It is a further object to provide VH3 binding SpA-derived ligands which decrease or prevent co-purification of product-related impurities such as half-antibodies and truncated variants.

It is an object of the present disclosure to provide VH3 binding SpA-derived ligands which are alkali clean stabile. Such ligands could for example be used repeatedly for isolation an immunoglobulin and/or fragments thereof.

It is one object to provide VH3 binding SpA-derived ligands which avoid competing interactions and therefore are useful in platform solutions for purification of all VH3 containing products.

It is one object to provide VH3 binding SpA-derived ligands which avoid competing binding interactions from Fc and thus exhibit increased binding capacity for VH3 containing products, in other words VH3 binding SpA-derived ligands which are capable of binding more VH3 containing products.

It is also a further object to provide VH3 binding SpA-derived ligands which avoid competing interactions, e.g. to allow for elution of asymmetric antibodies and/or fragments thereof, such as bispecific or trispecific antibodies and/or fragments thereof.

These and other objects which are evident to the skilled person from the present disclosure are met by different aspects of the invention as claimed in the appended claims and as generally disclosed herein.

Thus, in the first aspect of the disclosure, there is provided a VH3 binding polypeptide derived from a Staphylococcus Protein A (SpA) or any domain thereof, wherein said polypeptide has binding affinity for a VH3 region of trastuzumab and has lower binding affinity for an Fc region of trastuzumab, compared to the binding affinity of SEQ ID NO:88 for the same Fc region and wherein said VH3 binding polypeptide is alkali clean stabile.

As an alternative, the VH3 binding polypeptides as disclosed herein may be evaluated for binding affinity for an Fc region of trastuzumab compared to the affinity of SEQ ID NO:89 for the same Fc region. Thus, as an alternative, there is provided a VH3 binding polypeptide derived from a Staphylococcus Protein A (SpA) or any domain thereof, wherein said polypeptide has binding affinity for a VH3 region of trastuzumab and has lower binding affinity for an Fc region of trastuzumab, compared to the binding affinity of SEQ ID NO:89 for the same Fc region and wherein said VH3 binding polypeptide is alkali clean stabile. Thus, it will be understood that in some embodiments, there is provided a VH3 binding polypeptide derived from a Staphylococcus Protein A (SpA) or any domain thereof as disclosed herein, which has has lower binding affinity for an Fc region of trastuzumab compared to the binding affinity of SEQ ID NO:88 and of SEQ ID NO:89 for the same Fc region.

The skilled person will appreciate that the VH3 binding polypeptide derived from a Staphylococcus Protein A (SpA) or any domain thereof as disclosed herein may for example, but not necessarily, be derived from any one of domains A (SEQ ID NO:92), B (SEQ ID NO:91), C (SEQ ID NO:90), D (SEQ ID NO:93) and E (SEQ ID NO:94) of SpA or derivatives thereof, such as domain Z (SEQ ID NO:89) or variants thereof, such as SEQ ID NO:88. The VH3 binding polypeptide derived from a SpA domain may thus be a derivative, a mutant, a variant or a fragment of an SpA domain as defined above.

The VH3 binding polypeptides of the present disclosure thus are characterized by their retained inherent ability to bind to the VH3 of antibodies/immunoglobulins, in particular to the VH3 region of the antibody trastuzumab. They may also exhibit reduced or abolished binding affinity for the Fc region of the antibody trastuzumab. The skilled person appreciates that similar effect will be observed for antibodies or fragments thereof containing the same or similar VH3 region as trastuzumab as illustrated in the appended Examples.

As used herein, the term VH3 in the context of binding affinity for VH3 refers to the VH3 region of the antibody trastuzumab (trade names Herceptin™ (Roche), Trazimera™ (Pfizer)) which region comprises the amino acid residues in the following positions according to the Kabat numbering system: H15:G; H17:S; H19:R; H57:T; H59:Y; H64:K; H65:G; H66:R; H68:T; H70:S; H81:Q; H82a:N and H82b:S. For avoidance of any doubt, the term “VH3 region of trastuzumab” and the term “VH3” (without any additional specification) are used to refer to the VH3 region of the antibody trastuzumab. The same region VH3 region is also present in the following antibodies: avelumab (trade name Bavencio™), denosumab (trade name Xgeva™), dupilumab (trade name Dupixent™) and emicizumab (trade name Hemlibra™). The VH3 region of trastuzumab is comprised in the fragment antigen-binding region (Fab region) of trastuzumab, which Fab is defined by SEQ ID NO:182 and 183.

As used herein, the term Fc (fragment crystallizable) in the context of “binding affinity for Fc refers to the Fc region of the antibody trastuzumab (trade names Herceptin™ (Roche), Trazimera™ (Pfizer)). The Fc region of trastuzumab corresponds to the Fc region of the antibody subclasses IgG1, IgG2 and IgG4. For avoidance of any doubt, the term “Fc region of trastuzumab” and the term “Fc” (without any additional specification) are used to refer to the Fc region of the antibody trastuzumab. The Fc region of trastuzumab is defined by SEQ ID NO:184.

Additionally, the VH3 binding polypeptides according to the present disclosure are characterized by that their ability to bind to VH3, in other words their affinity for VH3, is not or is only slightly affected by exposure to alkaline conditions, such as for example conditions during which a separation matrix comprising said VH3 binding polypeptides is cleaned. Such conditions may be cleaning with 0.5 M NaOH or KOH or other suitable cleaning liquid known in the art. The skilled person is aware of the conditions conventionally used for cleaning separation matrices and separation columns before repeated use thereof. The term “alkali clean stabile” as used herein, refers to a property of the VH3 binding polypeptides which relates to its affinity for VH3 after exposure to 0.5 M NaOH. A VH3 binding polypeptide as disclosed herein, is considered to be alkali clean stabile if it retains at least 20% of VH3 binding ability, in other words affinity for VH3, after 100 cleaning cycles compared to its VH3 binding ability/affinity for VH3 before the first cleaning cycle. This assessment of alkali clean stability may be as described in appended Example 1.

In particular, as will be discussed in detail below, the VH3 binding polypeptides as disclosed herein, retain a significant part of their VH3 binding properties even after repeated alkali cleaning steps.

In one embodiment of the present aspect, said VH3 binding polypeptide comprises a sequence A, which Sequence A consists of an amino acid sequence selected from i), ii) and iii),

wherein i), ii) and iii) are defined as follows:

i)
(SEQ ID NO: 95)
X₈X₉X₁₀X₁₁AX₁₃X₁₄X₁₅X₁₆X₁₇X₁₈X₁₉PNLX₂₃X₂₄X₂₅X₂₆
RX₂₈X₂₉FIQSLX₃₅X₃₆

wherein, independently from each other,
X₈ is selected from E, D and A;
X₁₅ is selected from E and Q;
X₁₆ is selected from I and V;
X₁₉ is selected from L and M;
X₂₃ is selected from N and T;
X₂₄ is selected from A and E, such as is E;
X₂₅ is selected from D and E, such as is E;
X₃₅ is selected from K, R and H;
X₃₆ is selected from D and H;
ii) an amino acid sequence which has at least 83% identity to a sequence defined by i)
iii) an amino acid sequence which has at least 70% identity to any sequence selected from the group consisting of: residues 8-36 in SEQ ID NO:88, residues 8-36 in SEQ ID NO:89, residues 8-36 in SEQ ID NO:90, residues 8-36 in SEQ ID NO:91, residues 8-36 in SEQ ID NO:92, residues 11-39 in SEQ ID NO:93 and residues 1-29 in SEQ ID NO:94,
   wherein additionally, in each of i), ii) and iii) independently from each other,
X₉ is selected from Q, Y and A;
X₁₀ is selected from Q and Y;
X₁₁ is selected from T, E and R;
X₁₃ is selected from L, E, R, A and Q;
X₁₄ is selected from L, E, R, A, Q and W;
X₁₇ is selected from A, H and L;
X₁₈ is selected from R, L and H;
X₂₆ is selected from Q and S;
X₂₈ is selected from N and A; and
X₂₉ is selected from A and G.

In one embodiment, there is provided a VH3 binding polypeptide wherein said polypeptide has binding affinity for a VH3 region of an antibody, and has low binding affinity for an Fc region of the antibody, and wherein said VH3 binding polypeptide is alkali clean stabile.

The present inventors have surprisingly found that the VH3 binding polypeptides derived from SpA or domains thereof having amino acid residues disclosed herein in at least positions X₉, X₁₀, X₁₁, X₁₃ and X₁₄ and optionally also in positions X₁₇, X₁₈ and X₂₆, at least retain or exhibit improved alkali clean stability properties. Their binding affinity for Fc of trastuzumab is also significantly reduced or abolished. The VH3 binding polypeptides comprising amino acids as defined herein in the above-mentioned positions loose at least a significant part of their affinity for Fc while retaining the affinity for the VH3 region.

In one particular embodiment, said VH3 binding polypeptide has binding affinity for a VH3 region of trastuzumab. In one particular embodiment, said VH3 binding polypeptide has binding affinity for a VH3 region of trastuzumab and has a lower binding affinity for the Fc region of trastuzumab than SEQ ID NO:88 and/or SEQ ID NO:89 has for the same region. As explained above, the VH3 binding polypeptides as disclosed herein may be evaluated for binding affinity for an Fc region of trastuzumab compared to the affinity of SEQ ID NO:88 for the same Fc region. Thus, in one embodiment, there is provided a VH3 binding polypeptide comprising a sequence A, which Sequence A consists of an amino acid sequence selected from i), ii) and iii) as defined above, and has a lower binding affinity for the Fc region of trastuzumab than SEQ ID NO:88 has for the same Fc region. As an alternative, there is provided a VH3 binding polypeptide comprising a sequence A, which Sequence A consists of an amino acid sequence selected from i), ii) and iii) as defined above, and has a lower binding affinity for the Fc region of trastuzumab than SEQ ID NO:89 for the same Fc region. In some embodiments, there is provided a VH3 binding polypeptide as disclosed herein, which has lower binding affinity for an Fc region of trastuzumab compared to the binding affinity of SEQ ID NO:88 and of SEQ ID NO:89 for the same Fc region. Thus, it will be understood by the skilled person that the comparisons of binding affinity for the Fc region of trastuzumab exhibited by the VH3 binding polypeptides of the present disclosure may be made using SEQ ID NO:88 and/or SEQ ID NO:89 as a reference.

The present inventors have surprisingly found that by allowing the amino acid residues in positions corresponding to the positions X₉, X₁₀, X₁₁, X₁₃, X₁₄, X₁₇, X₁₈ and X₂₆ of SEQ ID NO:88 or 89 vary as disclosed herein for VH3 binding polypeptides derived from SpA or domains thereof, VH3 binding polypeptides with desirable properties are obtained. In particular, the binding affinity for Fc of trastuzumab is significantly reduced or abolished, while the VH3 binding polypeptides at least retain or exhibit improved alkali clean stability properties. The VH3 binding polypeptides comprising amino acids as defined herein in the above-mentioned positions loose at least a significant part of their affinity for Fc while retaining the affinity for the VH3 region.

In particular, in one embodiment there is provided a VH3 binding polypeptide as disclosed herein, wherein, independently from each other, in sequence i)
X₈ is selected from E, D and A;
X₁₅ is selected from E and Q;
X₁₆ is selected from I and V;
X₁₉ is selected from L and M;
X₂₄ is E;
X₂₅ is E;
X₂₃ is selected from N and T;
X₃₅ is selected from K, R and H; and
X₃₆ is selected from D and H.

To clarify, it will be appreciated that the % identity in ii) and iii) does not relate to X₉, X₁₀, X₁₁, X₁₃, X₁₄, X₁₇, X₁₈ and X₂₆. Thus, the amino residues in positions X₉, X₁₀, X₁₁, X₁₃, X₁₄, X₁₇, X₁₈ and X₂₆ are as defined above in VH3 binding polypeptides encompassed by the definition according to ii) and iii).

The above definition of a class of sequence related, VH3 binding polypeptides is based on analysis of a number of VH3 binding polypeptide variants, that were selected for the properties of at least maintained affinity for VH3 and alkali clean stability as described in the appended Examples. Also the affinity for Fc was evaluated and VH3 were selected for reduced or abolished affinity for Fc.

As explained herein, the affinity for VH3 and Fc of trastuzumab was evaluated. Trastuzumab, as explained above, may be defined by the amino acid residues in the above-mentioned positions according to the Kabat numbering system. Trastuzumab may also be defined as exhibiting the heavy chain amino acid sequence according to SEQ ID NO:183 and the light chain amino acid sequence according to SEQ ID NO:184.

The VH3 binding polypeptides may comprise the identified sequence A, which corresponds to the region which interacts and binds to Fc of the parent scaffold. This region constitutes two alpha helices, namely helix 1 and helix 2, within a three-helical bundle protein domain. Helix 2 and helix 3 of this three-helical bundle protein constitute a binding surface for interaction with the VH3 region of antibodies.

As the skilled person will realize, the function of any polypeptide is dependent on the tertiary structure of the polypeptide. It is therefore possible to make minor changes to the sequence of amino acids in a polypeptide without affecting the function thereof. Thus, the disclosure encompasses modified variants of the VH3 binding polypeptide. These modified variants are alkali clean stabile. The modified variants are such that their affinity for the VH3 region is at least retained. The variant are such that their affinity for the Fc region is significantly reduced or abolished when compared to that.

The modification of Fc binding affinity identified by the present inventors, is applicable to VH3 binding polypeptides based on or derived from the different three-helical domains of Protein A from Staphylococcus aureus, such as any one of the domains A (SEQ ID NO:92), B (SEQ ID NO:91), C (SEQ ID NO:90), D (SEQ ID NO:93) and E (SEQ ID NO:94), in particular domain B, and derivatives thereof. In particular, said modification may be applicable to VH3 binding polypeptides based on or derived from the three-helical bundle protein domain Z (SEQ ID NO:89), which domain Z is derived from domain B of staphylococcal Protein A, or to the variant SEQ ID NO:88.

Such VH3 binding polypeptides have been described in: WO2003080655, Sjödahl, Eur J Biochem 1977 September; 78(2):471-90, WO2008039141, WO2015005859, U.S. Ser. No. 10/308,690, WO2016079033, WO2017194596, U.S. Pat. No. 9,663,558, WO2016079034, JP2006304633A, WO2007097361, EP1992692A1, WO2012083425, WO2012086660, US20120208234, EP2495254A1, WO2013109302A2, WO2015034000, WO2015034056, CN105481954A, WO2016152946A1, WO2017009421, WO2017014261, WO2018009006, WO2018029158, WO2018029157, WO2019030156, CN109721645A, WO2019093439, WO2020040307 and WO2020157281, which are hereby incorporated by reference.

Thus, the present inventors envision that the amino acid residues in positions X₉, X₁₀, X₁₁, X₁₃, X₁₄, X₁₇, X₁₈ and X₂₆, as disclosed herein may be all or a subset thereof introduced into any one of the previously known VH3 binding polypeptides to achieve the effect of significantly reduced or abolished binding affinity for Fc, while retaining the VH3 binding characteristics and at least retaining the alkali clean stability properties.

Also encompassed by the present disclosure is a VH3 binding polypeptide comprising a Sequence A with at least 83% identity to a polypeptide as defined in i). In some embodiments, the polypeptide may comprise a sequence which is at least 87%, such as at least 90%, such as at least 93%, such as at least 96% identical to a polypeptide as defined in i). For example, it is possible that an amino acid residue belonging to a certain functional group of amino acid residues (e.g. hydrophobic, hydrophilic, polar etc.) could be exchanged for another amino acid residue from the same functional group. In some embodiments, such changes may be made in any position except for X₉, X₁₀, X₁₁, X₁₃, X₁₄, X₁₇, X₁₈ and X₂₆ of the sequence of the VH3 binding polypeptide as disclosed herein. In other embodiments, such changes may be made only in the non-variable positions, also denoted scaffold amino acid residues. In such cases, changes are not allowed in the variable positions, i.e. positions denoted with an “X” in sequence i).

Also encompassed by the present disclosure is a VH3 binding polypeptide comprising a Sequence A which Sequence A has at least 70% identity to any sequence selected from the group consisting of: residues 8-36 in SEQ ID NO:88, residues 8-36 in SEQ ID NO:89, residues 8-36 in SEQ ID NO:90, residues 8-36 in SEQ ID NO:91, residues 8-36 in SEQ ID NO:92, residues 11-39 in SEQ ID NO:93 and residues 1-29 in SEQ ID NO:94. In some embodiments, the polypeptide may comprise a sequence which is at least 74%, such as at least 77%, such as at least 80%, such as at least 83%, such as at least 87%, such as at least 90%, such as at least 93%, such as at least 96%, identical to any sequence selected from the group consisting of: residues 8-36 in SEQ ID NO:88, residues 8-36 in SEQ ID NO:89, residues 8-36 in SEQ ID NO:90, residues 8-36 in SEQ ID NO:91, residues 8-36 in SEQ ID NO:92, residues 11-39 in SEQ ID NO:93 and residues 1-29 in SEQ ID NO:94. In particular embodiments, said sequence is selected from the group consisting of: residues 8-36 in SEQ ID NO:88, residues 8-36 in SEQ ID NO:89, residues 8-36 in SEQ ID NO:90, residues 8-36 in SEQ ID NO:91, residues 8-36 in SEQ ID NO:92, residues 11-39 in SEQ ID NO:93 and residues 1-29 in SEQ ID NO:94. In particular embodiments, said sequence is selected from the group consisting of: residues 8-36 in SEQ ID NO:88 and residues 8-36 in SEQ ID NO:89. In one embodiment, said sequence is SEQ ID NO:88. In one embodiment, said sequence is SEQ ID NO:89.

The term “% identity”, as used throughout the disclosure, may for example be calculated as follows. The query sequence is aligned to the target sequence using the CLUSTAL W algorithm (Thompson et al, Nucleic Acids Research, 22: 4673-4680 (1994)). A comparison is made over the window corresponding to the shortest of the aligned sequences. The shortest of the aligned sequences may in some instances be the target sequence. In other instances, the query sequence may constitute the shortest of the aligned sequences. The amino acid residues at each position are compared and the percentage of positions in the query sequence that have identical correspondences in the target sequence is reported as % identity. In one embodiment of the VH3 binding polypeptides as disclosed herein, said Sequence A fulfills the criteria i) and ii), or the criteria i) and iii), or the criteria ii) and iii). In one embodiment, said Sequence A fulfills all criteria i), ii) and iii).

Encompassed by the present disclosure are also any VH3 binding polypeptides, for example but not limited to polypeptides derived from a Staphylococcus Protein A (SpA) or any domain thereof, which comprise the amino acid residues as defined herein in positions corresponding to the positions X₉, X₁₀, X₁₁, X₁₃, X₁₄, X₁₇, X₁₈ and X₂₆ of SEQ ID NO:88. Thus any VH3 binding polypeptide, independent of its length and presence substitution, insertion mutations or deletion mutations, which comprises the amino acid residues as defined herein in the above-mentioned positions is encompassed herein. The identification of such VH3 binding polypeptides lies within the skills of a person skilled in the art. The person skilled in the art is able to identify such VH3 polypeptides by performing a sequence alignment using for example any common alignment tool in known in the art, such as the standard protein blast (blastp). By choosing the appropriate algorithm parameters, including general parameters such as threshold and word size; scoring parameters such as alignment matrix, gap costs and compositional adjustments; and filters and masks, the skilled person may adjust the properties of the alignment.

In particular, the present disclosure encompasses any VH3 binding polypeptides, for example but not limited to polypeptides derived from a Staphylococcus Protein A (SpA) or any domain thereof, which comprise the amino acid residues as defined herein in positions corresponding to the positions X₉, X₁₀, X₁₁, X₁₃, X₁₄, X₁₇, X₁₈ and X₂₆ of SEQ ID NO:88 and which VH3 binding polypeptides exhibit the properties of affinity for VH3 of trastuzumab compared to SEQ ID NO:88, reduced or abolished affinity for Fc of trastuzumab compared to SEQ ID NO:88 and which are alkali clean stabile. As used herein, the term “positions corresponding to” in this context refers to the positions of the amino acid residues X₉, X₁₀, X₁₁, X₁₃, X₁₄, X₁₇, X₁₈ and X₂₆ in relation to each other within such alignment. It is to be understood that the presence or absence of amino acid residues between the positions of the amino acid residues X₉, X₁₀, X₁₁, X₁₃, X₁₄, X₁₇, X₁₈ and X₂₆ in a VH3 binding polypeptide, which presence or absence does not significantly alter the alpha helical tertiary structure in the region of the VH3 binding polypeptide which encompasses the amino acid residues X₉, X₁₀, X₁₁, X₁₃, X₁₄, X₁₇, X₁₈ and X₂₆ and/or which do not alter the VH3 binding surface comprising said residues, is not to be interpreted as that said positions do not correspond to those of SEQ ID NO:88. On the contrary, any VH3 binding polypeptide which comprises one or more deletion or insertion mutations between the position corresponding to amino acid residues X₉, X₁₀, X₁₁, X₁₃, X₁₄, X₁₇, X₁₈ and X₂₆ in SEQ ID NO:88, which mutation(s) do not significantly alter the alpha helical tertiary structure of the region of the VH3 polypeptide and/or VH3 binding surface comprising said residues, are also encompassed herein.

Thus, in one embodiment there is provided an VH3 binding polypeptide derived from a Staphylococcus Protein A (SpA) or any domain thereof, wherein the positions, which in an alignment with SEQ ID NO:88 corresponds to positions X₉, X₁₀, X₁₁, X₁₃, X₁₄, X₁₇, X₁₈ and X₂₆ in said SEQ ID NO:88, independently of each other are as follows:
X₉ is selected from Q, Y and A;
X₁₀ is selected from Q and Y;
X₁₁ is selected from T, E and R;
X₁₃ is selected from L, E, R, A and Q;
X₁₄ is selected from L, E, R, A, Q and W;
X₁₇ is selected from A, H and L;
X₁₈ is selected from R, L and H;
X₂₆ is selected from Q and S;
X₂₈ is selected from N and A; and
X₂₉ is selected from A and G.

The skilled person will appreciate that said alignment may equally well be made with SEQ ID NO:89. Thus, in another embodiment there is provided an VH3 binding polypeptide derived from a Staphylococcus Protein A (SpA) or any domain thereof, wherein the positions, which in an alignment with SEQ ID NO:89 corresponds to positions X₉, X₁₀, X₁₁, X₁₃, X₁₄, X₁₇, X₁₈ and X₂₆ in said SEQ ID NO:89, independently of each other are as follows:
X₉ is selected from Q, Y and A;
X₁₀ is selected from Q and Y;
X₁₁ is selected from T, E and R;
X₁₃ is selected from L, E, R, A and Q;
X₁₄ is selected from L, E, R, A, Q and W;
X₁₇ is selected from A, H and L;
X₁₈ is selected from R, L and H;
X₂₆ is selected from Q and S;
X₂₈ is selected from N and A; and
X₂₉ is selected from A and G.

In one embodiment according to the first aspect as disclosed herein, X₉ is selected from Q and Y. In another embodiment, X₉ is selected from Q and A. In one embodiment, X₉ is selected from A and Y. In one embodiment, X₉ is A. In one embodiment, X₉ is Y. In one embodiment, X₉ is Q.

In one embodiment, X₁₀ is Q. In one embodiment, X₁₀ is Y.

In one embodiment, X₁₁ is selected from T and E. In one embodiment, X11 is selected from T and R. In one embodiment, X₁₁ is selected from R and E. In one embodiment, X₁₁ is T. In one embodiment, X₁₁ is E. In one embodiment, X₁₁ is R.

In one embodiment, X₁₃ is selected from L, E, R and A; or from L, E, R and Q; or from L, E, A and Q; or from L, R, A and Q; or from E, R, A and Q; in particular selected from L, E, R and A; or from L, E, R and Q; or from L, E, A and Q; or from L, R, A and Q. In one embodiment, X₁₃ is selected from L, E and R; or from L, E and A; or from L, E and Q; or from L, R and A; or from L, R and Q; or from L, A and Q; or from E, R and A; or from E, R and Q; or from E, A and Q; or from R, A and Q; in particular selected from L, E and R; or from L, E and A; or from L, E and Q; or from L, R and A; or from L, R and Q. In one embodiment, X₁₃ is selected from L and E; or from L and R; or from L and A; or from L and Q; or from E and R; or from E and A; or from E and Q; or from R and A; or from R and Q; or from A and Q; in particular from L and E; or from L and R; or from L and A; or from L and Q. In one embodiment, X₁₃ is L. In one embodiment, X₁₃ is A. In one embodiment, X₁₃ is E. In one embodiment, X₁₃ is Q. In one embodiment, X₁₃ is R.

In one embodiment, X₁₄ is selected from A, W, E, L and Q; or from A, W, E, L and R; or from A, W, E, Q and R; or from A, W, L, Q and R; or from A, E, L, Q and R; or from W, E, L, Q and R. In one embodiment, X₁₄ is selected from A, W, E and L; or from A, W, E and Q; or from A, W, E and R; or from A, W, L and Q; or from A, W, L and R; or from A, W, Q and R; or from A, E, L and Q; or from A, E, L and R; or from A, E, Q and R; or from A, L, Q and R; or from W, E, L and Q; or from W, E, L and R; or from W, E, Q and R; or from W, L, Q and R; or from E, L, Q and R. In one embodiment, X₁₄ is selected from A, W and E; or from A, W and L; or from A, W and Q; or from A, W and R; or from A, E and L; or from A, E and Q; or from A, E and R; or from A, L and Q; or from A, L and R; or from A, Q and R; or from W, E and L; or from W, E and Q; or from W, E and R; or from W, L and Q; or from W, L and R; or from W, Q and R; or from E, L and Q; or from E, L and R; or from E, Q and R; or from L, Q and R. In one embodiment, X₁₄ is selected from A and W; or from A and E; or from A and L; or from A and Q; or from A and R; or from W and E; or from W and L; or from W and Q; or from W and R; or from E and L; or from E and Q; or from E and R; or from L and Q; or from L and R; or from Q and R. In one embodiment, X₁₄ is A. In one embodiment, X₁₄ is W. In one embodiment, X₁₄ is E. In one embodiment, X₁₄ is L. In one embodiment, X₁₄ is Q. In one embodiment, X₁₄ is R.

In one embodiment, X₁₇ is selected from A and H. In one embodiment, X₁₇ is selected from A and L. In one embodiment, X₁₇ is selected from H and L. In one embodiment, X₁₇ is A. In one embodiment, X₁₇ is H. In one embodiment, X₁₇ is L.

In one embodiment, X₁₈ is selected from R and L. In one embodiment, X₁₈ is selected from R and H. In one embodiment, X₁₈ is selected from H and L. In one embodiment, X₁₈ is R. In one embodiment, X₁₈ is L. In one embodiment, X₁₈ is H.

In one embodiment, X₂₆ is Q. In one embodiment, X₂₆ is S.

In one embodiment, X₂₈ is N. In one embodiment, X₂₈ is A.

In one embodiment, X₂₉ is A. In one embodiment, X₂₉ is G.

In a more specific embodiment defining a sub-class of VH3 binding polypeptides as disclosed herein, when X₁₄ is A then X₁₀ is Y or X₉X₁₀ is AY. Particular examples of VH3 binding polypeptides wherein X₁₄ is A and X₁₀ is Y are SEQ ID NO:2, 7, 10, 11, 13, 14, 15, 16, 21, 27, 31, 36, 41, 43, 48, 51, 52, 54, 55, 56, 57, 62, 67, 72, 77, 82, 83, 84, 85, 86 and 87. Particular examples of VH3 binding polypeptides wherein X₁₄ is A and X₉X₁₀ is AY are SEQ ID NO:7, 10, 11, 13, 14, 15, 16, 21, 27, 31, 36, 41, 48, 51, 52, 54, 55, 56, 57, 62, 67, 72, 77, 82, 83, 84, 85, 86 and 87. Similarly, by studying the sequences of the VH3 binding polypeptides shown in FIG. 5 it is easy to identify which VH3 binding polypeptides are encompassed by each embodiment disclosed herein.

Other embodiments defining sub-classes of VH3 binding polypeptides as disclosed herein may be as follows here:

In one specific embodiment defining a sub-class of VH3 binding polypeptides as disclosed herein, X₉X₁₀X₁₁ is selected from the group consisting of QQT, QYT, YQT, AQE, AQR and AYR; such as the group consisting of QQT, QYT, AQE, AQR and AYR; such as the group consisting of AQE, AQR and AYR; such as the group consisting of AQR and AYR, such as wherein X₉X₁₀X₁₁ is AYR. In another embodiment, X₁₇X₁₈ is selected from the group consisting of AR, HL and LH; such as the group consisting of AR and HL or the group consisting of AR and LH; such as wherein X₁₇X₁₈ is AR. In one embodiment, X₂₄X₂₅ is EE. In one embodiment, X₂₈X₂₉ is selected from the group consisting of NA, NG, AA and AG; such as the group consisting of NA, NG and AG or the group consisting of NG, AA and AS; such as the group consisting of NG and AG or the group consisting of AG and NA or the group consisting of AG and AA. In one embodiment, X₂₈X₂₉ is AA. In one embodiment, X₂₈X₂₉ is AG. In one embodiment, X₂₈X₂₉ is NG. In one embodiment, X₂₈X₂₉ is NA. In one embodiment, independently of each other X₉X₁₀X₁₁ is selected from the group consisting of AQE, AQR and AYR and X₁₃X₁₄ is selected from the group consisting of LA, LW, AA, AE, AL, AQ, AR, EA, EE, EL, EQ, ER, LA, LE, LL, LQ, LR, QA, QE, QL, QQ, QR, RA, RE, RL, RQ and RR; such as wherein independently of each other X₉X₁₀X₁₁ is selected from the group consisting of AQE, AQR and AYR and X₁₃X₁₄ is selected from the group consisting of LA, AA, AE, AL, AQ, AR, EA, EE, EL, EQ, ER, LA, LE, LL, LQ, LR, QA, QE, QL, QQ, QR, RA, RE, RL, RQ and RR; such as wherein independently of each other X₉X₁₀X₁₁ is selected from the group consisting of AQR and AYR and X₁₃X₁₄ is selected from the group consisting of LA, AA, AE, AL, AQ, AR, EA, EE, EL, EQ, ER, LA, LE, LL, LQ, LR, QA, QE, QL, QQ, QR, RA, RE, RL, RQ and RR; such as wherein X₉X₁₀X₁₁ is AYR and X₁₃X₁₄ is selected from the group consisting of LA, AA, AE, AL, AQ, AR, EA, EE, EL, EQ, ER, LA, LE, LL, LQ, LR, QA, QE, QL, QQ, QR, RA, RE, RL, RQ and RR. In one embodiment, X₉X₁₀X₁₁X₁₃X₁₄ is selected from the group consisting of QQTLA, QYTLA, YQTLA, AQELA, AQRLA, AYRLA and AYRLW; such as the group consisting of QQTLA, QYTLA, AQELA, AQRLA, AYRLA and AYRLW; such as the group consisting of AQELA, AQRLA, AYRLA and AYRLW; such as the group consisting of AQRLA and AYRLA; such as wherein X₉X₁₀X₁₁X₁₃X₁₄ is AYRLA.

In one embodiment, independently of each other X₉X₁₀X₁₁ is selected from the group consisting of AQE, AQR and AYR, X₁₃X₁₄ is selected from the group consisting of LA, AA, AE, AL, AQ, AR, EA, EE, EL, EQ, ER, LA, LE, LL, LQ, LR, QA, QE, QL, QQ, QR, RA, RE, RL, RQ, RR and LW and X₁₇X₁₈ is selected from LH and AR; such as wherein independently of each other X₉X₁₀X₁₁ is selected from the group consisting of AQR and AYR, X₁₃X₁₄ is selected from the group consisting of LA, AA, AE, AL, AQ, AR, EA, EE, EL, EQ, ER, LA, LE, LL, LQ, LR, QA, QE, QL, QQ, QR, RA, RE, RL, RQ, RR and LW and X₁₇X₁₈ is selected from LH and AR; such as wherein independently of each other X₉X₁₀X₁₁ is AYR, X₁₃X₁₄ is selected from the group consisting of LA, AA, AE, AL, AQ, AR, EA, EE, EL, EQ, ER, LA, LE, LL, LQ, LR, QA, QE, QL, QQ, QR, RA, RE, RL, RQ, RR and LW and X₁₇X₁₈ is selected from LH and AR; such as wherein independently of each other X₉X₁₀X₁₁ is AYR, X₁₃X₁₄ is selected from the group consisting of LA, AA, AE, AL, AQ, AR, EA, EE, EL, EQ, ER, LA, LE, LL, LQ, LR, QA, QE, QL, QQ, QR, RA, RE, RL, RQ, RR and LW and X₁₇X₁₈ is AR.

In one embodiment, X₉X₁₀X₁₁X₁₃X₁₄X₁₇X₁ₛ is selected from the group consisting of QQTLALH, QYTLALH, YQTLALH, QQTLAAR, AQELALH, AQRLALH, AYRLALH, AYRLWLH, AYRLWAR, AYRLAAR, AYRLAHL and AYRLWHL; such as the group consising of QQTLALH, QYTLALH, QQTLAAR, AQELALH, AQRLALH, AYRLALH, AYRLWLH, AYRLWAR, AYRLAAR, AYRLAHL and AYRLWHL; such as the group consiting of AQELALH, AQRLALH, AYRLALH, AYRLWAR, AYRLAAR and AYRLAHL; such as the group consisting of, AQRLALH, AYRLALH and AYRLAAR; such as the group consisting of AQRLALH and AYRLAAR; such as wherein X₉X₁₀X₁₁X₁₃X₁₄X₁₇X₁₈ is AYRLAAR. In one embodiment, X₂₄X₂₅X₂₈X₂₉ is selected from the group consisting of EENA, EENG, EEAA and EEAG; such as the group consisting of EENA, EENG and EEAG; such as the group consisting of EENG and EEAG; such as wherein X₂₄X₂₅X₂₈X₂₉ is EEAG.

In one embodiment, X₂₆X₂₈X₂₉ is selected from the group consisting of QNG, QAA, QAG, QNA, and SAG; such as the group consisting of QNG, QAG, QNA and SAG; such as the group consisting of QNG, QAG and SAG and or the group consisting QNG, QAG, QNA or the group consisting of QNG, QNA and SAG; such as the group consisting of QAG and SAG; such as wherein X₂₆X₂₈X₂₉ is QAG or SAG.

In one embodiment, X₂₆X₂₈X₂₉ is selected from the group consisting of QAG and SAG; such as wherein X₂₆X₂₈X₂₉ is QAG or SAG. As described in detail in the experimental section to follow, the selection of VH3 binding polypeptide variants has led to the identification of a number of individual amino acid sequences according to Sequence A as defined herein. These sequences constitute individual embodiments of sequence i) according to this aspect. The sequences of individual amino acid sequences according to Sequence A correspond to amino acid positions 8-36 in SEQ ID NO:1-82 presented in FIG. 5.

Hence, in one embodiment according to the first aspect as disclosed herein, there is provided a VH3 binding polypeptide wherein sequence i) corresponds to the sequence from position 8 to position 36 in a sequence selected from the group consisting of SEQ ID NO:1-82, such as the group consisting of SEQ ID NO:1-40 and 42-81, such as the group consisting of SEQ ID NO:1-15 and 42-56. In one embodiment, sequence i) corresponds to the sequence from position 8 to position 36 in a sequence selected from the group consisting of SEQ ID NO:1-41, such as the group consisting of SEQ ID NO:1-40, such as the group consisting of 1-15. In one embodiment, sequence i) corresponds to the sequence from position 8 to position 36 in a sequence selected from the group consisting of SEQ ID NO:1, 2, 4-43 and 45-82; such as the group consisting of SEQ ID NO:5-7, 9-11, 13-41, 46-48, 50-52 and 54-82; such as the group consisting of SEQ ID NO:6-7, 10, 13-41, 47-48, 51 and 54-82; such as the group consisting of SEQ ID NO:6-7, 10, 13, 15-41, 47-48, 51, 54 and 56-82; such as the group consisting of SEQ ID NO:6, 10, 13, 15-41, 47, 51, 54 and 56-82; such as the group consisting of SEQ ID NO:13, 15-41, 54 and 56-82; such as the group consisting of SEQ ID NO:13, 15-40, 54 and 56-81; such as the group consisting of SEQ ID NO:13, 15, 54 and 56. In one embodiment, sequence i) corresponds to the sequence from position 8 to position 36 in a sequence selected from the group consisting of SEQ ID NO:15-41 and 56-82; such as the group consisting of SEQ ID NO:15-40 and 56-81 or the group consisting of SEQ ID NO:15, 41, 56 and 82. In one embodiment, sequence i) corresponds to the sequence from position 8 to position 36 in a sequence selected from the group consisting of SEQ ID NO:14, 15, 55 and 56. In one embodiment, sequence i) corresponds to the sequence from position 8 to position 36 in a sequence selected from the group consisting of SEQ ID NO:1, 2, 4-41; such as the group consisting of SEQ ID NO:5-7, 9-11, 13-41; such as the group consisting of SEQ ID NO:6-7, 10, 13-41; such as the group consisting of SEQ ID NO:6-7, 10, 13, 15-41; such as the group consisting of SEQ ID NO:6, 10, 13, 15-41; such as the group consisting of SEQ ID NO:13, 15-41; such as the group consisting of SEQ ID NO:13, 15-40; such as the group consisting of SEQ ID NO:13 and 15. In one embodiment, sequence i) corresponds to the sequence from position 8 to position 36 in a sequence selected from the group consisting of SEQ ID NO:15-41; such as the group consisting of SEQ ID NO:15-40 or the group consisting of SEQ ID NO:15 and 41. In one embodiment, sequence i) corresponds to the sequence from position 8 to position 36 in a sequence selected from the group consisting of SEQ ID NO:14 and 15.

In one embodiment, sequence i) corresponds to the sequence from position 8 to position 36 in a sequence selected from the group consisting of SEQ ID NO:42-43 and 45-82; such as the group consisting of SEQ ID NO:46-48, 50-52 and 54-82; such as the group consisting of SEQ ID NO:47-48, 51 and 54-82; such as the group consisting of SEQ ID NO:47-48, 51, 54 and 56-82; such as the group consisting of SEQ ID NO:47, 51, 54 and 56-82; such as the group consisting of SEQ ID NO:54 and 56-82; such as the group consisting of SEQ ID NO:54 and 56-81; such as the group consisting of SEQ ID NO:54 and 56. In one embodiment, sequence i) corresponds to the sequence from position 8 to position 36 in a sequence selected from the group consisting of SEQ ID NO:56-82; such as the group consisting of SEQ ID NO 56-81 or the group consisting of SEQ ID NO:56 and 82. In one embodiment, sequence i) corresponds to the sequence from position 8 to position 36 in a sequence selected from the group consisting of SEQ ID NO:55 and 56.

In one embodiment, sequence i) corresponds to the sequence from position 8 to position 36 in SEQ ID NO:13, SEQ ID NO:14 or SEQ ID NO:15. In one embodiment, sequence i) corresponds to the sequence from position 8 to position 36 in SEQ ID NO:13. In one embodiment, sequence i) corresponds to the sequence from position 8 to position 36 in SEQ ID NO:14. In one embodiment, sequence i) corresponds to the sequence from position 8 to position 36 in SEQ ID NO:15. In one embodiment, sequence i) corresponds to the sequence from position 8 to position 36 in SEQ ID NO:41. In one embodiment, sequence i) corresponds to the sequence from position 8 to position 36 in SEQ ID NO:54, SEQ ID NO:55 or SEQ ID NO:56. In one embodiment, sequence i) corresponds to the sequence from position 8 to position 36 in SEQ ID NO:54. In one embodiment, sequence i) corresponds to the sequence from position 8 to position 36 in SEQ ID NO:55. In one embodiment, sequence i) corresponds to the sequence from position 8 to position 36 in SEQ ID NO:56. In one embodiment, sequence i) corresponds to the sequence from position 8 to position 36 in SEQ ID NO:82.

In some embodiments of the present disclosure, the VH3 binding polypeptides are based on variants of SpA domains wherein the mutations according to the present disclosure have been introduced. Thus, in one particular embodiment, there is provided a VH3 binding polypeptide as disclosed herein, having a sequence selected from group consisting of a sequence corresponding to residues 8-36 in SEQ ID NO:83; a sequence corresponding to residues 8-36 in SEQ ID NO:84; a sequence corresponding to residues 8-36 in SEQ ID NO:85; a sequence corresponding to residues 11-39 in SEQ ID NO:86; and a sequence corresponding to residues 1-29 in SEQ ID NO:87.

In some embodiments of the present disclosure, the polypeptide derived from SpA or Sequence A as defined above “forms part of” a three-helix bundle protein domain. This is understood to mean that the sequence of the SpA derived polypeptide or Sequence A is “inserted” into or “grafted” onto the sequence of the original three-helix bundle domain, such that the grafted sequence replaces a similar structural motif in the original domain. For example, without wishing to be bound by theory, the Sequence A is thought to constitute two of the three helices of a three-helix bundle and can therefore replace such a two-helix motif within any three-helix bundle. As the skilled person will realize, the replacement of two helices of the three-helix bundle domain by the two Sequence A helices has to be performed so as not to affect the basic structure of the polypeptide. That is, the overall folding of the Ca backbone of the polypeptide according to this embodiment of the invention is substantially the same as that of the three-helix bundle protein domain of which it forms a part, e.g. having the same elements of secondary structure in the same order etc. Thus, a Sequence A according to the disclosure “forms part” of a three-helix bundle domain if the polypeptide according to this embodiment has the same fold as the original domain, implying that the basic structural properties are shared, those properties e.g. resulting in similar CD spectra. The skilled person is aware of other parameters that are relevant.

Thus, in one embodiment the SpA derived polypeptide or Sequence A forms part of a three-helix bundle protein domain. In some embodiments, said Sequence A forms, or essentially forms, part of two helices with an interconnecting loop, within said three-helix bundle protein domain. In particular, said three-helix bundle protein domain may be selected from domains from bacterial receptor proteins, in other words from bacterial receptor domains. Non-limiting examples of such domains are the five different three-helical domains of Protein A from Staphylococcus aureus, such as domain A, B, C, D and E, in particular domain B and derivatives thereof and domain C and derivatives thereof. An example of a derivative of said domain B is domain Z. Another example of a derivative of domain C is domain C with the mutation G29A. In one embodiment, said three-helix bundle protein domain is selected from domains of protein A from Staphylococcus aureus or derivatives thereof. In some embodiments, the three-helical bundle protein domain is a variant of domain Z, which is derived from domain B of staphylococcal Protein A. In some embodiments, the three-helical bundle protein domain is a domain C comprising the mutation G29A.

The skilled person will appreciate the that the third helix of the three-helix bundle may be provided in said VH3 binding polypeptides as defined herein and that the amino acid sequence of the third helix may vary without significantly departing from its secondary structure or from significantly altering the secondary structure of the polypeptide comprising Sequence A and the third helix. Thus, in one embodiment, there is provided a VH3 binding polypeptide as defined herein, further comprising a Sequence B arranged C-terminally of said sequence A, which Sequence B consists of an amino acid sequence selected from iv) and v), and wherein iv) and v) are defined as follows:

iv)
(SEQ ID NO: 96)
DPSX₄₀SX₄₂X₄₃X₄₄LX₄₆EAX₄₉X₅₀LNX₅₃X₅₄

wherein, independently from each other,
X₄₀ is selected from Q, T and V;
X₄₂ is selected from A, K, L and T;
X₄₃ is selected from N, E, A, and S;
X₄₄ is selected from L, I and V;
X₄₆ is selected from A, G, S and K, such as from A, S and K;
X₄₉ is selected from K and Q, such as is K;
X₅₀ is selected from K and R;
X₅₃ is selected from D, E and K; and
X₅₄ is selected from A and S;
v) an amino acid sequence which has at least 75% identity to a sequence defined by iv).

In some particular embodiments, Sequence B consists of an amino acid sequence selected from iv) and v), and wherein in iv) independently from each other,
X₄₀ is selected from Q, T and V;
X₄₂ is selected from A, K, L and T;
X₄₃ is selected from N, E, A, and S;
X₄₄ is selected from L, I and V;
X₄₆ is selected from A, S and K;
X₄₉ is K;
X₅₀ is selected from K and R;
X₅₃ is selected from D, E and K; and
X₅₄ is selected from A and S.

Thus in yet another embodiment, there is provided a VH3 binding polypeptide as defined herein, which comprises a binding module sequence C, which Sequence C comprises or consists of Sequence A as defined herein and Sequence B as defined herein, in the following order from the N-terminus to the C-terminus
[Sequence A]-[Sequence B]
   or any amino acid sequence which has at least 70% identity to any sequence selected from the group consisting of: residues 8-54 in SEQ ID NO:88, residues 8-54 in SEQ ID NO:89, residues 8-54 in SEQ ID NO:90, residues 8-54 in SEQ ID NO:91, residues 8-54 in SEQ ID NO:92, residues 11-57 in SEQ ID NO:93 and residues 1-47 in SEQ ID NO:94. Thus, in one embodiment said sequence C is selected from the group consisting of amino acid sequence which has at least 70% identity to any sequence selected from the group consisting of: residues 8-54 in SEQ ID NO:88, residues 8-54 in SEQ ID NO:89, residues 8-54 in SEQ ID NO:90, residues 8-54 in SEQ ID NO:91, residues 8-54 in SEQ ID NO:92, residues 11-57 in SEQ ID NO:93 and residues 1-47 in SEQ ID NO:94. In one embodiment, said sequence C is selected from the group consisting sequences of residues 8-54 in SEQ ID NO:88, residues 8-54 in SEQ ID NO:89, residues 8-54 in SEQ ID NO:90, residues 8-54 in SEQ ID NO:91, residues 8-54 in SEQ ID NO:92, residues 11-57 in SEQ ID NO:93 and residues 1-47 in SEQ ID NO:94; wherein independently of each other,
X₉ is selected from Q, Y and A;
X₁₀ is selected from Q and Y;
X₁₁ is selected from T, E and R;
X₁₃ is selected from L, E, R, A and Q;
X₁₄ is selected from L, E, R, A, Q and W;
X₁₇ is selected from A, H and L;
X₁₈ is selected from R, L and H;
X₂₆ is selected from Q and S;
X₂₈ is selected from N and A; and
X₂₉ is selected from A and G;
   wherein the numbering of the amino acid residues corresponds to their position in SEQ ID NO:88.

In one embodiment, said sequence C has at least 70% identity to any sequence selected from the group consisting of: residues 8-54 in SEQ ID NO:88, residues 8-54 in SEQ ID NO:89, residues 8-54 in SEQ ID NO:90, residues 8-54 in SEQ ID NO:91, residues 8-54 in SEQ ID NO:92, residues 11-57 in SEQ ID NO:93 and residues 1-47 in SEQ ID NO:94, has at least 72%, such as at least 74%, such as at least 76%, such as at least 78%, such as at least 80%, such as at least 82%, such as at least 85%, such as at least 87%, such as at least 89%, such as at least 91%, such as at least 93%, such as at least 85%, such as at least 97%, identity to any sequence selected from the group consisting of: residues 8-54 in SEQ ID NO:88, residues 8-54 in SEQ ID NO:89, residues 8-54 in SEQ ID NO:90, residues 8-54 in SEQ ID NO:91, residues 8-54 in SEQ ID NO:92, residues 11-57 in SEQ ID NO:93 and residues 1-47 in SEQ ID NO:94. As previously explained, the % identity does not apply to the amino acid residues in positions X₉, X₁₀, X₁₁, X₁₃, X₁₄, X₁₇, X₁₈, X₂₆, X₂₈ and X₂₉. The amino acid residues in said positions are as defined above.

As discussed above, in some embodiments where the VH3 binding polypeptides as disclosed herein form part of a three-helix bundle protein domain, they essentially form part of at least the three helices and the interconnecting loop between helix 1 (H1) and helix 2 (H2) (referred to as loop 1 (L1)) and the interconnecting loop between helix 2 (H2) and helix 3 (H3) (referred to as loop 1 (L2)). Herein, binding module Sequence C essentially forms part of at least the three helices and the interconnecting loop between helix 1 and helix 2 (referred to as loop 1 (L1)) and the interconnecting loop between helix 2 and helix 3 (referred to as loop 2 (L2)). It will be appreciated that the length of L1 and L2 may vary, for example by the addition or removal of one or more additional amino acid residues, provided that this variation does not significantly alter the three dimensional structure of polypeptide comprising binding module Sequence C.

As explained above, Sequence B comprises L2 and H3. The skilled person will appreciate that the amino acid sequences of L2 and H3 may be independently combined while maintaining the three-dimensional structure of the VH3 binding polypeptide. Thus, in one embodiment, there is provided a VH3 binding polypeptide as defined herein, which comprises a binding module Sequence C, which Sequence C consists of the sequences [Sequence A], [L2] and [H3] in the following order from the N-terminus to the C-terminus; [Sequence A]-[L2]-[H3], wherein [Sequence A] is as defined herein, and wherein, independently from each other, [L2] is selected from the group consisting of:

(SEQ ID NO: 97)
DPSV;
(SEQ ID NO: 98)
DPSQ;
(SEQ ID NO: 99)
EPSQ;
(SEQ ID NO: 100)
DPST;
(SEQ ID NO: 101)
EPSV;
and
(SEQ ID NO: 102)
DPSL;

and [H3] is selected from the group consisting of:

(SEQ ID NO: 103)
SANLLAEAKKLNDA;
(SEQ ID NO: 104)
SKEILAEAKKLNDA;
(SEQ ID NO:105)
SKAILAEAKKLNDA;
(SEQ ID NO: 106)
SANLLAEAKKLNDA;
(SEQ ID NO: 107)
SANLLAEAKKLNES;
(SEQ ID NO: 108)
STNVLGEAKKLNES;
(SEQ ID NO: 109)
SANVLGEAQKLND S;
(SEQ ID NO: 110)
SLEILCEAKKLNDA;
(SEQ ID NO: 111)
SLEILAEAKKLNDA;
and
(SEQ ID NO: 112)
SKKILKEAKKLNKA

In one embodiment, [L2] is selected from the group consisting of: DPSV (SEQ ID NO:97); DPSQ (SEQ ID NO:98); DPST (SEQ ID NO:100);

and DPSL (SEQ ID NO:102). In one embodiment, [L2] is selected from the group consisting of: DPSV (SEQ ID NO:97); DPSQ (SEQ ID NO:98); and DPSL (SEQ ID NO:102). In one embodiment, [L2] is selected from the group consisting of: DPSV (SEQ ID NO:97) and DPSL (SEQ ID NO:102).

In one embodiment [L2] comprises or consists of DPSV (SEQ ID NO:97). In one embodiment [L2] comprises or consists of DPSQ (SEQ ID NO:98). In one embodiment [L2] comprises or consists of DPSL (SEQ ID NO:102).

In one particular embodiment, [H3] is selected from the group consisting of SANLLAEAKKLNDA (SEQ ID NO:103); SKEILAEAKKLNDA (SEQ ID NO:104); SKAILAEAKKLNDA (SEQ ID NO:105); SANLLAEAKKLNDA (SEQ ID NO:106); SANLLAEAKKLNES (SEQ ID NO:107); STNVLGEAKKLNES (SEQ ID NO:108) and SANVLGEAQKLNDS (SEQ ID NO:109). In one embodiment, [H3] is selected from the group consisting of SANLLAEAKKLNDA (SEQ ID NO:103), SKEILAEAKKLNDA (SEQ ID NO:104), and SKAILAEAKKLNDA (SEQ ID NO:105), such as the group consisting of SKEILAEAKKLNDA (SEQ ID NO:104) and SKAILAEAKKLNDA (SEQ ID NO:105), or the group consisting of SANLLAEAKKLNDA (SEQ ID NO:103) and SKAILAEAKKLNDA (SEQ ID NO:105), or the group consisting of SANLLAEAKKLNDA (SEQ ID NO:103) and SKEILAEAKKLNDA (SEQ ID NO:104).

In one embodiment, [H3] comprises or consists of SANLLAEAKKLNDA (SEQ ID NO:103). In one embodiment, [H3] comprises or consists of SKEILAEAKKLNDA (SEQ ID NO:104). In one embodiment, [H3] comprises or consists of SKAILAEAKKLNDA (SEQ ID NO:105).

In one embodiment, there is provided a VH3 binding polypeptide as disclosed herein, which comprises a binding module Sequence C selected from the group consisting of:

(SEQ ID NO: 113)
[Sequence A]-DPSQSANLLAEAKKLNDA;
(SEQ ID NO: 114)
[Sequence A]-DPSVSKEILAEAKKLNDA;
(SEQ ID NO: 115)
[Sequence A]-DPSVSKAILAEAKKLNDA;
(SEQ ID NO: 116)
[Sequence A]-DPSQSANLLAEAKKLNES;
(SEQ ID NO: 117)
[Sequence A]-DPSVSLEILGEAKKLNDA;
(SEQ ID NO: 118)
[Sequence A]-DPSVSLEILCEAKKLNDA;
(SEQ ID NO: 119)
[Sequence A]-DPSVSLEILAEAKKLNDA;
(SEQ ID NO: 120)
[Sequence A]-DPSVSLALLAEAKKLNDA;
(SEQ ID NO: 121)
[Sequence A]-DPSQSANLLSEAKKLNES;
(SEQ ID NO: 122)
[Sequence A]-DPSQSTNVLGEAKKLNES;
and
(SEQ ID NO: 123)
[Sequence A]-DPSQSANVLGEAQKLNDS;

wherein [Sequence A] is as defined as described herein. In particular embodiments, there is provided a VH3 binding polypeptide as disclosed herein, which comprises a binding module Sequence C selected from the group consisting of:

(SEQ ID NO: 113)
[Sequence A]-DPSQSANLLAEAKKLNDA;
(SEQ ID NO: 114)
[Sequence A]-DPSVSKEILAEAKKLNDA;
(SEQ ID NO: 115)
[Sequence A]-DPSVSKAILAEAKKLNDA;
(SEQ ID NO: 116)
[Sequence A]-DPSQSANLLAEAKKLNES;
(SEQ ID NO: 117)
[Sequence A]-DPSVSLEILGEAKKLNDA;
(SEQ ID NO: 118)
[Sequence A]-DPSVSLEILCEAKKLNDA;
(SEQ ID NO: 119)
[Sequence A]-DPSVSLEILAEAKKLNDA;
(SEQ ID NO: 120)
[Sequence A]-DPSVSLALLAEAKKLNDA;
and
(SEQ ID NO: 121)
[Sequence A]-DPSQSANLLSEAKKLNES;

such as the group consisting of:

(SEQ ID NO: 113)
[Sequence A]-DPSQSANLLAEAKKLNDA;
(SEQ ID NO: 114)
[Sequence A]-DPSVSKEILAEAKKLNDA;
(SEQ ID NO: 115)
[Sequence A]-DPSVSKAILAEAKKLNDA;
(SEQ ID NO: 116)
[Sequence A]-DPSQSANLLAEAKKLNES;
(SEQ ID NO: 117)
[Sequence A]-DPSVSLEILGEAKKLNDA;
(SEQ ID NO: 118)
[Sequence A]-DPSVSLEILCEAKKLNDA;
(SEQ ID NO: 119)
[Sequence A]-DPSVSLEILAEAKKLNDA;
and
(SEQ ID NO: 120)
[Sequence A]-DPSVSLALLAEAKKLNDA;

such as the group consisting of:

(SEQ ID NO: 113)
[Sequence A]-DPSQSANLLAEAKKLNDA;
(SEQ ID NO: 114)
[Sequence A]-DPSVSKEILAEAKKLNDA;
(SEQ ID NO: 115)
[Sequence A]-DPSVSKAILAEAKKLNDA;
and
(SEQ ID NO: 116)
[Sequence A]-DPSQSANLLAEAKKLNES;

such as the group consisting of:

(SEQ ID NO: 113)
[Sequence A]-DPSQSANLLAEAKKLNDA;
(SEQ ID NO: 114)
[Sequence A]-DPSVSKEILAEAKKLNDA;
and
(SEQ ID NO: 115)
[Sequence A]-DPSVSKAILAEAKKLNDA;

such as the group consisting of:

(SEQ ID NO: 113)
[Sequence A]-DPSQSANLLAEAKKLNDA;
and
(SEQ ID NO: 115)
[Sequence A]-DPSVSKAILAEAKKLNDA

In one particular embodiment, said binding module Sequence C comprises or consists of [Sequence A]-DPSQSANLLAEAKKLNDA (SEQ ID NO:113). In one particular embodiment, said binding module Sequence C comprises or consists of [Sequence A]-DPSVSKAILAEAKKLNDA (SEQ ID NO:115).

In another embodiment, there is provided a VH3 binding polypeptide comprising or consisting of a binding module Sequence C selected from the group consisting of:

vi)
(SEQ ID NO: 113)
[Sequence A]-DPSQSANLLAEAKKLNDA;

wherein [Sequence A] is as defined herein; and
vii) an amino acid sequence which has at least 85% identity to the sequence defined in vi).

As discussed above, polypeptides comprising minor changes as compared to the above amino acid sequences without largely affecting the tertiary structure and the function thereof also fall within the scope of the present disclosure. Thus, in some embodiments, the VH3 binding polypeptides comprising a binding module Sequence C as defined above may for example have a sequence which is at least 87%, such as at least 89%, such as at least 91%, such as at least 93%, such as at least 95%, such as at least 97% identical to a sequence defined by vi).

In another embodiment, there is provided a VH3 binding polypeptide comprising or consisting of a binding module Sequence C selected from the group consisting of:

viii)
(SEQ ID NO: 115)
[Sequence A]-DPSVSKAILAEAKKLNDA

wherein [Sequence A] is as defined herein; and
ix) an amino acid sequence which has at least 85% identity to the sequence defined in viii).

In another embodiment, there is provided a VH3 binding polypeptide comprising or consisting of a binding module Sequence C selected from the group consisting of:

x)
(SEQ ID NO: 114)
[Sequence A]-DPSVSKEILAEAKKLNDA

wherein [Sequence A] is as defined herein and
xi) an amino acid sequence which has at least 85% identity to the sequence defined in x).

As discussed above, polypeptides comprising minor changes as compared to the above amino acid sequences without largely affecting the tertiary structure and the function thereof also fall within the scope of the present disclosure. Thus, in some embodiments, the VH3 binding polypeptides comprising a binding module Sequence C as defined above may for example have a sequence which is at least 87%, such as at least 89%, such as at least 91%, such as at least 93%, such as at least 95%, such as at least 97% identical to a sequence defined by any one of viii) and x).

As described in detail in the experimental section to follow, the selection of VH3 binding polypeptide variants has led to the identification of a number of individual amino acid sequences and said sequences, also referred to herein as binding module Sequences C, which constitute individual embodiments of any one of sequences vi and viii) corresponding to the sequence from position 8 to position 54 in a sequence selected from the group consisting of SEQ ID NO:1-82, such as the group consisting of SEQ ID NO:1-40 and 42-81, such as the group consisting of SEQ ID NO:1-15 and 42-56 presented in FIG. 5. Hence, the sequences of individual amino acid sequences according to Sequence C correspond to amino acid positions 8 to position 54 in a sequence selected from the group consisting of SEQ ID NO:1-82 presented in FIG. 5.

Hence, in one embodiment according to the first aspect as disclosed herein, there is provided a VH3 binding polypeptide wherein sequence vi) or viii) corresponds to the sequence from position 8 to position 54 in a sequence selected from the group consisting of SEQ ID NO:1-82, such as the group consisting of SEQ ID NO:1-40 and 42-81, such as the group consisting of SEQ ID NO:1-15 and 42-56. In one embodiment there is provided a VH3 binding polypeptide wherein sequence viii) corresponds to the sequence from position 8 to position 54 in a sequence selected from the group consisting of SEQ ID NO:1-41, such as the group consisting of SEQ ID NO:1-40, such as the group consisting of SEQ ID NO:1-15. In one embodiment there is provided a VH3 binding polypeptide wherein sequence vi) or viii) corresponds to the sequence from position 8 to position 54 in a sequence selected from the group consisting of SEQ ID NO:1, 2, 4-43 and 45-82; such as the group consisting of SEQ ID NO:5-7, 9-11, 13-41, 46-48, 50-52 and 54-82; such as the group consisting of SEQ ID NO:6-7, 10, 13-41, 47-48, 51 and 54-82; such as the group consisting of SEQ ID NO:6-7, 10, 13, 15-41, 47-48, 51, 54 and 56-82; such as the group consisting of SEQ ID NO:6, 10, 13, 15-41, 47, 51, 54 and 56-82; such as the group consisting of SEQ ID NO:13, 15-41, 54 and 56-82; such as the group consisting of SEQ ID NO:13, 15-40, 54 and 56-81; such as the group consisting of SEQ ID NO:13, 15, 54 and 56. In one embodiment there is provided a VH3 binding polypeptide wherein sequence vi) or viii) corresponds to the sequence from position 8 to position 54 in a sequence selected from the group consisting of SEQ ID NO:15-41 and 56-82; such as the group consisting of SEQ ID NO:15-40 and 56-81 or the group consisting of SEQ ID NO:15, 41, 56 and 82. In one embodiment there is provided a VH3 binding polypeptide wherein sequence vi) or viii) corresponds to the sequence from position 8 to position 54 in a sequence selected from the group consisting of SEQ ID NO:14, 15, 55 and 56. In one embodiment there is provided a VH3 binding polypeptide wherein sequence viii) corresponds to the sequence from position 8 to position 54 in a sequence selected from the group consisting of SEQ ID NO:1, 2, 4-41; such as the group consisting of SEQ ID NO:5-7, 9-11, 13-41; such as the group consisting of SEQ ID NO:6-7, 10, 13-41; such as the group consisting of SEQ ID NO:6-7, 10, 13, 15-41; such as the group consisting of SEQ ID NO:6, 10, 13, 15-41; such as the group consisting of SEQ ID NO:13, 15-41; such as the group consisting of SEQ ID NO:13, 15-40; such as the group consisting of SEQ ID NO:13 and 15. In one embodiment there is provided a VH3 binding polypeptide wherein sequence viii) corresponds to the sequence from position 8 to position 54 in a sequence selected from the group consisting of SEQ ID NO:15-41; such as the group consisting of SEQ ID NO:15-40 or the group consisting of SEQ ID NO:15 and 41. In one embodiment there is provided a VH3 binding polypeptide wherein sequence viii) corresponds to the sequence from position 8 to position 54 in a sequence selected from the group consisting of SEQ ID NO:14 and SEQ ID NO:15. In one embodiment there is provided a VH3 binding polypeptide wherein vi) corresponds to the sequence from position 8 to position 54 in a sequence selected from the group consisting of SEQ ID NO:42-43 and 45-82; such as the group consisting of SEQ ID NO:46-48, 50-52 and 54-82; such as the group consisting of SEQ ID NO:47-48, 51 and 54-82; such as the group consisting of SEQ ID NO:47-48, 51, 54 and 56-82; such as the group consisting of SEQ ID NO:47, 51, 54 and 56-82; such as the group consisting of SEQ ID NO:54 and 56-82; such as the group consisting of SEQ ID NO:54 and 56-81; such as the group consisting of SEQ ID NO:54 and 56. In one embodiment there is provided a VH3 binding polypeptide wherein sequence vi) corresponds to the sequence from position 8 to position 54 in a sequence selected from the group consisting of SEQ ID NO:56-82; such as the group consisting of SEQ ID NO 56-81 or the group consisting of SEQ ID NO:56 and 82. In one embodiment there is provided a VH3 binding polypeptide wherein sequence vi) corresponds to the sequence from position 8 to position 54 in a sequence selected from the group consisting of SEQ ID NO:55 and SEQ ID NO:56. In one embodiment there is provided a VH3 binding polypeptide wherein sequence viii) corresponds to the sequence from position 8 to position 54 in SEQ ID NO:13, SEQ ID NO:14 or SEQ ID NO:15. In one embodiment there is provided a VH3 binding polypeptide wherein sequence vi) corresponds to the sequence from position 8 to position 54 in SEQ ID NO:54, SEQ ID NO:55 or SEQ ID NO:56.

Additionally, also encompassed by the present disclosure are VH3 binding polypeptides, which are listed in FIG. 5 as SEQ ID NO:83-87. Thus, in one embodiment there is provided a VH3 binding polypeptide

comprising a sequence selected from group consisting of a sequence corresponding to residues 8-54 in SEQ ID NO:83; a sequence corresponding to residues 8-54 in SEQ ID NO:84; a sequence corresponding to residues 8-54 in SEQ ID NO:85; a sequence corresponding to residues 11-57 in SEQ ID NO:86 and residues 1-47 in SEQ ID NO:87.

In some embodiments, the binding module Sequence C may form part of a polypeptide comprising an amino acid sequence further comprising Sequence 1 [S1] and/or Sequence 2 [S2]. In one embodiment, there is provided a VH3 binding polypeptide as defined herein, comprising a Sequence 1 [S1], [binding module sequence C] and Sequence 2 [S2] in the following order from the N-terminus to the C-terminus
[S1]-[binding module sequence C]-[S2],
   wherein [S1] or [S2] may be present or absent and [binding module sequence C] is as defined herein, and
   wherein, independently from each other,
[S1] is selected from the group consisting of:

(SEQ ID NO: 124)
ADNKFNK;
(SEQ ID NO: 125)
VDAKFDK;
(SEQ ID NO: 126)
VDNKFNK;
(SEQ ID NO: 127)
ADAQQNKFNK;
(SEQ ID NO: 128)
IAAKHDK;
(SEQ ID NO: 129)
IAAQHDK;
(SEQ ID NO: 130)
ADNNFNK;
(SEQ ID NO: 131)
IAAKFDE;
(SEQ ID NO: 132)
PAAKHDK;
(SEQ ID NO: 133)
ADNAFNT;
(SEQ ID NO: 134)
FNK; ADNRFNE;
(SEQ ID NO: 135)
IDSKFDE;
(SEQ ID NO: 136)
ADNRFNR;
(SEQ ID NO: 137)
ADNKHNK;
(SEQ ID NO: 138)
ADSKFDE;
(SEQ ID NO: 139)
IDAKHDE;
(SEQ ID NO: 140)
QQNKFNK;
(SEQ ID NO: 141)
ADNKFHK;
(SEQ ID NO: 142)
KFNK;
(SEQ ID NO: 143)
ADNNFNR;
(SEQ ID NO: 144)
AAAKHDK;
(SEQ ID NO: 145)
IDNKFNK;
(SEQ ID NO: 146)
IDAKFDE;
(SEQ ID NO: 147)
DNNFNK;
(SEQ ID NO: 148)
ADNKFNE;
(SEQ ID NO: 149)
AAAQHDK;
and
(SEQ ID NO: 150)
AAAKFDE;

and [S2] is selected from the group consisting of

(SEQ ID NO: 151)
QAPK;
(SEQ ID NO: 152)
QAPP;
QAP;
(SEQ ID NO: 153)
QAPR;
(SEQ ID NO: 154)
QAPE;
APK;
(SEQ ID NO: 155)
QAPG;
(SEQ ID NO: 156)
QAIK;
and
QA.

In one embodiment, S1 is selected from the group consisting of ADNKFNK; VDAKFDK; VDNKFNK; IAAKHDK; IAAQHDK; ADNNFNK; IAAKFDE; PAAKHDK; ADNAFNT: FNK; ADNRFNE; and IDSKFDE, such as the group consisting of ADNKFNK; VDAKFDK; VDNKFNK; IAAKHDK; IAAQHDK; ADNNFNK; IAAKFDE; and PAAKHDK, such as the group consisting of ADNKFNK; VDAKFDK; VDNKFNK; IAAKHDK; IAAQHDK; and ADNNFNK, such as the group consisting of ADNKFNK; VDAKFDK; VDNKFNK; IAAKHDK; and IAAQHDK, such as the group consisting of ADNKFNK; VDAKFDK; VDNKFNK; and IAAKHDK, such as the group consisting of ADNKFNK; VDAKFDK; and VDNKFNK. In one embodiment, S1 comprises or consists of VDNKFNK (SEQ ID NO:158). In one embodiment, S1 comprises or consists of VDAKFDK (SEQ ID NO:160). In one embodiment, S1 comprises or consists of ADNKFNK (SEQ ID NO:159).

In one embodiment, S2 is selected from the group consisting of QAPK; QAPP; QAP; QAPR; and QAPE, such as the group consisting of QAPK; QAPP; QAP; and QAPE, such as the group consisting of QAPK; QAPP; QAP; and QAPR, such as the group consisting of QAPK; QAPP and QAP; such as the group consisting of QAPK; and QAPP. In one embodiment, S2 comprises or consists of QAPK (SEQ ID NO: 51). In one embodiment, S2 comprises or consists of QAPP (SEQ ID NO:152). In one embodiment, S2 comprises or consists of QAP.

In some embodiments, the VH3 binding polypeptide may be selected from polypeptides of which Sequence A forms a part according to the following. The VH3 binding polypeptide may comprise an amino acid sequence selected from the group consisting of:

(SEQ ID NO: 157)
ADNKFNK-[Sequence A]-DPSQSANLLAEAKKLNDAQAPK;
(SEQ ID NO: 158)
VDNKFNK-[Sequence A]-DPSQSANLLAEAKKLNDAQAPK;
(SEQ ID NO: 159)
ADNKFNK-[Sequence A]-DPSVSKEILAEAKKLNDAQAPK;
(SEQ ID NO: 160)
VDAKFDK-[Sequence A]-DPSQSANLLAEAKKLNDAQAPK;
(SEQ ID NO: 161)
VDNKFNK-[Sequence A]-DPSVSKEILAEAKKLNDAQAPK;
(SEQ ID NO: 162)
VDAKFDK-[Sequence A]-DPSVSKAILAEAKKLNDAQAPK;
(SEQ ID NO: 163)
ADNNFNK-[Sequence A]-DPSQSANLLAEAKKLNESQAPK;
(SEQ ID NO: 164)
ADNKHNK-[Sequence A]-DPSQSANLLAEAKKLNDAQAPK;
(SEQ ID NO: 165)
ADNKFNK-[Sequence A]-DPSQSANLLAEAAPK;
(SEQ ID NO: 166)
IAAQHDK-[Sequence A]-DPSVSLEILAEAKKLNDAQAPK;
(SEQ ID NO: 167)
ADNKFHK-[Sequence A]-DPSQSANLLAEAKKLNDAQAPK;
(SEQ ID NO: 168)
FNK-[Sequence A]-DPSQSANLLAEAKKLNDAQAPK;
(SEQ ID NO: 169)
IAAQHDK-[Sequence A]-DPSVSLEILCEAKKLNDAQAPK;
(SEQ ID NO: 170)
IDAKFDE-[Sequence A]-DPSVSLALLAEAKKLNDAQAPP;
(SEQ ID NO: 171)
DNNFNK-[Sequence A]-DPSQSANLLAEAKKLNESQAPK;
(SEQ ID NO: 172)
VDAKFDK-[Sequence A]-DPSQSANLLAEAKKLNDAQAIK;
(SEQ ID NO: 173)
ADNRFNE-[Sequence A]-DPSVSKEILAEAKKLNDAQAPE;
and
(SEQ ID NO: 174)
IDAKFDE-[Sequence A]-DPSVSLSLLAEAKKLNDAQAPP;

wherein [Sequence A] is as defined herein.

In particular embodiments, the VH3 binding polypeptide comprises an amino acid sequence selected from the group consisting of:

(SEQ ID NO: 157)
ADNKFNK-[Sequence A]-DPSQSANLLAEAKKLNDAQAPK;
(SEQ ID NO: 158)
VDNKFNK-[Sequence A]-DPSQSANLLAEAKKLNDAQAPK;
(SEQ ID NO: 159)
ADNKFNK-[Sequence A]-DPSVSKEILAEAKKLNDAQAPK;
(SEQ ID NO: 160)
VDAKFDK-[Sequence A]-DPSQSANLLAEAKKLNDAQAPK;
(SEQ ID NO: 161)
VDNKFNK-[Sequence A]-DPSVSKEILAEAKKLNDAQAPK;
(SEQ ID NO: 162)
VDAKFDK-[Sequence A]-DPSVSKAILAEAKKLNDAQAPK;
(SEQ ID NO: 163)
ADNNFNK-[Sequence A]-DPSQSANLLAEAKKLNESQAPK;
(SEQ ID NO: 164)
ADNKHNK-[Sequence A]-DPSQSANLLAEAKKLNDAQAPK;
and
(SEQ ID NO: 165)
ADNKFNK-[Sequence A]-DPSQSANLLAEAAPK;

such as the group consisting of

(SEQ ID NO: 157)
ADNKFNK-[Sequence A]-DPSQSANLLAEAKKLNDAQAPK;
(SEQ ID NO: 158)
VDNKFNK-[Sequence A]-DPSQSANLLAEAKKLNDAQAPK;
(SEQ ID NO: 159)
ADNKFNK-[Sequence A]-DPSVSKEILAEAKKLNDAQAPK;
(SEQ ID NO: 160)
VDAKFDK-[Sequence A]-DPSQSANLLAEAKKLNDAQAPK;
(SEQ ID NO: 161)
VDNKFNK-[Sequence A]-DPSVSKEILAEAKKLNDAQAPK;
and
(SEQ ID NO: 162)
VDAKFDK-[Sequence A]-DPSVSKAILAEAKKLNDAQAPK;

such as the group consisting of

(SEQ ID NO: 157)
ADNKFNK-[Sequence A]-DPSQSANLLAEAKKLNDAQAPK;
(SEQ ID NO: 158)
VDNKFNK-[Sequence A]-DPSQSANLLAEAKKLNDAQAPK;
(SEQ ID NO: 159)
ADNKFNK-[Sequence A]-DPSVSKEILAEAKKLNDAQAPK;
(SEQ ID NO: 160)
VDAKFDK-[Sequence A]-DPSQSANLLAEAKKLNDAQAPK;
and
(SEQ ID NO: 162)
VDAKFDK-[Sequence A]-DPSVSKAILAEAKKLNDAQAPK;

such as the group consisting of:

(SEQ ID NO: 158)
VDNKFNK-[Sequence A]-DPSQSANLLAEAKKLNDAQAPK;
(SEQ ID NO: 160)
VDAKFDK-[Sequence A]-DPSQSANLLAEAKKLNDAQAPK;
(SEQ ID NO: 161)
VDNKFNK-[Sequence A]-DPSVSKEILAEAKKLNDAQAPK;
and
(SEQ ID NO: 162)
VDAKFDK-[Sequence A]-DPSVSKAILAEAKKLNDAQAPK;

such as the group consisting of

(SEQ ID NO: 158)
VDNKFNK-[Sequence A]-DPSQSANLLAEAKKLNDAQAPK;
and
(SEQ ID NO: 162)
VDAKFDK-[Sequence A]-DPSVSKAILAEAKKLNDAQAPK;

In one embodiment the VH3 binding polypeptide comprises or consists of VDNKFNK-[Sequence A]-DPSQSANLLAEAKKLNDAQAPK (SEQ ID NO:158). In one embodiment the VH3 binding polypeptide comprises or consists of VDAKFDK-[Sequence A]-DPSVSKAILAEAKKLNDAQAPK (SEQ ID NO:162).

In one embodiment, the VH3 binding polypeptide comprises an amino acid sequence selected from:

from the group consisting of:

xii)
(SEQ ID NO: 158)
VDNKFNK-[Sequence A]-DPSQSANLLAEAKKLNDAQAPK

wherein [Sequence A] is as defined herein;
xiii) an amino acid sequence which has at least 86% identity to the sequence defined in xii).

In one embodiment, the VH3 binding polypeptide comprises an amino acid sequence selected from the group consisting of:

xiv)
(SEQ ID NO: 162)
VDAKFDK-[Sequence A]-DPSVSKAILAEAKKLNDAQAPK

wherein [Sequence A] is as defined herein; and
xv) an amino acid sequence which has at least 86% identity to the sequence defined in xiv).

Again, polypeptides comprising minor changes as compared to the above amino acid sequences xii) or xiv) without largely affecting the tertiary structure and the function thereof are also within the scope of the present disclosure. Thus, in some embodiments, the VH3 binding polypeptides as defined above may for example have a sequence which is at least 87%, such as at least 89%, such as at least 91%, such as at least 93%, such as at least 94%, such as at least 96%, such as at least 98% identical to the sequence defined by xii) or xiv).

Sequence xiv) in such a polypeptide may be selected from the group consisting of SEQ ID NO:1-41, such as SEQ ID NO:1-40. Sequence xii) in such a polypeptide may be selected from the group consisting of SEQ ID NO:42-82, such as SEQ ID NO:42-81. Thus, in one embodiment sequence xii) or xiv) corresponds to a sequence selected from the group consisting of SEQ ID NO:1-82, such as the group consisting of SEQ ID NO:1-40 and 42-81, such as the group consisting of SEQ ID NO:1-15 and 42-56. In one embodiment, sequence xiv) corresponds to a sequence selected from the group consisting of SEQ ID NO:1-41, such as the group consisting of SEQ ID NO:1-40, such as the group consisting of SEQ ID NO:1-15. In one embodiment, said sequence xii) or xiv) corresponds to a sequence selected from the group consisting of SEQ ID NO:1, 2, 4-43 and 45-82; such as the group consisting of SEQ ID NO:5-7, 9-11, 13-41, 46-48, 50-52 and 54-82; such as the group consisting of SEQ ID NO:6-7, 10, 13-41, 47-48, 51 and 54-82; such as the group consisting of SEQ ID NO:6-7, 10, 13, 15-41, 47-48, 51, 54 and 56-82; such as the group consisting of SEQ ID NO:6, 10, 13, 15-41, 47, 51, 54 and 56-82; such as the group consisting of SEQ ID NO:13, 15-41, 54 and 56-82; such as the group consisting of SEQ ID NO:13, 15-40, 54 and 56-81; such as the group consisting of SEQ ID NO:13, 15, 54 and 56. In another embodiment, sequence xii) or xiv) corresponds to a sequence selected from the group consisting of SEQ ID NO:15-41 and 56-82; such as the group consisting of SEQ ID NO:15-40 and 56-81 or the group consisting of SEQ ID NO:15, 41, 56 and 82. In one embodiment, sequence xii) or xiv) corresponds to a sequence selected from the group consisting of SEQ ID NO:14, 15, 55 and 56. In one embodiment sequence xiv) corresponds to a sequence selected from the group consisting of SEQ ID NO:1, 2, 4-41; such as the group consisting of SEQ ID NO:5-7, 9-11, 13-41; such as the group consisting of SEQ ID NO:6-7, 10, 13-41; such as the group consisting of SEQ ID NO:6-7, 10, 13, 15-41; such as the group consisting of SEQ ID NO:6, 10, 13, 15-41; such as the group consisting of SEQ ID NO:13, 15-41; such as the group consisting of SEQ ID NO:13, 15-40; such as the group consisting of SEQ ID NO:13 and 15. In one embodiment, sequence xiv) corresponds to a sequence selected from the group consisting of SEQ ID NO:15-41; such as the group consisting of SEQ ID NO:15-40 or the group consisting of SEQ ID NO:15 and 41. In another embodiment, sequence xiv) corresponds to a sequence selected from the group consisting of SEQ ID NO:14 and 15. In yet another embodiment, sequence xii) corresponds to a sequence selected from the group consisting of SEQ ID NO:42-43 and 45-82; such as the group consisting of SEQ ID NO:46-48, 50-52 and 54-82; such as the group consisting of SEQ ID NO:47-48, 51 and 54-82; such as the group consisting of SEQ ID NO:47-48, 51, 54 and 56-82; such as the group consisting of SEQ ID NO:47, 51, 54 and 56-82; such as the group consisting of SEQ ID NO:54 and 56-82; such as the group consisting of SEQ ID NO:54 and 56-81; such as the group consisting of SEQ ID NO:54 and 56. In one embodiment, sequence xii) corresponds to a sequence selected from the group consisting of SEQ ID NO:56-82; such as the group consisting of SEQ ID NO 56-81 or the group consisting of SEQ ID NO:56 and 82. In one embodiment sequence xii) corresponds to a sequence selected from the group consisting of SEQ ID NO:55 and 56. In one embodiment, sequence xiv) corresponds to in SEQ ID NO:13, SEQ ID NO:14 or SEQ ID NO:15. And in another embodiment, sequence xii) corresponds to a sequence in SEQ ID NO:54, SEQ ID NO:55 or SEQ ID NO:56.

The VH3 binding polypeptide may also be selected from the sequences according to any one of SEQ ID NO:83-87. Thus, in one embodiment said VH3 binding polypeptide as disclosed herein comprises a sequence selected from group consisting of a sequence corresponding to residues 1-58 in SEQ ID NO:83; a sequence corresponding to residues 1-58 in SEQ ID NO:84; a sequence corresponding to residues 1-58 in SEQ ID NO:85; a sequence corresponding to residues 3-60 in SEQ ID NO:86 and a sequence corresponding to residues 1-51 in SEQ ID NO:87 or selected from group consisting of a sequence corresponding to residues 1-58 in SEQ ID NO:83; a sequence corresponding to residues 1-58 in SEQ ID NO:84; a sequence corresponding to residues 1-58 in SEQ ID NO:85; a sequence corresponding to residues 1-63 in SEQ ID NO:86 and a sequence corresponding to residues 1-51 in SEQ ID NO:87.

A further polypeptide domain with the same functionality may be attached to the VH3 binding polypeptide as defined herein, for example any VH3 binding polypeptide as defined in any one of i) to xv), thus achieving a VH3 binding polypeptide multimer. Such multimers may in particular be useful to increase the efficiency of binding the VH3 region, for example to ensure that more VH3 region containing polypeptides are bound to the VH3 binding polypeptide multimer compared to the monomer variants thereof (in other words the VH3 binding polypeptides). Thus, in a second aspect of the present disclosure, there is provided a VH3 binding polypeptide multimer. Said multimer is understood to comprise at least two VH3 binding polypeptides as disclosed herein as monomer units, the amino acid sequences of which may be the same or different. Thus, in one embodiment, there is provided a VH3 binding polypeptide multimer wherein each monomer of the multimer comprises a VH3 binding polypeptide which is independently selected from any VH3 binding polypeptide defined herein. In another embodiment, there is provided a VH3 binding polypeptide multimer wherein each monomer of the multimer comprises a Sequence A which is independently selected from any Sequence A defined herein.

Multimeric forms of the polypeptides may comprise a suitable number of domains, each having a VH3 binding activity, and each forming a monomer within the multimer. In one embodiment, said multimer is selected from the group consisting of dimer, trimer, tetramer, pentamer, hexamer, heptamer, octamer, nonamer and decamer, such as selected from the group consisting of dimer, tetramer, pentamer, hexamer, heptamer and octamer; such as the group consisting of dimer, tetramer and hexamer. In one particular embodiment, said multimer is a hexamer. In one particular embodiment, said multimer is a tetramer. In one embodiment, said multimer is a linear multimer, comprising sequentially arranged monomers. In one embodiment, said multimer is a linear polypeptide, comprising sequentially arranged monomers. In one embodiment, said each monomer is arranged such that the C-terminus thereof is adjacent to the N-terminus of the monomer immediately following it. In one embodiment, at least one monomer is arranged such that the C-terminus thereof is adjacent to the C-terminus of the monomer immediately following it.

The monomers within the multimer may have the same amino acid sequence, but alternatively, they may have different amino acid sequences. Thus the monomers may have any amino acid sequence of the VH3 binding polypeptides as defined herein, for example all monomers within the multimer may have different sequences or only a subset of the monomers in the multimer may have different amino acid sequences while other have the same sequence. In other words, the VH3 binding polypeptide of the invention may form homo- or heteromultimers, for example homo- or heterodimers. In one embodiment, there is provided a VH3 binding polypeptide multimer, wherein the multimer is a homomer. In another embodiment, there is provided a VH3 binding polypeptide multimer, wherein the multimer is a heteromer. Said heteromer may comprise at least two or at least three different Sequence A, wherein each Sequence A is as defined herein. In one embodiment, said multimer is a heteromer comprising at least two or at least three different VH3 binding polypeptides, each as defined herein. In one embodiment, said multimer is a homomer comprising four VH3 binding polypeptides as defined herein. In another embodiment said multimer is a homomer comprising six VH3 binding polypeptides as defined herein. In one particular embodiment, said multimer is a homomer or heteromer comprising at least three, such as at least four, such as at least five, such as at least six, such as at least seven, such as at least eight monomers selected from the group consisting of SEQ ID NO:1-82, such as the group consisting of SEQ ID NO:1-40 and 42-81, such as the group consisting of SEQ ID NO:1-15 and 42-56; such as the group consisting of SEQ ID NO:1-41, such as the group consisting of SEQ ID NO:1-40, such as the group consisting of SEQ ID NO:1-15. In one particular embodiment, said multimer is a homomer or heteromer comprising at least three, such as at least four, such as at least five, such as at least six, such as at least seven, such as at least eight monomers selected from the group consisting of SEQ ID NO:1, 2, 4-43 and 45-82; such as the group consisting of SEQ ID NO:5-7, 9-11, 13-41, 46-48, 50-52 and 54-82; such as the group consisting of SEQ ID NO:6-7, 10, 13-41, 47-48, 51 and 54-82; such as the group consisting of SEQ ID NO:6-7, 10, 13, 15-41, 47-48, 51, 54 and 56-82; such as the group consisting of SEQ ID NO:6, 10, 13, 15-41, 47, 51, 54 and 56-82; such as the group consisting of SEQ ID NO:13, 15-41, 54 and 56-82; such as the group consisting of SEQ ID NO:13, 15-40, 54 and 56-81; such as the group consisting of SEQ ID NO:13, 15, 54 and 56; or selected from the group consisting of SEQ ID NO:15-41 and 56-82; such as the group consisting of SEQ ID NO:15-40 and 56-81 or the group consisting of SEQ ID NO:15, 41, 56 and 82; or selected from the group consisting of SEQ ID NO:14, 15, 55 and 56. In one particular embodiment, said multimer is a homomer or heteromer comprising at least three, such as at least four, such as at least five, such as at least six, such as at least seven, such as at least eight monomers selected from the group consisting of SEQ ID NO:1, 2, 4-41; such as the group consisting of SEQ ID NO:5-7, 9-11, 13-41; such as the group consisting of SEQ ID NO:6-7, 10, 13-41; such as the group consisting of SEQ ID NO:6-7, 10, 13, 15-41; such as the group consisting of SEQ ID NO:6, 10, 13, 15-41; such as the group consisting of SEQ ID NO:13, 15-41; such as the group consisting of SEQ ID NO:13, 15-40; such as the group consisting of SEQ ID NO:13 and 15; or selected from the group consisting of SEQ ID NO:15-41; such as the group consisting of SEQ ID NO:15-40 or the group consisting of SEQ ID NO:15 and 41; or selected for the group consisting of SEQ ID NO:14 and 15. In one embodiment, said multimer is a homomer or heteromer comprising at least three, such as at least four, such as at least five, such as at least six, such as at least seven, such as at least eight monomers selected from the group consisting of SEQ ID NO:42-43 and 45-82; such as the group consisting of SEQ ID NO:46-48, 50-52 and 54-82; such as the group consisting of SEQ ID NO:47-48, 51 and 54-82; such as the group consisting of SEQ ID NO:47-48, 51, 54 and 56-82; such as the group consisting of SEQ ID NO:47, 51, 54 and 56-82; such as the group consisting of SEQ ID NO:54 and 56-82; such as the group consisting of SEQ ID NO:54 and 56-81; such as the group consisting of SEQ ID NO:54 and 56; or selected from the group consisting of SEQ ID NO:56-82; such as the group consisting of SEQ ID NO 56-81 or the group consisting of SEQ ID NO:56 and 82; or selected from the group consisting of SEQ ID NO:55 and 56. In one embodiment, said multimer is a homomer or heteromer comprising at least three, such as at least four, such as at least five, such as at least six, such as at least seven, such as at least eight monomers selected from the group consisting SEQ ID NO:13, SEQ ID NO:14 or SEQ ID NO:15; or selected from the group consisting of SEQ ID NO:54, SEQ ID NO:55 or SEQ ID NO:56. In one embodiment, said multimer comprises the sequence SEQ ID NO:13. In one embodiment, said multimer comprises the sequence SEQ ID NO:14. In one embodiment, said multimer comprises the sequence SEQ ID NO:15. In the appended Examples multimers of each of SEQ ID NO:13, SEQ ID NO:14 or SEQ ID NO:15 are described. Said multimers exhibit desirable properties in terms of VH3 affinity and stability when exposed to alkaline cleaning. Additionally, said multimers have low or no affinity for the Fc region of trastuzumab. Specific examples of such multimers are according to SEQ ID NO:175, 176 and 177. SEQ ID NO:175 comprises four SEQ ID NO:13, thus is a tetramer thereof. SEQ ID NO:175 comprises four SEQ ID NO:15, thus is a tetramer thereof. SEQ ID NO:177 comprises four SEQ ID NO:15, thus is a hexamer thereof.

It will be appreciated that the multimer may comprise any one of SEQ ID NO 83-87. In one embodiment, said multimer is a homomer or heteromer comprising at least three, such as at least four, such as at least five, such as at least six, such as at least seven, such as at least eight monomers selected from the group consisting of a sequence corresponding to residues 1-58 in SEQ ID NO:83; a sequence corresponding to residues 1-58 in SEQ ID NO:84; a sequence corresponding to residues 1-58 in SEQ ID NO:85; a sequence corresponding to residues 3-60 in SEQ ID NO:86 and residues 1-51 in SEQ ID NO:87 or selected from group consisting of a sequence corresponding to residues 1-58 in SEQ ID NO:83; a sequence corresponding to residues 1-58 in SEQ ID NO:84; a sequence corresponding to residues 1-58 in SEQ ID NO:85; a sequence corresponding to residues 1-63 in SEQ ID NO:86 and a sequence corresponding to residues 1-51 in SEQ ID NO:87.

It will be appreciated that monomers in the multimers discussed herein, which multimers comprise Sequence A or comprise VH3 binding polypeptides as disclosed herein, may be directly coupled to each other or spaced apart by linker sequences. The skilled person appreciates that the presence or absence of linker sequences may be different between different monomers moieties in a multimer, and if linkers are present, the sequence of each individual linker may be the same or different. Thus, in one embodiment, said VH3 binding polypeptide multimer comprises at least one linker. As the skilled person understands, the construction of a multimer, for example as a fusion protein, often involves use of linkers between the monomer moieties to be fused. The skilled person is aware of different kinds of linkers with different properties, such as flexible amino acid linkers, rigid amino acid linkers and cleavable amino acid linkers. Linkers may be used in order to for example increase stability or improve folding of fusion proteins, to increase expression or to improve activity, affinity and/or binding capacity, Thus, in one embodiment, the VH3 binding polypeptide multimer as defined herein further comprises at least one linker. In one embodiment, a linker is present between each monomer within the multimer. The linker may for example be selected from the group consisting of flexible amino acid linkers, rigid amino acid linkers and cleavable amino acid linkers. Alternatively, the linker may be a non-peptidic linker. Thus, the VH3 binding polypeptides disclosed herein may be linked to each directly by peptide bonds between the C-terminal and N-terminal ends of the polypeptides. Alternatively, two or more monomers, in other words monomer units or moieties, within the multimer can be linked by elements comprising oligomeric or polymeric species, such as elements comprising up to 15 or 30 amino acids, such as 1-5, 1-10 or 5-10 amino acids. In one embodiment, said linker comprises up to 15 amino acid residues. The nature of such a link should preferably not destabilize the spatial conformation of the protein units, that is of the VH3 binding polypeptide monomers within the multimer. This can e.g. be achieved by avoiding the presence of proline in the linkers. Furthermore, said linkers should preferably also be sufficiently stabile in alkaline environments not to impair the properties of the protein units. For this purpose, it is advantageous if the linkers do not contain asparagine. It can additionally be advantageous if the linker do not contain glutamine. The multimer may further at the N-terminal end comprise a plurality of amino acid residues originating from the cloning process or constituting a residue from a cleaved off signaling sequence. The number of additional amino acid residues may e.g. be 15 or less, such as 10 or less or 5 or less. The skilled person will understand that various modifications and/or additions can be made to a VH3 binding polypeptide or to a VH3 binding polypeptide multimer according to any aspect disclosed herein in order to tailor the polypeptide or multimer to a specific application without departing from the scope of the present disclosure.

For example, in one embodiment there is provided a VH3 binding polypeptide or a VH3 binding polypeptide multimer as described herein, which polypeptide or multimer has been extended by and/or comprises additional amino acids at the C terminus and/or N terminus. Such a polypeptide or multimer should be understood as a polypeptide or multimer having one or more additional amino acid residues at the very first and/or the very last position in the polypeptide chain, i.e. at the N- and/or C-terminus of the polypeptide or multimer. For example said additional amino acid residues may be at the N- and/or C-terminus any one of Sequence A according to i), ii) or iii), binding module Sequence C according to vi), vii), viii), ix), x), xi) or sequence xii), xiii), xiv) or xv). Thus, the VH3 binding polypeptides as defined herein may comprise any suitable number of additional amino acid residues, for example one, two, three, four, five, six, seven, eight, nine, ten or more additional amino acid residues. Said amino acid residues may individually or collectively improve production, purification, stabilization in vitro or coupling of the polypeptide to substrates of interest, for example to a solid support, such as a solid support described in connection to the aspect of a separation matrix.

Said additional amino residues may be coupled to the VH3 binding polypeptide or multimer by means of chemical conjugation (using known organic chemistry methods) or by any other means, such as expression of the VH3 binding polypeptide or multimer as a fusion protein or joined in any other fashion, either directly or via a linker, for example an amino acid linker as described above.

In some embodiments, the VH3 binding polypeptides and/or multimer, as disclosed above, further comprises at the C-terminal or N-terminal end one or more coupling elements, selected from the group consisting of a cysteine residue, a plurality of lysine residues and a plurality of histidine residues. The coupling element may e.g. be a single cysteine at the C-terminal end. The coupling element(s) may be directly linked to the C- or N-terminal end, or it/they may be linked via a linker comprising up to 15 amino acids, such as 1-5, 1-10 or 5-10 amino acids. This stretch should preferably also be sufficiently stabile in alkaline environments not to impair the properties of the protein. For this purpose, it is advantageous if the stretch does not contain asparagine. It can additionally be advantageous if the stretch does not contain glutamine. An advantage of having a C-terminal cysteine is that endpoint coupling of the protein can be achieved through reaction of the cysteine thiol with an electrophilic group on a support. This provides excellent mobility of the coupled protein which is important for the binding capacity.

The skilled person is aware there a numerous ways to produce a polypeptide. Thus in one embodiment, there is provided a VH3 binding polypeptide multimer as disclosed herein, wherein said VH3 binding polypeptide monomers are expressed as a fusion protein. In another embodiment, there is provided a VH3 binding polypeptide multimer as disclosed herein, wherein said VH3 binding polypeptide monomer units are covalently coupled together.

The terms “VH3 binding” and “binding affinity for VH3” as used in this disclosure refer to a property of a polypeptide which may be tested for example by ELISA or the use of surface plasmon resonance (SPR) technology.

VH3 binding affinity may be tested in an experiment in which VH3 containing polypeptide, or a fragment thereof, (also referred to sample below) is immobilized on a sensor chip of the surface plasmon resonance (SPR) instrument, and the sample containing the polypeptide to be tested is passed over the chip. Alternatively, the polypeptide to be tested is immobilized on a sensor chip of the instrument, and a sample containing VH3, or a fragment thereof, is passed over the chip. The skilled person may then interpret the results obtained by such experiments to establish at least a qualitative measure of the binding affinity of the polypeptide for VH3. If a quantitative measure is desired, for example to determine a K_{D} value for the interaction, surface plasmon resonance methods may also be used. Binding values may for example be defined in a Biacore™ (Cytiva™) or ProteOn™ XPR 36 (Bio-Rad™) instrument. The VH3 containing polypeptide, or a fragment thereof, is suitably immobilized on a sensor chip of the instrument, and samples of the polypeptide whose affinity is to be determined are prepared by serial dilution and injected in random order. A VH3 containing Fab may be used as target for the evaluation of VH3 binding affinity, K_{D} values may then be calculated from the results using for example the 1:1 Langmuir binding model of the BIAevaluation 4.1 software, or other suitable software, provided by the instrument manufacturer. The skilled person will appreciate that the VH3 containing Fab may be passed over the chip or may be immobilized on the sensor chip in the experimental setup as described above.

As explained above the VH3 binding polypeptide and VH3 binding polypeptide multimer of the present disclosure are able to bind to the VH3 region trastuzumab. Thus in one embodiment, there is provided a VH3 binding polypeptide or VH3 binding polypeptide multimer as disclosed herein, which is capable of binding to VH3 such as that the K_{D} value of the interaction is at most 1×10⁻⁶ M, such as at most 1×10⁻⁷ M, such as at most 1×10⁻⁸ M, such as at most 1×10⁻⁹ M, such as at most 1×10⁻¹⁰ M, such as at most 1×10⁻¹¹ M. For example, the K_{D} value may be calculated based on the interaction of a VH3 binding polypeptide or VH3 binding polypeptide multimer as disclosed herein and VH3 containing Fabs.

It will be appreciated that the VH3 binding polypeptides and/or the VH3 binding polypeptide multimers exhibit a similar K_{D} value of the interaction with VH3 as SEQ ID NO:88 and/or SEQ ID NO:89. In particular, the K_{D} value of the interaction with VH3 of the VH3 binding polypeptide or VH3 binding polypeptide multimer as disclosed herein may differ with one order of magnitude from the K_{D} value of the interaction of SEQ ID NO:88 and/or SEQ ID NO:89 with VH3. For example, if the K_{D} value of the interaction of SEQ ID NO:88 and/or SEQ ID NO:89 with a particular VH3 is 9×10⁻¹⁰ M in an assay, then the K_{D} value of the interaction with VH3 of the VH3 binding polypeptide or VH3 binding polypeptide multimer may be in the range of from 9×10⁻⁹ M to 9×10⁻¹¹ M (one order of magnitude), in that assay. The skilled person will appreciate that it may be useful to compare K_{D} values obtained using the same assay and while some variation may occur between different assays, intra-assay comparisons generally demonstrate the same trends independent of the assay employed. Assays may for example differ depending on if it is the VH3 contain polypeptide or the VH3 binding polypeptide which is immobilized on a chip. In one embodiment, said K_{D} value of the interaction with VH3 is measured in an assay wherein the VH3 binding polypeptide is immobilized on the chip. In a different embodiment, the K_{D} value of the interaction with VH3 is measured in an assay wherein the VH3 containing polypeptide is immobilized on the chip. In one embodiment, VH3 containing polypeptides is a VH3 containing Fab.

In another embodiment, there is provided a VH3 binding polypeptide or VH3 binding polypeptide multimer as disclosed herein, which is capable of binding to VH3 such as that the K_{D} value of the interaction is at least 25%, such as at least 30%, such as at least 40%, such as at least 50%, such as at least 60%, such as at least 70%, such as at least 80%, such as at least 90% of the K_{D} value of the interaction of SEQ ID NO:88 with VH3. In one embodiment, said VH3 binding polypeptide or VH3 binding polypeptide multimer is capable of binding to VH3 such as that the K_{D} value of the interaction is at least 25%, such as at least 30%, such as at least 40%, such as at least 50%, such as at least 60%, such as at least 70%, such as at least 80%, such as at least 90% of the K_{D} value of the interaction of SEQ ID NO:89 with VH3. In one embodiment, said VH3 binding polypeptide or VH3 binding polypeptide multimer is capable of binding to VH3 such as that the K_{D} value of the interaction is at least 25%, such as at least 30%, such as at least 40%, such as at least 50%, such as at least 60%, such as at least 70%, such as at least 80%, such as at least 90% of the K_{D} value of the interaction of SEQ ID NO:88 and SEQ ID NO:89 with VH3. The K_{D} value of the interaction with VH3 may be tested using a Fab comprising VH3 or using a full length antibody for example. For example, the measurements may be performed using the conditions as described in Example 1, 2 or 3. The skilled person appreciated that other VH3 containing polypeptides may be used when suitable.

Similarly, the terms “Fc binding” and “binding affinity for Fc” as used in this disclosure refer to a property of a polypeptide which may be tested for example by ELISA or the use of surface plasmon resonance (SPR) technology. Fc binding affinity may be tested in an experiment in which Fc, or a fragment thereof, is immobilized on a sensor chip of the surface plasmon resonance (SPR) instrument, and the sample containing the polypeptide to be tested is passed over the chip. Alternatively, the polypeptide to be tested (in this case the VH3 binding polypeptide) may be immobilized on a sensor chip of the instrument, and a sample containing Fc, or a fragment thereof, is passed over the chip. The skilled person appreciates that essentially an absence of binding is to be detected if the binding to Fc is abolished or significantly reduced. The skilled person may then interpret the results obtained by such experiments to establish at least a qualitative measure of the binding affinity of the polypeptide for Fc. If a quantitative measure is desired, for example to determine a K_{D} value for the interaction, surface plasmon resonance methods may also be used in an analogous manner to what is explained above of VH3 binding.

As explained in connection with the first aspect of the present disclosure, the VH3 binding polypeptides and VH3 binding polypeptide multimers as defined herein have may have significantly reduced or abolished binding affinity for Fc of trastuzumab. The skilled person will appreciate that the present VH3 binding polypeptides and VH3 binding polypeptide multimers do not significantly interact with Fc, and thus their capability to bind to Fc is low.

In one embodiment, there is provided a VH3 binding polypeptide or VH3 binding polypeptide multimer as disclosed herein, which is not capable of binding to Fc with a K_{D} value of the interaction of less than 1×10⁻⁴ M, such as less than 1×10⁻³ M.

In addition, the VH3 binding polypeptides and multimers thereof as disclosed herein displays improved alkali clean stability. In embodiments where said VH3 binding polypeptide or multimer is used for separation or isolation of e.g. antibodies/immunoglobulins having a VH3 region, high alkali stability will allow for use of highly alkaline conditions during cleaning, essential for long-term repeated use in a bioprocess separation setting.

In one embodiment, there is provided a VH3 binding polypeptide or VH3 binding polypeptide multimer as disclosed herein which retains at least 19%, such as at least 20%, such as at least 21%, such as at least 23%, such as at least 25%, such as at least 27%, such as at least 29%, such as at least 31%, such as at least 33%, such as at least 35%, such as at least 37%, such as at least 39%, such as at least 41%, such as at least 43%, such as at least 45%, such as at least 47%, such as at least 48%, such as at least 50% of its original VH3 binding capability after at least one cleaning step with 0.5 M NaOH. In one embodiment, said cleaning step comprises exposure to 0.5 M NaOH, such as incubation with 0.5 M NaOH, for a period of approximately 2-15 minutes, for example 5-15 minutes, such as for example approximately 5 minutes or approximately 10 minutes or approximately 15 minutes. For example the time of incubation with 0.5 M NaOH may be 5±0.5 minutes, 6±0.5 minutes, 7±0.5 minutes, or 8±0.5 minutes, 9±0.5 minutes, 10±0.5 minutes, 11±0.5 minutes, 13±0.5 minutes, 14±0.5 minutes, 14±0.5 minutes or 15±0.5 minutes. The time of incubation may also be longer, such as for example approximately 20 minutes or approximately 30 minutes or even 60 minutes or more. The skilled person is familiar with appropriate time frames for cleaning of affinity columns with NaOH. In one embodiment, said VH3 binding polypeptide or VH3 binding polypeptide multimer as disclosed herein retains at least 19%, such as at least 20%, such as at least 21%, such as at least 23%, such as at least 25%, such as at least 27%, such as at least 29%, such as at least 31%, such as at least 33%, such as at least 35%, such as at least 37%, such as at least 39%, such as at least 41%, such as at least 43%, such as at least 45%, such as at least 47%, such as at least 48%, such as at least 50% of its original VH3 binding capability after at least 10, 20, 40, 50, 75 or 100 cleaning steps with 0.5 M NaOH. For example each cleaning step may be 10 minutes, thus the exposure time (thus incubation time) of the VH3 binding polypeptide or VH3 binding polypeptide multimer as disclosed herein to NaOH will thus be 1000 minutes if the cleaning step is performed 100 times.

In one embodiment, there is provided a VH3 binding polypeptide or VH3 binding polypeptide multimer as disclosed herein which retains at least 19%, such as at least 20%, such as at least 21%, such as at least 23%, such as at least 25%, such as at least 27%, such as at least 29%, such as at least 31%, such as at least 33%, such as at least 35%, such as at least 37%, such as at least 39%, such as at least 41%, such as at least 43%, such as at least 45%, such as at least 47%, such as at least 48%, such as at least 50% of its original VH3 binding capability after 100 repeated binding cycles followed by cleaning with 0.5 M NaOH. It will be understood that each binding cycle is followed by a cleaning step. A cleaning step may be for example approximately 5-15 minutes incubation in contact with 0.5 M NaOH, such as for example approximately 5 minutes or 10 minutes or 15 minutes. For example the time of incubation in contact with 0.5 M NaOH may be 5±0.5 minutes, 6±0.5 minutes, 7±0.5 minutes, or 8±0.5 minutes, 9±0.5 minutes 10±0.5 minutes, 11±0.5 minutes, 13±0.5 minutes, 14±0.5 minutes, 14±0.5 minutes or 15±0.5 minutes.

In one embodiment, there is provided a VH3 binding polypeptide or a VH3 binding polypeptide multimer as disclosed herein, which exhibits an alkali stability of at least 35%, such as at least 40%, such as at least 40%, such as at least 45%, such as at least 50%, such as at least 55%, such as at least 60%, such as at least 65%, such as at least 70%, such as at least 75%, such as at least 80%, such as at least 85%, such as at least 90%, such as at least 95% of the alkali stability of SEQ ID NO:88 or of SEQ ID NO:89 after incubation in 0.5 M NaOH, such as after repeated incubation with 0.5 M NaOH. In one embodiment, said VH3 binding polypeptide or VH3 binding polypeptide multimer has an alkali stability of at least 60%, such as at least 70%, such as at least 80%, such as at least 85%, such as at least 89%, such as at least 90%, such as at least 95%, such as at least 98%, such as at least 99% of the alkali stability of SEQ ID NO:88 after incubation in 0.5 M NaOH. In one embodiment, said VH3 binding polypeptide or VH3 binding polypeptide multimer has an alkali stability of at least 60%, such as at least 70%, such as at least 80%, such as at least 85%, such as at least 89%, such as at least 90%, such as at least 95%, such as at least 98%, such as at least 99%, of the alkali stability of SEQ ID NO:89 after incubation in 0.5 M NaOH. In one embodiment, said VH3 binding polypeptide or VH3 binding polypeptide multimer has an alkali stability of at least 60%, such as at least 70%, such as at least 80%, such as at least 85%, such as at least 89%, such as at least 90%, such as at least 95%, such as at least 98%, such as at least 99% of the alkali stability of SEQ ID NO:88 and SEQ ID NO:89 after incubation in 0.5 M NaOH. The skilled person will appreciate the alkaline clean stability of the polypeptides disclosed herein may be tested and compared to the alkaline clean stability of the SEQ ID NO:88 or 89 for example as described in the appended Examples.

In one embodiment, there is provided a VH3 binding polypeptide or VH3 binding polypeptide multimer as disclosed herein which retains at least 19%, such as at least 20%, such as at least 21%, such as at least 23%, such as at least 25%, such as at least 27%, such as at least 29%, such as at least 31%, such as at least 33%, such as at least 35%, such as at least 37%, such as at least 39%, such as at least 41%, such as at least 43%, such as at least 45%, such as at least 47%, such as at least 48%, such as at least 50% of its original VH3 binding capability after incubation in 0.5 M NaOH. In one embodiment, said incubation is at least for 12 hours, such as at least for 24 hours, such as at least 36 hours, such as at least 48 hours, such as at least 72 hours, such as least 96 hours or more. Said incubation may be for example at 22+/−2° C. Hence, the degree of VH3 binding capability may be for example evaluated after an incubation of the VH3 binding polypeptide or multimer thereof for 24 hours in 0.5 M NaOH at 22° C.

In a third aspect of the present disclosure, there is provided a polynucleotide encoding a VH3 binding polypeptide or a VH3 binding polypeptide multimer as described herein; an expression vector comprising said polynucleotide; and a host cell comprising said expression vector.

Also encompassed by this disclosure is a method of producing the VH3 binding polypeptide or the VH3 binding polypeptide multimer as described herein, comprising culturing said host cell under conditions permissive of expression of said polypeptide or multimer from its expression vector, and isolating the polypeptide or multimer.

The VH3 binding polypeptide or multimer as disclosed herein may alternatively be produced by non-biological peptide synthesis using amino acids and/or amino acid derivatives having protected reactive side-chains, the non-biological peptide synthesis comprising
step-wise coupling of the amino acids and/or the amino acid derivatives to form a polypeptide according to the first aspect or a multimer according to the second aspect having protected reactive side-chains,
removal of the protecting groups from the reactive side-chains of the polypeptide or multimer, and
folding of the polypeptide or multimer in aqueous solution.

In another aspect, the present invention discloses a separation matrix, comprising a VH3 binding polypeptide according to any embodiment disclosed above coupled to a solid support. Such a matrix is useful for separation of immunoglobulins or other VH3-containing proteins from e.g. a sample. Since the VH3 binding polypeptide or multimer thereof as disclosed herein has binding affinity to VH3 and have low or no binding affinity to Fc, the separation matrix may allow for efficient separation of VH3 containing proteins from any Fc containing proteins or impurities. As described above, the VH3 binding polypeptide displays improved alkali stability which will allow for use of highly alkaline conditions during cleaning of the separation matrix, essential for long-term repeated use in a bioprocess separation setting. The alkali stability of the matrix can be assessed by measuring the immunoglobulin-binding capacity, or VH3 binding capacity, before and after incubation in alkaline solutions at a specified temperature, e.g. 22+/−2° C. The incubation can e.g. be performed in 0.5 M or 1.0 M NaOH for a number of 5, 10 or 15 min cycles, such as for example 50, 100, 200 or 300 cycles, corresponding to a total incubation time of 12.5, 25, 50 or 75 h.

The VH3 binding capacity of the matrix after 96-100 15 min incubation cycles or a total incubation time of 24 or 25 h in 0.5 M NaOH at 22+/−2° C. can be at least 80%, such as at least 85%, at least 90% or at least 95% of the VH3 capacity before the incubation. The capacity of the matrix after a total incubation time of 24 h in 1.0 M NaOH at 22+/−2° C. can be at least 70%, such as at least 80% or at least 90% of the VH3 capacity before the incubation. The VH3 binding capacity of the separation matrix may be measured as the dynamic binding capacity (DBC). Thus, the dynamic binding capacity of separation matrix after 96-100 15 min incubation cycles or a total incubation time of 24 or 25 h in 0.5 M NaOH at 22+/−2° C. may be at least 80%, such as at least 85%, at least 90% or at least 95% of its DBC before the incubation. The DBC of separation matrix after a total incubation time of 24 h in 1.0 M NaOH at 22+/−2° C. may be at least 70%, such as at least 80% or at least 90% of the DBC before the incubation.

In one embodiment, the separation matrix comprises a VH3 binding polypeptide multimer, wherein each monomer comprises a VH3 binding polypeptide as defined herein. The multimer, which is a homo-multimer or a hetero-multimer, may be a dimer, trimer, tetramer, pentamer, hexamer, heptamer, octamer, nonamer or decamer. For example, the separation matrix may comprise a hexamer of a VH3 binding polypeptide coupled to the solid support. For example, the separation matrix may comprise a tetramer of a VH3 binding polypeptide coupled to the solid support. Each monomer of the VH3 binding polypeptide multimer may comprise a Sequence A which is independently selected from any Sequence A defined herein. In particular, the separation matrix may comprise a sequence selected from the group consisting of SEQ ID NO:1-82, such as the group consisting of SEQ ID NO:1-40 and 42-81, such as the group consisting of SEQ ID NO:1-15 and 42-56; such as the group consisting of SEQ ID NO:1-41, such as the group consisting of SEQ ID NO:1-40, such as the group consisting of SEQ ID NO:1-15. In one embodiment, the separation matrix comprises a sequence selected from the group consisting of SEQ ID NO:1, 2, 4-43 and 45-82; such as the group consisting of SEQ ID NO:5-7, 9-11, 13-41, 46-48, 50-52 and 54-82; such as the group consisting of SEQ ID NO:6-7, 10, 13-41, 47-48, 51 and 54-82; such as the group consisting of SEQ ID NO:6-7, 10, 13, 15-41, 47-48, 51, 54 and 56-82; such as the group consisting of SEQ ID NO:6, 10, 13, 15-41, 47, 51, 54 and 56-82; such as the group consisting of SEQ ID NO:13, 15-41, 54 and 56-82; such as the group consisting of SEQ ID NO:13, 15-40, 54 and 56-81; such as the group consisting of SEQ ID NO:13, 15, 54 and 56; or selected from the group consisting of SEQ ID NO:15-41 and 56-82; such as the group consisting of SEQ ID NO:15-40 and 56-81 or the group consisting of SEQ ID NO:15, 41, 56 and 82; or selected from the group consisting of SEQ ID NO:14, 15, 55 and 56. In one embodiment, the separation matrix comprises a sequence selected from the group consisting of SEQ ID NO:1, 2, 4-41; such as the group consisting of SEQ ID NO:5-7, 9-11, 13-41; such as the group consisting of SEQ ID NO:6-7, 10, 13-41; such as the group consisting of SEQ ID NO:6-7, 10, 13, 15-41; such as the group consisting of SEQ ID NO:6, 10, 13, 15-41; such as the group consisting of SEQ ID NO:13, 15-41; such as the group consisting of SEQ ID NO:13, 15-40; such as the group consisting of SEQ ID NO:13 and 15; or selected from the group consisting of SEQ ID NO:15-41; such as the group consisting of SEQ ID NO:15-40 or the group consisting of SEQ ID NO:15 and 41; or selected for the group consisting of SEQ ID NO:14 and 15. In one embodiment, the separation matrix comprises a sequence selected from the group consisting of SEQ ID NO:42-43 and 45-82; such as the group consisting of SEQ ID NO:46-48, 50-52 and 54-82; such as the group consisting of SEQ ID NO:47-48, 51 and 54-82; such as the group consisting of SEQ ID NO:47-48, 51, 54 and 56-82; such as the group consisting of SEQ ID NO:47, 51, 54 and 56-82; such as the group consisting of SEQ ID NO:54 and 56-82; such as the group consisting of SEQ ID NO:54 and 56-81; such as the group consisting of SEQ ID NO:54 and 56; or selected from the group consisting of SEQ ID NO:56-82; such as the group consisting of SEQ ID NO 56-81 or the group consisting of SEQ ID NO:56 and 82; or selected from the group consisting of SEQ ID NO:55 and 56. In one embodiment, the separation matrix comprises a sequence selected from the group consisting of SEQ ID NO:13, SEQ ID NO:14 or SEQ ID NO:15; or selected from the group consisting of SEQ ID NO:54, SEQ ID NO:55 or SEQ ID NO:56. In one embodiment, said separation matrix comprises the sequence SEQ ID NO:13. In one embodiment, said separation matrix comprises the sequence SEQ ID NO:14. In one embodiment, said separation matrix comprises a VH3 binding polypeptide multimer, optionally a hexamer or tetramer, wherein each monomer of said multimer comprises a sequence corresponding to SEQ ID NO:15.

The separation matrix may comprise a sequence selected from the group consisting of SEQ ID NO:83-87. In one embodiment, said separation matrix comprises a VH3 binding polypeptide multimer, optionally a hexamer or tetramer, wherein each monomer of said multimer comprises a sequence corresponding any one of the above-mentioned sequences. In one embodiment, said separation matrix comprises a VH3 binding polypeptide multimer, optionally a hexamer or tetramer, wherein each monomer of said multimer comprises a sequence corresponding to SEQ ID NO:13. In one embodiment, said separation matrix comprises a VH3 binding polypeptide multimer, optionally a hexamer or tetramer, wherein each monomer of said multimer comprises a sequence corresponding to SEQ ID NO:14. In one embodiment, said separation matrix comprises a VH3 binding polypeptide multimer, optionally a hexamer or tetramer, wherein each monomer of said multimer comprises a sequence corresponding to SEQ ID NO:15. The separation matrix may comprise a sequence selected from the group consisting of SEQ ID NO:175-177, such as the group consisting of SEQ ID NO:176 and 177. In one embodiment, the separation matrix comprises SEQ ID NO:175. In one embodiment, the separation matrix comprises SEQ ID NO:176. In one embodiment, the separation matrix comprises SEQ ID NO:177. As the skilled person will understand, the expressed VH3 binding polypeptide, in monomeric or multimeric form, should be purified to an appropriate extent before being immobilized to a support. Such purification methods are well known in the field, and the immobilization of protein-based ligands to supports is easily carried out using standard methods. Suitable methods and supports will be discussed below in more detail and are disclosed e.g. in WO16079033 which is incorporated herein by reference.

The solid support of the matrix according to the invention can be of any suitable well-known kind. As a non-limiting example, a conventional affinity separation matrix is often of organic nature and based on polymers that expose a hydrophilic surface to the aqueous media used, i.e. expose hydroxy (—OH), carboxy (—COOH), carboxamido (—CONH2, possibly in N-substituted forms), amino (—NH2, possibly in substituted form), oligo- or polyethylenoxy groups on their external and, if present, also on internal surfaces. The solid support can suitably be porous. The porosity can be expressed as a Kav or Kd value (the fraction of the pore volume available to a probe molecule of a particular size) measured by inverse size exclusion chromatography, e.g. according to the methods described in Gel Filtration Principles and Methods, Pharmacia LKB Biotechnology 1991, pp 6-13. By definition, both Kd and Kav values always lie within the range 0-1. The Kav value can advantageously be 0.6-0.95, e.g. 0.7-0.90 or 0.6-0.8, as measured with dextran of Mw 110 kDa as a probe molecule. An advantage of this is that the support has a large fraction of pores able to accommodate both the polypeptides/multimers of the invention and immunoglobulins binding to the polypeptides/multimers and to provide mass transport of the immunoglobulins to and from the binding sites.

The polypeptides or multimers may be attached to the support via conventional coupling techniques utilizing e.g. thiol, amino and/or carboxy groups present in the ligand. Bisepoxides, epichlorohydrin, CNBr, N-hydroxysuccinimide (NHS) etc. are well-known coupling reagents. Between the support and the polypeptide/multimer, a molecule known as a spacer can be introduced, which improves the availability of the polypeptide/multimer and facilitates the chemical coupling of the polypeptide/multimer to the support. Depending on the nature of the polypeptide/multimer and the coupling conditions, the coupling may be a multipoint coupling (e.g. via a plurality of lysines) or a single point coupling (e.g. via a single cysteine).

Alternatively, the polypeptide/multimer may be attached to the support by non-covalent bonding, such as physical adsorption or biospecific adsorption.

In some embodiments the matrix comprises 5-25, such as 5-20 mg/ml, 5-15 mg/ml, 5-11 mg/ml or 6-11 mg/ml of the polypeptide or multimer coupled to the support. The amount of coupled polypeptide/multimer can be controlled by the concentration of polypeptide/multimer used in the coupling process, by the activation and coupling conditions used and/or by the pore structure of the support used. As a general rule the absolute binding capacity of the matrix increases with the amount of coupled polypeptide/multimer, at least up to a point where the pores become significantly constricted by the coupled polypeptide/multimer. The relative binding capacity per mg coupled polypeptide/multimer will decrease at high coupling levels, resulting in a cost-benefit optimum within the ranges specified above.

In certain embodiments the polypeptides or multimers are coupled to the support via thioether bonds. Methods for performing such coupling are well-known in this field and easily performed by the skilled person in this field using standard techniques and equipment. Thioether bonds are flexible and stabile and generally suited for use in affinity chromatography. In particular when the thioether bond is via a terminal or near-terminal cysteine residue on the polypeptide or multimer, the mobility of the coupled polypeptide/multimer is enhanced which provides improved binding capacity and binding kinetics. In some embodiments the polypeptide/multimer is coupled via a C-terminal cysteine provided on the protein as described above. This allows for efficient coupling of the cysteine thiol to electrophilic groups, e.g. epoxide groups, halohydrin groups etc. on a support, resulting in a thioether bridge coupling.

In certain embodiments the solid support comprises a polyhydroxy polymer, such as a polysaccharide. Examples of polysaccharides include e.g. dextran, starch, cellulose, pullulan, agar, agarose etc. Polysaccharides are inherently hydrophilic with low degrees of nonspecific interactions, they provide a high content of reactive (activatable) hydroxyl groups and they are generally stabile towards alkaline cleaning solutions used in bioprocessing. In some embodiments the support comprises agar or agarose. The supports used in the present invention can easily be prepared according to standard methods, such as inverse suspension gelation (S Hjerten: Biochim Biophys Acta 79(2), 393-398 (1964)). Alternatively, the base matrices are commercially available products, such as crosslinked agarose beads sold under the name of SEPHAROSE™ FF (Cytiva™). In an embodiment, which is especially advantageous for large-scale separations, the support has been adapted to increase its rigidity using the methods described in U.S. Pat. No. 6,602,990 or U.S. Pat. No. 7,396,467, which are hereby incorporated by reference in their entirety, and hence renders the matrix more suitable for high flow rates. In certain embodiments the support, such as a polysaccharide or agarose support, is crosslinked, such as with hydroxyalkyl ether crosslinks. Crosslinker reagents producing such crosslinks can be e.g. epihalohydrins like epichlorohydrin, diepoxides like butanediol diglycidyl ether, allylating reagents like allyl halides or allyl glycidyl ether. Crosslinking is beneficial for the rigidity of the support and improves the chemical stability. Hydroxyalkyl ether crosslinks are alkali stabile and do not cause significant nonspecific adsorption.

Alternatively, the solid support is based on synthetic polymers, such as polyvinyl alcohol, polyhydroxyalkyl acrylates, polyhydroxyalkyl methacrylates, polyacrylamides, polymethacrylamides etc. In case of hydrophobic polymers, such as matrices based on divinyl and monovinyl-substituted benzenes, the surface of the matrix is often hydrophilised to expose hydrophilic groups as defined above to a surrounding aqueous liquid. Such polymers are easily produced according to standard methods, see e.g. “Styrene based polymer supports developed by suspension polymerization” (R Arshady: Chimica e L'Industria 70(9), 70-75 (1988)). As an alternative, a commercially available product, such as SOURCE™ (Cytiva™) may be used. Alternatively, the solid support according to the invention comprises a support of inorganic nature, e.g. silica, zirconium oxide etc.

In one embodiment, the solid support has the form of a porous monolith. In an alternative embodiment, the support is in beaded or particle form that can be porous or non-porous. Solid supports in beaded or particle form can be used as a packed bed or in a suspended form. Suspended forms include those known as expanded beds and pure suspensions, in which the particles or beads are free to move. In case of monoliths, packed bed and expanded beds, the separation procedure commonly follows conventional chromatography with a concentration gradient. In case of pure suspension, batch-wise mode will be used.

In one embodiment, the separation matrix is a convection-based chromatography matrix. Such convection-based chromatography matrix may comprise a porous polymer membrane, a fibrous support or fibrous substrate. Examples of porous polymer membrane includes Mustang™ membranes from Pall and Sartobind™ membranes from Sartorius. Said fibrous support may be based on electrospun polymeric fibers or cellulose fibers, optionally non-woven fibers. The fibrous support may thus be a fibrous non-woven polymer matrix. The fibers comprised in said fibrous support have a cross-sectional diameter of 10-1000 nm, such as 200-800 nm, 200-400 nm or 300-400 nm. Such a fibrous support can be found in a HiTrap Fibro™ unit (Cytiva™). Alternative fibrous supports are disclosed in e.g. WO2019/137869 and WO2018/011600.

In yet another embodiment, the solid support is in another form such as a surface, a chip, capillaries, or a filter (e.g. a membrane or a depth filter matrix).

In another aspect, the present invention provides a method of isolating an immunoglobulin or fragment thereof, wherein a separation matrix as disclosed above is used. In some embodiments, the method comprises a) contacting a liquid sample comprising an immunoglobulin or a fragment thereof with a separation matrix as disclosed above. The method may furthermore comprise b) washing said separation matrix with a washing liquid, c) eluting the immunoglobulin from the separation matrix with an elution liquid, and optionally d) cleaning the separation matrix with a cleaning liquid. The cleaning liquid can alternatively be called a cleaning-in-place (CIP) liquid. The contact (incubation) time may be at least 10 min.

The method may also comprise steps of, before step a), providing an affinity separation matrix according to any of the embodiments described above and providing a solution comprising an immunoglobulin or a fragment thereof and at least one other substance as a liquid sample and of, after step c), recovering the eluate and optionally subjecting the eluate to further separation steps, e.g. by anion or cation exchange chromatography, multimodal chromatography and/or hydrophobic interaction chromatography. Suitable compositions of the liquid sample, the washing liquid and the elution liquid, as well as the general conditions for performing the separation are well known in the art of affinity chromatography and in particular in the art of Protein A chromatography. The liquid sample comprising a VH3-containing protein and at least one other substance may comprise host cell proteins (HCP), such as CHO cell, E. coli or yeast proteins. Contents of CHO cell and E. coli proteins can conveniently be determined by immunoassays directed towards these proteins, e.g. the CHO HCP or E. coli HCP ELISA kits from Cygnus Technologies. The host cell proteins or CHO cell/E. coli proteins may be desorbed during step b). The elution may be performed by using any suitable solution used for elution from Protein A media. This can e.g. be a solution or buffer with pH 5 or lower, such as pH 2.5-5 or 3-5. It can also in some cases be a solution or buffer with pH 11 or higher, such as pH 11-14 or pH 11-13. In some embodiments the elution buffer or the elution buffer gradient comprises at least one mono- di- or trifunctional carboxylic acid or salt of such a carboxylic acid. In certain embodiments the elution buffer or the elution buffer gradient comprises at least one anion species selected from the group consisting of acetate, citrate, glycine, succinate, phosphate, and formiate.

In some embodiments, the cleaning liquid is alkaline, such as with a pH of 13-14. Such solutions provide efficient cleaning of the matrix, in particular at the upper end of the interval.

In certain embodiments, the cleaning liquid comprises 0.1-2.0 M NaOH or KOH, such as 0.5-2.0 or 0.5-1.0 M NaOH or KOH. These are efficient cleaning solutions, and in particular so when the NaOH or KOH concentration is above 0.1 M or at least 0.5 M. The high stability of the polypeptides of the invention enables the use of such strongly alkaline solutions.

The method may also include a step of sanitizing the matrix with a sanitization liquid, which may e.g. comprise a peroxide, such as hydrogen peroxide and/or a peracid, such as peracetic acid or performic acid.

In some embodiments, steps a)-d) are repeated at least 10 times, such as at least 50 times, 50-200, 50-300 or 50-500 times. This is important for the process economy in that the matrix can be re-used many times. Steps a)-c) can also be repeated at least 10 times, such as at least 50 times, 50-200, 50-300 or 50-500 times, with step d) being performed after a plurality of instances of step c), such that step d) is performed at least 10 times, such as at least 50 times. Step d) can e.g. be performed every second to twentieth instance of step c).

The method of isolating an immunoglobulin or fragment as disclosed herein may be used for the isolation of fragments which comprise at least one VH3 domain. Non-limiting examples of such fragments include Fab fragments, Fab′ fragments, F(ab′)2, scFab fragments, Fv fragments, scFv fragments, diabodies, triabodies and minibodies. The skilled person is familiar with other fragments which the isolation of may be performed by the present method. Thus, in one embodiment of said method, the fragment is selected from Fab fragments, Fab′ fragments, F(ab′)2, scFab fragments, Fv fragments, scFv fragments, diabodies, triabodies and minibodies.

In particular, the present method may be suitable for the isolation of immunoglobulins or fragments thereof which immunoglobulin or fragment thereof is an asymmetric immunoglobulin or fragment thereof. As used herein, the term “asymmetric immunoglobulin or fragment thereof” refers to such immoglobulins or fragments wherein two different heavy chains or fragments of heavy chains are paired. Such immunoglobulins or fragments may be bispecific. One illustrative example of an asymmetric immunoglobulin is the asymmetric bispecific antibody Emicizumab (Chugai Pharmaceutical Co., Ltd.) which is used for the treatment of Hemophilia A. Emicizumab has two heavy chains, one with VH3 class and one with VH1 class. The present method allows for efficient isolation of correctly paired variants (VH3-VH1) by using the VH3 interaction of one of the heavy chains. In this way it is possible to separate between homodimer (undesired VH3-VH3 and VH1-VH1) and hetero dimer (desired VH3-VH1) variants already in the capture step and thus simplifying the downstream process. Without being bound by theory, the separation is envisioned to work in principle as follows: the homodimer (VH1-VH1) does not bind and therefore goes in flow-through (FT); the homodimer (VH3-VH3) binds with two interactions, thus creating avidity effect and therefore binds more stringent compared to the correctly paired hetero-dimer VH1-VH3. Thus, the correctly paired heterodimer is expected to elute at a milder eluation conditions, such as milder pH, than the VH3-VH3 dimer. Thus, by eluting in a gradient or two using a step elution with different pH's, a resolution between VH1-VH3 and VH3-VH3 is achieved. This is illustrated in the appended Example 6. In particular, the low or abolished ability of the VH3 binding polypeptides and multimers thereof to bind to the Fc region of trastuzumab as disclosed herein advantageously improves the purity of the isolated fractions as any confounding Fc-interaction is not present or is very low.

Thus, in one embodiment of said method, the immunoglobulin or fragment thereof is an asymmetric immunoglobulin or fragment thereof. In particular, said asymmetric immunoglobulin or fragment thereof comprises a VH3 region. In one embodiment, said asymmetric immunoglobulin or fragment thereof is an multispecific immunoglobulin or fragment thereof, such as at least bispecific or trispecific immunoglobulin or fragment thereof.

As the skilled person will appreciate the conditions the immunoglobulin and/or fragments thereof are subjected to during isolation may affect their target binding ability. Thus, it may be beneficial that the elution step c) is performed under mild conditions, such as mild pH conditions as exemplified in the appended Examples 4.

Thus, in an embodiment of said method, the separation matrix comprises a VH3 binding polypeptide according to SEQ ID NO:14 or a multimer comprising SEQ ID NO:14 and pH-level at elution is above 4, such as approximately in the range of 4.10-4.20, such as approximately 4.15. In another embodiment, said separation matrix comprises a VH3 binding polypeptide according to SEQ ID NO:15 or a multimer comprising SEQ ID NO:15 and pH-level at elution is below 4, such as approximately in the range of 3.7-3.80, such as approximately 3.75.

In one embodiment of the herein described method, wherein the method relates to the isolation of an asymmetric immunoglobulin or fragment thereof it is envisioned that the elution of the immunoglobulin or fragment thereof from the separation matrix is in a pH-gradient or is by a stepwise elution with at least two different pH-levels. It will be appreciated that said method may involve elution of both the symmetric immunoglobulin or fragment thereof and the assymetric immunoglobulin or fragment thereof or elution of one thereof. Thus, in one embodiment there is provided a method of isolating an asymmetric immunoglobulin or fragment thereof comprising the steps of:
a) providing a liquid sample comprising a mixture of at least one symmetric immunoglobulin or fragment thereof and at least one asymmetric immunoglobulin or fragment thereof;
b) contacting said liquid sample with a separation matrix to which said symmetric immunoglobulin or fragment thereof and said asymmetric immunoglobulin or fragment thereof bind with different avidity, wherein said separation matrix is defined according to claim 14;
c) eluting said immunoglobulins or fragments thereof from the separation matrix, whereby said symmetric immunoglobulin or fragment thereof and said asymmetric immunoglobulin or fragment thereof are at least partially eluted separately from each other, and optionally
d) cleaning the separation matrix with a cleaning liquid.

It is envisioned that said eluting of said immunoglobulins or fragments thereof from the separation matrix, whereby said symmetric immunoglobulin or fragment thereof and said asymmetric immunoglobulin or fragment thereof are at least partially eluted separately from each other may be performed based on pH, ionic strength or a combination of both properties. The skilled person is familiar with different eluation techniques. For example, said eluation may be performed using a gradient or performed by step wise changes of the elution ligiid.

In one embodiment, there is provided a method wherein step c) involves c) eluting said immunoglobulins or fragments thereof from the separation matrix in a pH-gradient or a stepwise elution with at least two different pH-levels, whereby said symmetric immunoglobulin or fragment thereof and said asymmetric immunoglobulin or fragment thereof are at least partially eluted at different pH-levels.

In one embodiment, said asymmetric immunoglobulin or fragment thereof comprises one VH3 domain. In one embodiment, said symmetric immunoglobulin or fragment thereof comprises at least two VH3 domains. In one embodiment, said symmetric immunoglobulin or fragment thereof comprises at least two VH3 domains or no VH3 domains. It is to be understood that said VH3 domains are VH3 domains of trastuzumab.

As explained above, said cleaning liquid may be NaOH or KOH, such as 0.5 NaOH, or other suitable cleaning liquid for cleaning separation matrices known in the art and as discussed above. It will be appreciated that the embodiments discussed above, such as relating to eluation buffer or gradient, cleaning liquid, optional steps of sanitizing the matrix, and the number of times step a)-c) or a)-d) are repeated are equally relevant for the

method of isolating an asymmetric immunoglobulin or fragment thereof as for the method of isolating an immunoglobulin or fragment thereof and are not repeated here merely for the sake of brevity.

It is to be appreciated that step c) may comprise eluation of one but not the other of said symmetric immunoglobulin or fragment thereof and asymmetric immunoglobulin or fragment thereof. Thus in one embodiment of the method as described herein, there is provide a method comprising a step c) comprising eluting at least one of said symmetric immunoglobulin or fragment thereof and asymmetric immunoglobulin or fragment thereof from the separation matrix at a pH-level suitable of elution of one but not the other of said symmetric immunoglobulin or fragment thereof and asymmetric immunoglobulin or fragment thereof, whereby said symmetric immunoglobulin or fragment thereof and said asymmetric immunoglobulin or fragment thereof are at least partially eluted at different pH-levels.

In one embodiment of the method of isolating an asymmetric immunoglobulin or fragment thereof, said separation matrix is as defined herein. As explained above, the method allows for efficient separation of immunoglobulins or fragments thereof which have paired in an undesired manner (such as homomeric pairing (also referred to as symmetric) from those which are paired in a desired manner (such as heteromeric pairing, also referred to asymmetric).

It is to be appreciated that properties of the resin used may influence the separation efficiency for said immunoglobulins or fragments thereof. For example, the resolution may be improved using a solid support characterized by smaller particles or beads, for example agarose beads. Thus, in one embodiment, the separation matrix comprises a solid support with particle or bead size of at most 75 μm, such as at most 60 μm, such as at most 50 μm or less. In one embodiment, said particle or bead size is the diameter of the particle or bead. In one embodiment, said beads are agarose beads.

In particular embodiments there if provided a method of isolating an asymmetric immunoglobulin or fragment thereof as disclosed herein, wherein no more than 25%, such as no more than 20%, such as no more than 15%, such as no more than 10%, of the of the asymmetric immunoglobulin or fragment thereof is present in the same eluate as the symmetric immunoglobulin or fragment thereof. In particular embodiments there if provided a method of isolating an asymmetric immunoglobulin or fragment thereof as disclosed herein, wherein no more than 25%, such as no more than 20%, such as no more than 15%, such as no more than 10%, such as no more than 5%, such as no more that 3% or less, of the eluate contains both symmetric and asymmetric immunoglobulin or fragment thereof. To clarify, in this context, eluate is to be interpreted as fractions that contain a significant amount of protein which protein was bound to the separation matrix after contacting the protein sample with a separation matrix as disclosed herein. Other fractions are considered to be flow through.

In one embodiment there is provided a method of isolating an asymmetric immunoglobulin or fragment thereof wherein said asymmetric immunoglobulin or fragment thereof is an multispecific immunoglobulin or fragment thereof, such as at least bispecific or trispecific immunoglobulin or fragment thereof.

The terms “VH3 binding polypeptide” and “VH3 binding protein” mean a polypeptide or protein respectively, capable of binding to the VH3 of an antibody and includes e.g.

Protein A, or any fragment or fusion protein thereof that has maintained said binding property.

The term “linker” herein means an element linking two polypeptide units, monomers or domains to each other in a multimer.

The term “spacer” herein means an element connecting a polypeptide or a polypeptide multimer to a support.

The term “dynamic binding capacity (DBC)” of a chromatography column in the context of protein purification describes the maximum amount of target protein that one can load onto ones column without causing unnecessary loss, measured under realistic experimental conditions (default flow rate, real protein sample),

While the invention has been described with reference to various exemplary aspects and embodiments, it will be understood by those skilled in the art that various changes may be made and equivalents may be substituted for elements thereof without departing from the scope of the invention. In addition, many modifications may be made to adapt a particular situation or molecule to the teachings of the invention without departing from the essential scope thereof. Therefore, it is intended that the invention not be limited to any particular embodiment contemplated, but that the invention will include all embodiments falling within the scope of the appended claims.

### BRIEF DESCRIPTION OF THE FIGURES

FIG. 1A shows Biacore analysis of inventive VH3 binding polypeptides (SEQ ID NO:1 to 4) using 5000 nM trastuzumab Fc fragment as analyte in comparison to an alkali clean stabile positive control that binds both Fc and Fab fragments of trastuzumab (SEQ ID NO:88) and a non-Fc-binding control (SEQ ID NO:180). For the purpose of clear representation, results of the Biacore analysis are shown in two graphs in FIG. 1A, one including and one excluding data corresponding to SEQ ID NO:88.

FIG. 1B shows Biacore analysis of inventive VH3 binding polypeptides (SEQ ID NO:1 to 4) using 5000 nM trastuzumab Fab fragment as analyte in comparison to a non-Fc-binding control (SEQ ID NO:180).

FIG. 1C is an example plot of alkali clean stability assessed using Biacore™ for tested alkali clean stabile inventive VH3 binding polypeptides (SEQ ID NO:1 to 4) in comparison to an alkali clean stabile positive control that binds both Fc and Fab fragments of trastuzumab (SEQ ID NO:88) and a non-Fc-binding control that is not alkali clean stabile (Negative CTRL).

FIG. 2A shows Biacore analysis of inventive VH3 binding polypeptides (SEQ ID NO:5 to 12) using 5000 nM trastuzumab Fc fragment as analyte in comparison to an alkali clean stabile positive control that binds both Fc and Fab fragments of trastuzumab (SEQ ID NO:88). For the purpose of clear representation, results of the Biacore analysis are shown in two graphs in FIG. 2A, one including and one excluding data corresponding to SEQ ID NO:88.

FIG. 2B shows Biacore analysis of inventive VH3 binding polypeptides (SEQ ID NO:5 to 12) using 625 nM trastuzumab as analyte in comparison to an alkali clean stabile positive control that binds both Fc and Fab fragments of trastuzumab (SEQ ID NO:88). For the purpose of clear representation, results of the Biacore analysis are shown in two graphs in FIG. 2B, one including and one excluding data corresponding to SEQ ID NO:88.

FIG. 2C is an example plot of assessed using Biacore™ for tested alkali clean stabile inventive VH3 binding polypeptides (SEQ ID NO:5 to 12) in comparison to an alkali clean stabile positive control that binds both Fc and Fab fragments of trastuzumab (SEQ ID NO:88) and a non-Fc-binding control that is not alkali clean stabile (Negative CTRL).

FIG. 3A shows Biacore analysis of inventive VH3 binding polypeptides (SEQ ID NO:13 to 15) using 5000 nM trastuzumab Fc fragment as analyte in comparison to an alkali clean stabile positive control that binds both Fc and Fab fragments of trastuzumab (SEQ ID NO:88). For the purpose of clear representation, results of the Biacore analysis are shown in two graphs in FIG. 3A, one including and one excluding data corresponding to SEQ ID NO:88.

FIG. 3B shows Biacore analysis of inventive VH3 binding polypeptides (SEQ ID NO:10 and SEQ ID NO:13 to 15) using 2500 nM trastuzumab Fab fragment as analyte in comparison to an alkali clean stabile positive control that binds both Fc and Fab fragments of trastuzumab (SEQ ID NO:88) and a non-Fc-binding control that is not alkali clean stabile (Negative CTRL).

FIG. 3C shows Biacore analysis of inventive VH3 binding polypeptides (SEQ ID NO:10 and SEQ ID NO:13 to 15) using 625 nM trastuzumab as analyte in comparison to an alkali clean stabile positive control that binds both Fc and Fab fragments of trastuzumab (SEQ ID NO:88) and a non-Fc-binding control that is not alkali clean stabile (Negative CTRL).

FIG. 3D is an example plot of alkali clean stability assessed using Biacore™ for tested alkali clean stabile inventive VH3 binding polypeptides (SEQ ID NO:10 and SEQ ID NO:13 to 15) in comparison to an alkali clean stabile positive control that binds both Fc and Fab fragments of trastuzumab (SEQ ID NO:88) and a non-Fc-binding control that is not alkali clean stabile (Negative CTRL).

FIG. 4A shows elution profile of emicizumab feed comprising VH1/VH3 bispecific antibodies and also VH3/VH3 mismatched homodimers using a pH gradient and a reference resin (SEQ ID NO:181).

FIG. 4B shows elution profile of emicizumab feed comprising VH1/VH3 bispecific antibodies and also VH3/VH3 mismatched homodimers using a pH gradient and a resin according to the present invention wherein SEQ ID NO:177 was coupled to agarose base matrix of 60 μm.

FIG. 4C shows elution profile of emicizumab feed comprising VH1/VH3 bispecific antibodies and also VH3/VH3 mismatched homodimers using a pH gradient and a resin according to the present invention wherein SEQ ID NO:177 was coupled to agarose base matrix of 50 μm.

FIG. 5 is a is a listing of the amino acid sequences of examples of VH3 binding polypeptides of the present disclosure (SEQ ID NO:1-82) and derivates of domain A, B, C, D and E of Staphylococcal protein A (SEQ ID NO:83-87). In the VH3 binding polypeptides of the present disclosure, the deduced Sequence A extend from residue 8 to residue 36 in each of SEQ ID NO:1-82. The amino acid sequences Sequence B extend from residue 37-54 and binding module Sequence C predicted to constitute the complete three-helix bundle within each of these VH3 binding polypeptides extend from residue 8 to residue 54 of the 58-mers.

### EXAMPLES

### Summary of Examples 1-10

The following examples disclose the surprising identification of novel VH3 binding SpA-derived polypeptides which exhibit no, or significantly reduced affinity for the Fc region of immunoglobulins and are alkali clean stabile.

A class of alkali clean stabile VH3 binding polypeptides was identified that show abolished trastuzumab Fc binding (SEQ ID NO:1-4). VH3 binding polypeptides (Fr1-4; SEQ ID NO:1-4) were evaluated in the following aspects: trastuzumab Fc binding, trastuzumab Fab binding and alkali clean stability (Example 1).

Additional alkali clean stabile VH3 binding polypeptides were identified that show no binding to the Fc fragment of trastuzumab (Fr5-12; SEQ ID NO:5-12). VH3 binding polypeptides (Fr5-12, SEQ ID NO:5 to 12) were evaluated in the following aspects: trastuzumab Fc binding, trastuzumab binding and alkali clean stability (Example 2).

Based on a VH3 binding polypeptide with improved alkali clean stability (SEQ ID NO:10) an additional set of alkali clean stabile VH3 binding polypeptides was identified that exhibit no trastuzumab Fc binding and show enhanced binding affinity for trastuzumab and the Fab fragment of trastuzumab (Fr31-33; SEQ ID NO:13-15). The VH3 binding polypeptides SEQ ID NO:13-15 were evaluated in the following aspects: trastuzumab Fc binding, trastuzumab Fab binding, trastuzumab binding and alkali clean stability (Example 3).

A subset of alkali clean stabile VH3 binding polypeptides with abolished Fc binding (Fr32 and Fr33; SEQ ID NO:14 and 15) were chosen for further studies on column. The further studies included multimerization of the polypeptides to tetramers and hexamers and expression of said multimers in E. coli. On these multimeric VH3 binding polypeptides (Fr32-4, Fr33-4 and Fr33-6; SEQ ID NO:175-177), a primary characterization was performed, wherein multimers of alkali clean stabile VH3 binding polypeptides were evaluated for dynamic binding capacity (DBC), pH elution and alkali clean stability (Example 4).

A VH3 binding polypeptide multimer variant (Fr33-6; SEQ ID NO:177) was chosen for further studies to evaluate binding specificity to different VH1 and VH3 class target molecules (Examples 5 and 6) and to evaluate binding specificity to VH1/VH3 asymmetric bispecific antibodies (Examples 6 and 7).

The effect of mutations in positions 9, 10, 11, 13, 14, 17, 18, 28 and 29 is evaluated in different scaffolds (SEQ ID NO:89-94) in the following aspects: trastuzumab Fc binding, trastuzumab Fab binding, trastuzumab binding and alkali clean stability (Examples 8 and 9).

Based on the sequence of Fr33 (SEQ ID NO:15 and 56) an additional novel class of VH3 binding polypeptides is made (SEQ ID NO:16-41 and SEQ ID NO:57-82) with amino acid variations in positions 13, 14 and 26. VH3 binding polypeptides (SEQ ID NO:16-41 and SEQ ID NO:57-82) are evaluated in the following aspects: trastuzumab Fc binding, trastuzumab Fab binding, trastuzumab binding and alkali clean stability (Example 10).

### Example 1

### Biacore Evaluation of VH3 Binding Polypeptides

### Summary

The impact of mutations was investigated to identify amino acid positions that enabled reduced binding to the Fc region while retaining alkali clean stability. A class of VH3 binding polypeptides (Fr1-4, SEQ ID NO:1 to 4) derived from SEQ ID NO:88 was identified as set out in Table 1. The polypeptides were evaluated in the following aspects: trastuzumab Fc binding, trastuzumab Fab binding and alkali clean stability.

TABLE 1
Summary of mutations (SEQ ID NO: 1 to 4)

| VH3 binding | SEQ | Position | | | | | | |
|---|---|---|---|---|---|---|---|---|
| polypeptide | ID NO: | 9 | 10 | 11 | 13 | 14 | 17 | 18 |
| Fr1 | 1 | Q | Q | T | L | A | | |
| Fr2 | 2 | Q | Y | T | L | A | | |
| Fr3 | 3 | Y | Q | T | L | A | | |
| Fr4 | 4 | Q | Q | T | L | A | A | R |

The polypeptide variants (SEQ ID NO:1-4) were evaluated together with an alkali clean stabile positive control that binds both Fc and Fab fragments of trastuzumab (SEQ ID NO:88), a non-Fc-binding control (SEQ ID NO:180) and a non-Fc-binding control that is not alkali clean stabile (Negative CTRL).

The following aspects were evaluated:
(1) Affinity assessment of Fc interaction (tested through high concentration injection of Fc fragment generated from trastuzumab).
(2) Affinity assessment of Fab interaction (tested through high concentration injection of Fab fragment generated from trastuzumab).
(3) Alkali clean stability (tested by reduction of trastuzumab binding after increasing number of treatments with 0.5 M NaOH).

The inventors surprisingly found a class of novel VH3 binding SpA-derived polypeptides (SEQ ID NO:1-4) with a diversity of amino acids at positions 9, 10, 11, 13, 14 and optionally at positions 17 and 18 that exhibit no, or significantly reduced affinity for the Fc region of trastuzumab without the loss of alkali clean stability.

### Materials and Methods

Generation of his-Purified Candidate Polypeptides

Materials and equipment used were as follows: glycerol stocks for SEQ ID NO:1 to 4; LB culture medium, 2YT culture medium, Carbenicillin, IPTG 1 M, 125 ml & 250 ml baffled glass shake flasks, Infors HT Shaking incubator; His-GraviTrap™ and kit (Cytiva™), PD10 GraviTraps (Cytiva™)

5 μl glycerol stock of each variant was inoculated in 4 ml LB supplemented with 200 μg/ml carbenicillin in 125 ml flasks and incubated at 37° C. overnight at 200 rpm.

Protein expression culture medium (2YT medium supplemented with 100 μg/ml carbenicillin) was prepared and added to filled baffled shake flasks (50 ml per flask, 20 flasks in total). Each flask was inoculated with approximately 500 μl from the previous overnight culture to obtain a starting OD600 of 0.05. The flasks were incubated in Infors HT shaking incubator for approximately 2.5 hours at 37° C. and 130 rpm shaking until OD600 reached 0.7. Thereafter 25 μl IPTG (1 M) was added and the flasks were incubated in Infors HT shaking incubator overnight at 27° C. and 130 rpm shaking, whereafter the cultures were pelleted (8000 g, 5 min) and re-suspended in 10 ml PBS+20 mM imidazole in Falcon tubes. To generate crude variant lysates the Falcon tubes were incubated in 80-85° C. water bath for 10 minutes followed by pelleting of cell debris by centrifugation at 10000 g for 10 min and filtration of supernatant with a 0.2 μm filter.

The samples were purified using His GraviTraps and kit according to kit instructions (PBS+20 mM imidazole was used as binding/wash buffer). Buffer exchange into PBS was performed using PD10 Gravitraps and kit instructions.

Concentration of buffer exchanged samples was measured using NanoDrop (Thermo Scientific) according to manufacturer's instructions. Following purification, samples were kept in refrigerator until all measurements performed and were then transferred to eppendorf tubes and kept in refrigerator.

### Biacore Analysis of Binding Affinity for Fc and Fab Fragments of Trastuzumab

To assess binding to Fc (SEQ ID NO:184) and Fab (SEQ ID NO:182 and 183), VH3 binding polypeptides (SEQ ID NO:1 to 4) were immobilized on a Biacore CM5 chip. Trastuzumab Fc (5000 nM) and trastuzumab Fab (5000 nM) fragments were used as analyte.

pH-Scouting for Immobilization Conditions:

Materials and equipment used were as follows: VH3 binding polypeptide variants (SEQ ID NO:1 to 4); Biacore™ CM5 chips (Cytiva™); Biacore™ Acetate-buffers (Cytiva™) with different pH; Biacore™ 8K+ instrument (Cytiva™)

The pH-scouting was performed on Biacore™ 8K+ using the standard method defined in the software at different pH values (pH 4.5, 5, 5.5). A pH value corresponding to one unit below the pI of the target ligand was found to give the best surface attraction.

### Biacore Binding Analysis:

Materials and equipment used were as follows: CM5 sensor chips (Cytiva™) Biacore™ NHS coupling kit (Cytiva™), VH3 binding polypeptides (SEQ ID NO:1 to 4); Biacore™ 8K+(Cytiva™); Fab (prepared from trastuzumab in-house), Fc from trastuzumab.

Immobilization was performed using a standard method in Biacore software with coupling of VH3 binding polypeptide variants in Flow Cell 2 (FC2) and activation/inactivation in Flow cell 1 (FC1). VH3 binding polypeptide variants were diluted in an appropriate pH (based on pH-scouting) in acetate buffer at a concentration ranging from 10-30 μg/ml. The immobilization levels did vary somewhat between different polypeptide variants (approx. 600-1300 Ru).

In each run the polypeptide was immobilized in FC2. Multiple chips were used until all candidates were tested.

Biacore™ method for binding analysis:
Running buffer: PBS-P+
Flow rate: 10 μl/min
Sample injection: 600 s/10 min over both Flow Cells (FC1 and FC2)
Dissociation time: 600 s/10 min
Regeneration: 10 mM Glycin-HCl pH 1.5, 30 μl/min, 2×30 s

Injections of analyte (Fab or Fc fragment of trastuzumab) for each channel (cycles) were as follows: buffer, 156 nM, 313 nM, 625 nM, 1250 nM, 2500 nM, 5000 nM.

All sensorgrams were generated as reference subtracted and the responses as the difference between baseline before injection and signal just before end of injection.

### Biacore Analysis of Alkali Clean Stability

Assessment of alkali clean stability was done by immobilizing the VH3 binding polypeptides (SEQ ID NO:1 to 4) on a CM5 Biacore chip followed by 100 repeating cycles of binding to trastuzumab (1 μM) followed by injection of NaOH (0.5 M).

Materials and equipment used were as follows: CM5 sensor chips (Cytiva™) Biacore™ NHS coupling kit (Cytiva™), VH3 binding polypeptides (SEQ ID NO:1 to 4); Biacore™ 8K+(Cytiva™); trastuzumab; 0.5 M NaOH.

Immobilization was performed using a standard method in Biacore software with coupling of VH3 binding polypeptide variants in FC2 and activation/inactivation in FC1. VH3 binding polypeptide variants were diluted in an appropriate pH (based on pH scouting) in acetate buffer at a concentration ranging from 10-30 μg/ml. The immobilization levels did vary somewhat between different polypeptide variants (approx. 600-1300 Ru).

Biacore method for alkaline stability (per cycle):
Running buffer: PBS-P+
Flow rate: 10 μl/min
Sample injection 1 (trastuzumab 1 μM): 300 s over both Flow Cells
Dissociation time 1: 60 s
Sample injection 2 (0.5 M NaOH): 600 s over both flow cells.
Regeneration: 10 mM Glycin-HCl pH 1.5, 30 μl/min, 2×30 s

This cycle was repeated more than 100 times to follow stability of trastuzumab response values.

All sensorgrams were generated as reference subtracted and the responses as the difference between baseline before injection and signal just before end of injection.

### Results

### Biacore Analysis of Binding Affinity for Fc and Fab Fragments of Trastuzumab

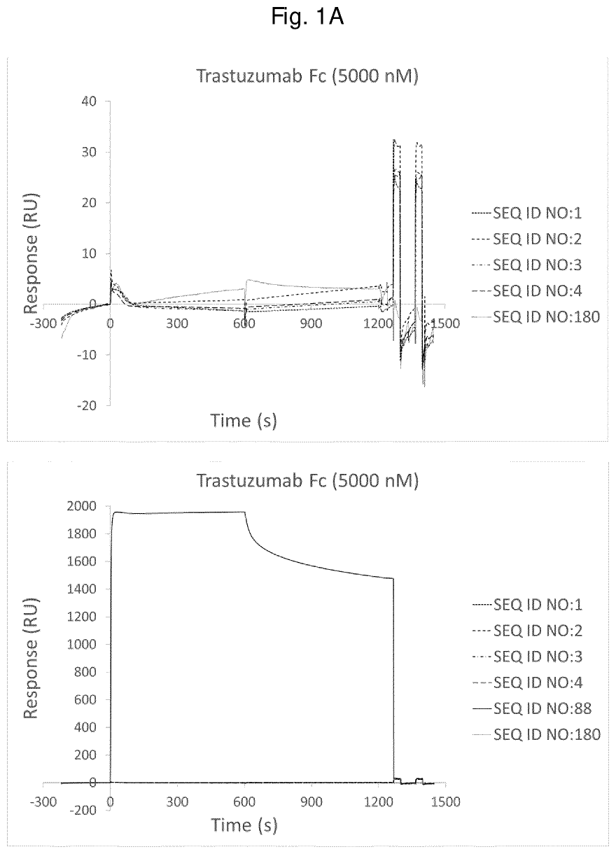

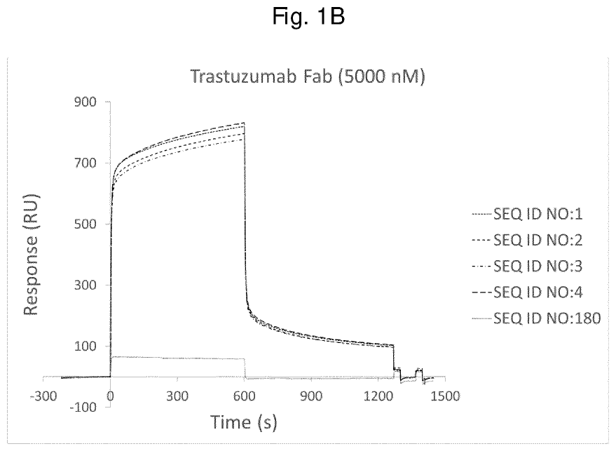

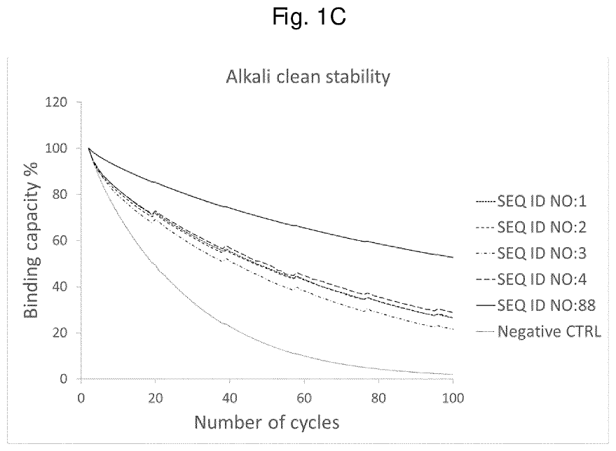

The response levels of the trastuzumab Fc and Fab interactions of the VH3 binding polypeptide variants are depicted in FIG. 1A and FIG. 1B. The data show that the VH3 binding polypeptide variants SEQ ID NO:1-4 exhibit a significantly reduced or abolished binding affinity for the Fc fragment of trastuzumab while retaining binding to the Fab fragment of trastuzumab. The positive reference, SEQ ID NO:88 shows binding to the Fc fragment of trastuzumab with 2000 RU at injection. The non-Fc-binding control (SEQ ID NO:180) shows no interaction with Fc (0 RU at injection), and weak binding to Fab with ˜100 RU at injection.

### Biacore Analysis of Alkali Clean Stability

Alkali clean stability was visualized by plotting normalized trastuzumab response values (in % of response in cycle 2) from the second cycle as set out above to the last cycle run. The alkali clean stabile positive control that binds both Fc and Fab fragments (SEQ ID NO:88) and a non-Fc-binding control that is not alkali clean stabile (Negative CTRL) were present as controls in each separate stability run. Trastuzumab response values after 100 cleaning cycles are summarized in Table 2. FIG. 1C shows plots of alkali clean stability for tested VH3 binding polypeptide variants (SEQ ID NO:1 to 4) in comparison to SEQ ID NO:88 and the non-alkali-clean-stabile, non-Fc-binding control (Negative CTRL). In each cycle, 0.5 M NaOH was injected with a contact time of 10 min at 10 μl/min.

TABLE 2
Trastuzumab response values (in % of
response in cycle 2) after 100 cycles

| | | Binding % after | |
|---|---|---|---|
| Polypeptide | SEQ ID NO | 100 cycles | RU cycle 2 |
| | 88 | 52.6 | 3597 |
| Fr4 | 4 | 28.7 | 2318 |
| Fr2 | 2 | 26.5 | 2432 |
| Fr1 | 1 | 26.4 | 2254 |
| Fr3 | 3 | 21.5 | 2147 |
| Negative CTRL | | 2 | 3322 |

### Conclusions

Novel VH3 binding polypeptides SEQ ID NO:1 to 4 were identified that show no binding to the Fc region of trastuzumab and show binding to the trastuzumab Fab fragment.

SEQ ID NO:1 to 4 are alkali clean stabile and retain at least 21.5% of their trastuzumab response (binding %) in cycle 2, after 100 cleaning cycles of 0.5M NaOH with a contact time of 10 min at 10 μl/min.

It is concluded that at least said combinations of amino acids at positions 9, 10, 11, 13, 14 and optionally 17 and 18 together, as set out in Table 1, lead to the advantageous properties of VH3 binding polypeptides according to the present invention: alkali clean stability, at least retain binding to the Fab fragment of trastuzumab and exhibit reduced or abolished binding to the Fc fragment of trastuzumab.

### Example 2

### Biacore Evaluation of VH3 Binding Polypeptides with Improved Alkali Clean Stability

### Summary

Based on the results from the Biacore evaluation of VH3 binding polypeptides (SEQ ID NO:1-4) as set out in Example 1, eight additional VH3 binding polypeptides (Fr7-14; SEQ ID NO:5-12) were identified with a diversity of amino acids at seven positions (9, 10, 11, 13, 14, 17 and 18) as set out in Table 3. The polypeptides were evaluated in the following aspects: trastuzumab Fc binding, trastuzumab binding and alkali clean stability.

TABLE 3
Summary of mutations (SEQ ID NO: 5 to 12)

| VH3 binding | SEQ | Position | | | | | | |
|---|---|---|---|---|---|---|---|---|
| polypeptide | ID NO: | 9 | 10 | 11 | 13 | 14 | 17 | 18 |
| Fr7 | 5 | A | Q | E | L | A | | |
| Fr8 | 6 | A | Q | R | L | A | | |
| Fr9 | 7 | A | Y | R | L | A | | |
| Fr10 | 8 | A | Y | R | L | W | | |
| Fr11 | 9 | A | Y | R | L | W | A | R |
| Fr12 | 10 | A | Y | R | L | A | A | R |
| Fr13 | 11 | A | Y | R | L | A | H | L |
| Fr14 | 12 | A | Y | R | L | W | H | L |

The polypeptide variants (SEQ ID NO:5-12) were evaluated together with an alkali clean stabile positive control that binds both Fc and Fab fragments of trastuzumab (SEQ ID NO:88) and a non-Fc-binding control that is not alkali clean stabile (Negative CTRL).

The following aspects were evaluated:
(1) Affinity assessment of Fc interaction (tested through high concentration injection of Fc fragment generated from trastuzumab).
(2) Affinity assessment of trastuzumab interaction (tested through high concentration injection of trastuzumab)
(3) Alkali clean stability (tested by reduction of trastuzumab binding after increasing number of treatments with 0.5 M NaOH).

The inventors surprisingly found additional novel VH3 binding polypeptides (SEQ ID NO:5-12) with a diversity of amino acids at positions 9, 10, 11, 13, 14 and optionally at 17 and 18 that show binding to trastuzumab, exhibit significantly reduced or abolished affinity for the Fc region of trastuzumab and are alkali clean stabile.

### Materials and Methods

Generation of his-Purified Candidate Polypeptides

Materials and equipment used were as follows: glycerol stocks for SEQ ID NO:5 to 12; LB culture medium, 2YT culture medium, Carbenicillin, IPTG 1 M, 125 ml & 250 ml baffled glass shake flasks, Infors HT Shaking incubator; His-GraviTrap™ and kit (Cytiva™), PD10 GraviTraps (Cytiva™)

5 μl glycerol stock of each variant was inoculated in 4 ml LB supplemented with 200 μg/ml carbenicillin in 125 ml flasks and incubated at 37° C. overnight at 200 rpm.

Protein expression culture medium (2YT medium supplemented with 100 μg/ml carbenicillin) was prepared and added to filled baffled shake flasks (50 ml per flask, 20 flasks in total). Each flask was inoculated with approximately 500 μl from the previous overnight culture to obtain a starting OD600 of 0.05. The flasks were incubated in Infors HT shaking incubator for approximately 2.5 hours at 37° C. and 130 rpm shaking until OD600 reached 0.7. Thereafter 25 μl IPTG (1 M) was added and the flasks were incubated in Infors HT shaking incubator overnight at 27° C. and 130 rpm shaking, whereafter the cultures were pelleted (8000 g, 5 min) and re-suspended in 10 ml PBS+20 mM imidazole in Falcon tubes. To generate crude variant lysates the Falcon tubes were incubated in 80-85° C. water bath for 10 minutes followed by pelleting of cell debris by centrifugation at 10000 g for 10 min and filtration of supernatant with a 0.2 μm filter.

The samples were purified using His GraviTraps and kit according to kit instructions (PBS+20 mM imidazole was used as binding/wash buffer). Buffer exchange into PBS was performed using PD10 Gravitraps and kit instructions.

Concentration of buffer exchanged samples was measured using NanoDrop (Thermo Scientific) according to manufacturer's instructions. Following purification, samples were kept in refrigerator until all measurements performed and were then transferred to eppendorf tubes and kept in refrigerator.

### Biacore Analysis of Binding Affinity for Fc Fragment of Trastuzumab and Trastuzumab

To assess binding to the Fc fragment of trastuzumab and trastuzumab, the VH3 binding polypeptides were immobilized on a Biacore CM5 chip. Trastuzumab Fc fragment (5000 nM) and trastuzumab (625 nM) were used as analyte.

pH-Scouting for Immobilization Conditions:

Materials and equipment used were as follows: VH3 binding polypeptide variants (SEQ ID NO:5-12); Biacore CM5 chips (Cytiva™); Biacore Acetate-buffers (Cytiva™) with different pH; Biacore™ 8K+ instrument (Cytiva™).

The pH-scouting was performed on Biacore™ 8K+ using the standard method defined in the software at different pH values (pH 4.5, 5, 5.5). A pH value corresponding to one unit below the pI of the target ligand was found to give the best surface attraction.

### Biacore Binding Analysis:

Materials and equipment used were as follows: CM5 sensor chips (Cytiva™) Biacore™ NHS coupling kit (Cytiva™), VH3 binding polypeptides (SEQ ID NO:5 to 12); Biacore™ 8K+(Cytiva™); Fc from trastuzumab (SEQ ID NO:184); trastuzumab (SEQ ID NO:178 and 179).

Immobilization was performed using a standard method in Biacore software with coupling of VH3 binding polypeptide variants in FC2 and activation/inactivation in FC1. VH3 binding polypeptide variants were diluted in an appropriate pH (based on pH-scouting) in acetate buffer at a concentration ranging from 10-30 μg/ml. The immobilization levels did vary somewhat between different polypeptide variants (approx. 600-1300 Ru).

In each run the polypeptide was immobilized in FC2. Multiple chips were used until all candidates were tested.

Biacore method for binding analysis:
Running buffer: PBS-P+
Flow rate: 10 μl/min
Sample injection: 600 s/10 min over both Flow Cells (FC1 and FC2)
Dissociation time: 600 s/10 min
Regeneration: 10 mM Glycin-HCl pH 1.5, 30 μl/min, 2×30 s

Injections of analyte (trastuzumab or Fc fragment of trastuzumab) for each channel (cycles) were as follows: buffer, 156 nM, 313 nM, 625 nM, 1250 nM, 2500 nM, 5000 nM.

All sensorgrams were generated as reference subtracted and the responses as the difference between baseline before injection and signal just before end of injection.

### Biacore Analysis of Alkali Clean Stability

Assessment of alkali clean stability was done by immobilizing the VH3 binding polypeptides (SEQ ID NO:5-12) on a CM5 Biacore™ chip followed by 100 repeating cycles of binding to trastuzumab (1 μM) followed by injection of NaOH (0.5 M).

Materials and equipment used were as follows: CM5 sensor chips (Cytiva™) Biacore™ NHS coupling kit (Cytiva™), VH3 binding polypeptides (SEQ ID NO:5-12); Biacore™ 8K+(Cytiva™); trastuzumab; 0.5 M NaOH.

Immobilization was performed using a standard method in Biacore software with coupling of VH3 binding polypeptide variants in FC2 and activation/inactivation in FC1. VH3 binding polypeptide variants were diluted in an appropriate pH (based on pH scouting) in acetate buffer at a concentration ranging from 10-30 μg/ml. The immobilization levels did vary somewhat between different polypeptide variants (approx. 600-1300 Ru).

Biacore method for alkaline stability (per cycle):
Running buffer: PBS-P+
Flow rate: 10 μl/min
Sample injection 1 (trastuzumab 1 μM): 300 s over both Flow Cells
Dissociation time 1: 60 s
Sample injection 2 (0.5 M NaOH): 600 s over both flow cells.
Regeneration: 10 mM Glycin-HCl pH 1.5, 30 μl/min, 2×30 s

This cycle was repeated more than 100 times to follow stability of trastuzumab response values.

All sensorgrams were generated as reference subtracted and the responses as the difference between baseline before injection and signal just before end of injection.

### Results

### Biacore Analysis of Binding Affinity for Fc Fragment of Trastuzumab and Trastuzumab

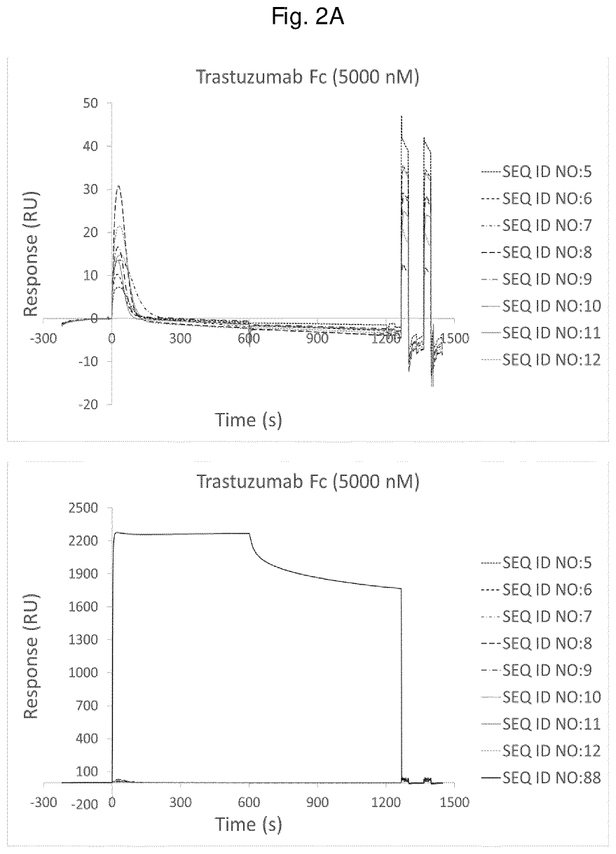

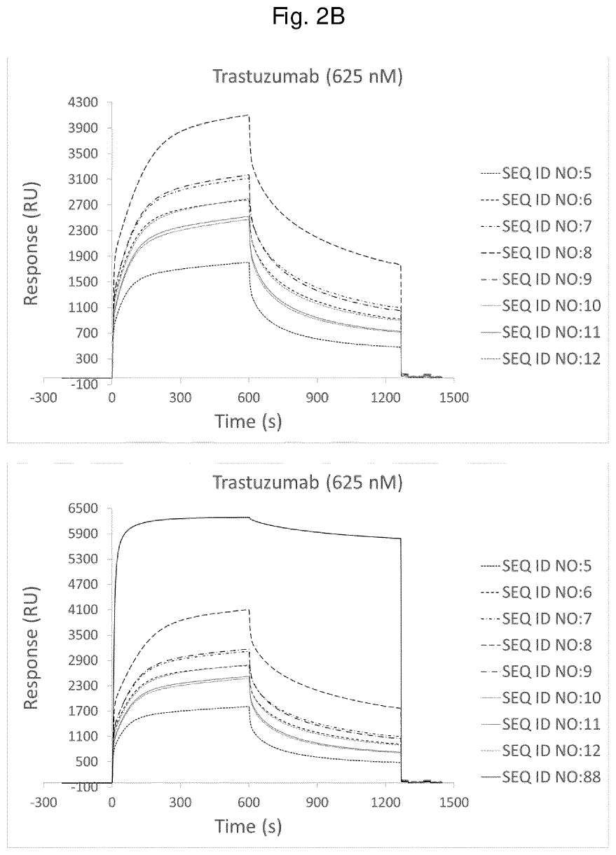

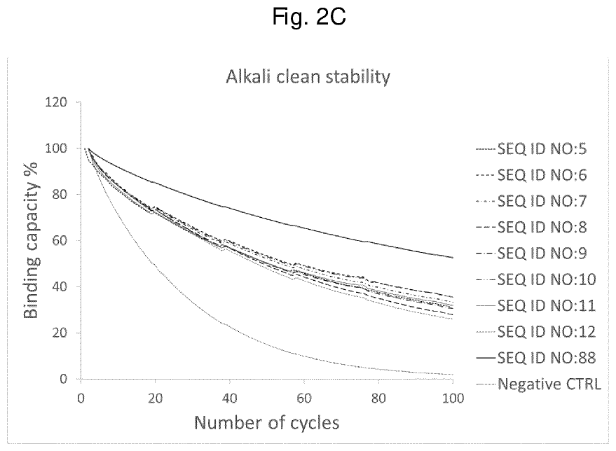

The response levels of the Fc fragment of trastuzumab and the trastuzumab interactions with the VH3 binding polypeptide variants (SEQ ID NO:5 to 12) are depicted in FIG. 2A and FIG. 2B. The data show that the novel VH3 binding polypeptide variants (SEQ ID NO:5 to 12) exhibit a significantly reduced or abolished binding affinity for Fc fragment of trastuzumab while retaining binding to trastuzumab. The positive reference (SEQ ID NO:88) shows binding to both Fc and Trastuzumab with >2000 RU and >6000 RU at injection, respectively.

### Biacore Analysis of Alkali Clean Stability

Alkali clean stability was visualized by plotting normalized trastuzumab response values (in % of response in cycle 2) from the second cycle as set out above to the last cycle run. The alkali clean stabile positive control that binds both Fc and Fab fragments (SEQ ID NO:88) and a non-Fc-binding control that is not alkali clean stabile (Negative CTRL) were present as controls in each separate stability run. Trastuzumab response values after 100 cleaning cycles are summarized in Table 4. FIG. 2C shows plots of alkali clean stability for the tested VH3 binding polypeptide variants (SEQ ID NO:5 to 12) in comparison to the alkali clean stabile positive control that binds both Fc and Fab fragments (SEQ ID NO:88) and a non-Fc-binding control that is not alkali clean stabile (Negative CTRL). In each cycle, 0.5 M NaOH was injected with a contact time of 10 min at 10 μl/min.

TABLE 4
Trastuzumab response values (in % of
response in cycle 2) after 100 cycles

| | | Binding % after | |
|---|---|---|---|
| Polypeptide | SEQ ID NO | 100 cycles | RU cycle 2 |
| | 88 | 52.6 | 3597 |
| Fr8 | 6 | 35.6 | 862 |
| Fr12 | 10 | 35.5 | 732 |
| Fr9 | 7 | 33.4 | 931 |
| Fr13 | 11 | 32 | 750 |
| Fr7 | 5 | 31.5 | 541 |
| Fr11 | 9 | 30.6 | 1013 |
| Fr10 | 8 | 27.9 | 1360 |
| Fr14 | 12 | 26 | 910 |
| Negative CTRL | | 2 | 3322 |

### Conclusions

Additional novel VH3 binding polypeptides (SEQ ID NO:5-12) were identified that show no binding to the Fc fragment of trastuzumab and show binding to trastuzumab.

SEQ ID NO:5-12 are alkali clean stabile and retain at least 26% of their trastuzumab response (binding %) in cycle 2, after 100 cleaning cycles of 0.5 M NaOH with a contact time of 10 min at 10 μl/min.

It is concluded that at least said combinations of amino acids at positions 9, 10, 11, 13, 14 and optionally 17 and 18 as set out in Table 3, lead to the advantageous properties of VH3 binding polypeptides according to the present invention herein: SEQ ID NO:5-12 are alkali clean stabile, retain binding to trastuzumab and exhibit no binding to the Fc fragment of trastuzumab.

Using the information from the alkali clean stability assay together with the amino acid sequence information, it is concluded that a scaffold with the motif AQR-LA in positions X₉X₁₀X₁₁-X₁₃X₁₄ and a scaffold with the motif AYR-LA-AR in positions X₉X₁₀X₁₁-X₁₃X₁₄-X₁₇X₁ₐ exhibit particularly desirable alkali clean stability. A VH3 binding polypeptide comprising a scaffold with the motif AYR-LA-AR in positions X₉X₁₀X₁₁-X₁₃X₁₄-X₁₇X₁ₐ has an additional advantageous property that the presence of tyrosine (Y in position 10) within the sequence permits the measurement of the concentration of said VH3 binding polypeptide in a spectrophotometer.

### Example 3

### Biacore Evaluation of VH3 Binding Polypeptides with Enhanced Binding Affinity for Trastuzumab Fab

### Summary

Based on the results from the Biacore evaluation of the additional VH3 binding polypeptides (SEQ ID NO:5-12) as set out in Example 2, three additional VH3 binding polypeptide variants were identified (Fr31-33; SEQ ID NO:13 to 15) with a diversity of amino acids at nine positions (9, 10, 11, 13, 14, 17, 18, 28 and 29) as set out in Table 5. The polypeptides were evaluated in the following aspects: trastuzumab Fc binding, trastuzumab Fab binding, trastuzumab binding and alkali clean stability.

TABLE 5
Summary of mutations (SEQ ID NO: 13 to 15)

| VH3 binding | SEQ | Position | | | | | | | | |
|---|---|---|---|---|---|---|---|---|---|---|
| polypeptide | ID NO: | 9 | 10 | 11 | 13 | 14 | 17 | 18 | 28 | 29 |
| Fr31 | 13 | A | Y | R | L | A | A | R | N | G |
| Fr32 | 14 | A | Y | R | L | A | A | R | A | A |
| Fr33 | 15 | A | Y | R | L | A | A | R | A | G |

The polypeptide variants SEQ ID NO:13-15 were evaluated together with an alkali clean stabile positive control that binds both Fc and Fab fragments (SEQ ID NO:88) and a non-Fc-binding control that is not alkali clean stabile (Negative CTRL).

The following aspects were evaluated:
(1) Affinity assessment of Fc interaction (tested through high concentration injection of Fc fragment generated from trastuzumab).
(2) Affinity assessment of Fab interaction (tested through high concentration injection of Fab fragment generated from trastuzumab)
(3) Affinity assessment of trastuzumab interaction (tested through high concentration injection of trastuzumab)
(4) Alkali clean stability (tested by reduction of trastuzumab binding after increasing number of treatments with 0.5 M NaOH).

The inventors surprisingly found additional novel VH3 binding polypeptides (SEQ ID NO:13 to 15) with a diversity of amino acids at nine positions (positions 9, 10, 11, 13, 14, 17, 18, 28 and 29) that exhibit even more desirable properties: are alkali clean stabile, show binding to trastuzumab and to the Fab fragment of trastuzumab but exhibit no affinity for the Fc region of trastuzumab.

### Materials and Methods

Generation of his-Purified Candidate Polypeptides

Materials and equipment used were as follows: glycerol stocks for SEQ ID NO:13 to 15; LB culture medium, 2YT culture medium, Carbenicillin, IPTG 1 M, 125 ml & 250 ml baffled glass shake flasks, Infors HT Shaking incubator; His-GraviTrap™ and kit (Cytiva™), PD10 GraviTraps (Cytiva™)

5 μl glycerol stock of each variant was inoculated in 4 ml LB supplemented with 200 μg/ml carbenicillin in 125 ml flasks and incubated at 37° C. overnight at 200 rpm.

Protein expression culture medium (2YT medium supplemented with 100 μg/ml carbenicillin) was prepared and added to filled baffled shake flasks (50 ml per flask, 20 flasks in total). Each flask was inoculated with approximately 500 μl from the previous overnight culture to obtain a starting OD600 of 0.05. The flasks were incubated in Infors HT shaking incubator for approximately 2.5 hours at 37° C. and 130 rpm shaking until OD600 reached 0.7. Thereafter 25 μl IPTG (1 M) was added and the flasks were incubated in Infors HT shaking incubator overnight at 27° C. and 130 rpm shaking, whereafter the cultures were pelleted (8000 g, 5 min) and re-suspended in 10 ml PBS+20 mM imidazole in Falcon tubes. To generate crude variant lysates the Falcon tubes were incubated in 80-85° C. water bath for 10 minutes followed by pelleting of cell debris by centrifugation at 10000 g for 10 min and filtration of supernatant with a 0.2 μm filter.

The samples were purified using His GraviTraps and kit according to kit instructions (PBS+20 mM imidazole was used as binding/wash buffer). Buffer exchange into PBS was performed using PD10 Gravitraps and kit instructions.

Concentration of buffer exchanged samples was measured using NanoDrop (Thermo Scientific) according to manufacturer's instructions. Following purification, samples were kept in refrigerator until all measurements performed and were then transferred to eppendorf tubes and kept in refrigerator.

### Biacore Analysis of Binding Affinity for Fc and Fab Fragments of Trastuzumab and Trastuzumab

To assess binding to trastuzumab and to the Fc and Fab fragments of trastuzumab, the VH3 binding polypeptides (SEQ ID NO:13 to 15) were immobilized on a Biacore™ CM5 chip. Trastuzumab Fc fragment (5000 nM), trastuzumab Fab fragment (2500 nM) and trastuzumab (625 nM) were used as analyte.

pH-Scouting for Immobilization Conditions:

Materials and equipment used were as follows: VH3 binding polypeptide variants (SEQ ID NO: 13 to 15); Biacore™ CM5 chips (Cytiva™); Biacore Acetate-buffers (Cytiva™) with different pH; Biacore™ 8K+ instrument (Cytiva™)

The pH-scouting was performed on Biacore™ 8K+ using the standard method defined in the software at different pH values (pH 4.5, 5, 5.5). A pH value corresponding to one unit below the pI of the target ligand was found to give the best surface attraction.

### Biacore Binding Analysis:

Materials and equipment used were as follows: CM5 sensor chips (Cytiva™) Biacore™ NHS coupling kit (Cytiva™), VH3 binding polypeptides (SEQ ID NO:13 to 15); Biacore™ 8K+(Cytiva™); Fab (prepared from trastuzumab in-house), Fc from trastuzumab, trastuzumab

Immobilization was performed using a standard method in Biacore software with coupling of VH3 binding polypeptide variants in FC2 and activation/inactivation in FC1. VH3 binding polypeptide variants were diluted in an appropriate pH (based on pH-scouting) in acetate buffer at a concentration ranging from 10-30 μg/ml. The immobilization levels did vary somewhat between different polypeptide variants (approx. 600-1300 Ru).

In each run the polypeptide was immobilized in FC2. Multiple chips were used until all candidates were tested.

Biacore method for binding analysis:
Running buffer: PBS-P+
Flow rate: 10 μl/min
Sample injection: 600 s/10 min over both Flow Cells (FC1 and FC2)
Dissociation time: 600 s/10 min
Regeneration: 10 mM Glycin-HCl pH 1.5, 30 μl/min, 2×30 s

Injections of analyte (Fab or Fc fragment of trastuzumab or trastuzumab) for each channel (cycles) were as follows: buffer, 156 nM, 313 nM, 625 nM, 1250 nM, 2500 nM, 5000 nM.

All sensorgrams were generated as reference subtracted and the responses as the difference between baseline before injection and signal just before end of injection.

### Biacore Analysis of Alkali Clean Stability

Assessment of alkali clean stability was done by immobilizing the VH3 binding polypeptides (SEQ ID NO:13 to 15) on a CM5 Biacore chip followed by 100 repeating cycles of binding to trastuzumab (1 μM) followed by injection of NaOH (0.5 M).

Materials and equipment used were as follows: CM5 sensor chips (Cytiva™) Biacore™ NHS coupling kit (Cytiva™), VH3 binding polypeptides (SEQ ID NO:13 to 15); Biacore™ 8K+(Cytiva™); trastuzumab; 0.5 M NaOH.

Immobilization was performed using a standard method in Biacore software with coupling of VH3 binding polypeptide variants in FC2 and activation/inactivation in FC1. VH3 binding polypeptide variants were diluted in an appropriate pH (based on pH scouting) in acetate buffer at a concentration ranging from 10-30 μg/ml. The immobilization levels did vary somewhat between different polypeptide variants (approx. 600-1300 Ru).

Biacore method for alkaline stability (per cycle):
Running buffer: PBS-P+
Flow rate: 10 μl/min
Sample injection 1 (trastuzumab 1 μM): 300 s over both Flow Cells
Dissociation time 1: 60 s
Sample injection 2 (0.5 M NaOH): 600 s over both flow cells.
Regeneration: 10 mM Glycin-HCl pH 1.5, 30 μl/min, 2×30 s

This cycle was repeated more than 100 times to follow stability of trastuzumab response values.

All sensorgrams were generated as reference subtracted and the responses as the difference between baseline before injection and signal just before end of injection.

### Results

### Biacore Analysis of Binding Affinity for Fc and Fab Fragments of Trastuzumab and Trastuzumab

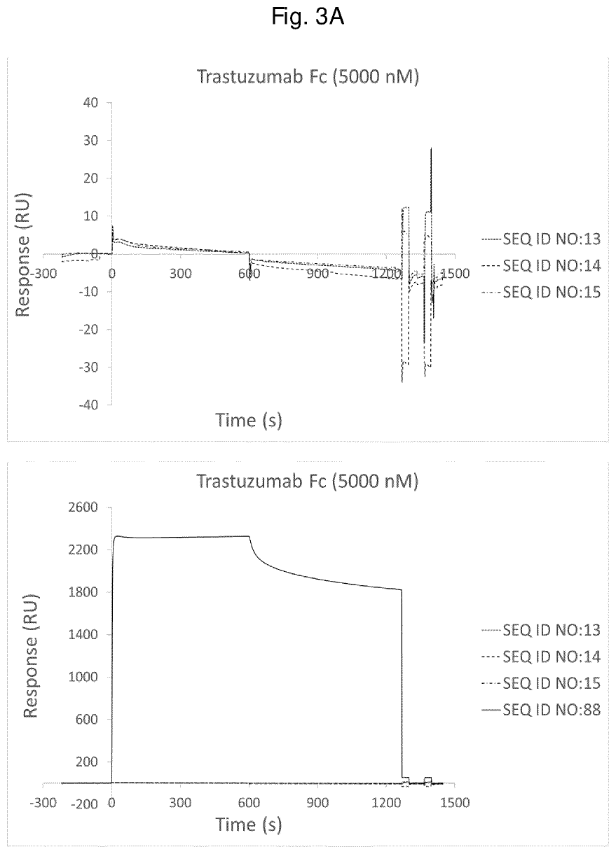

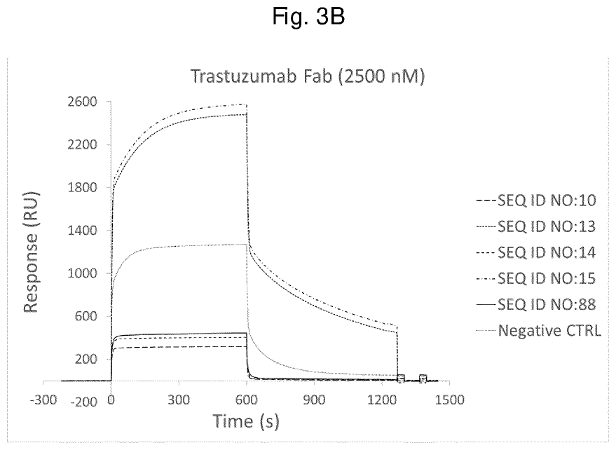

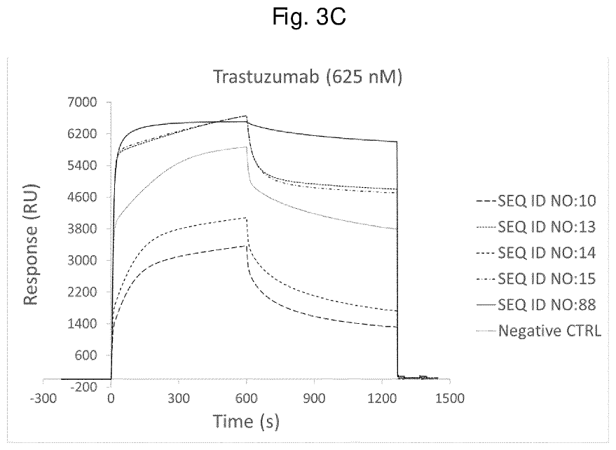

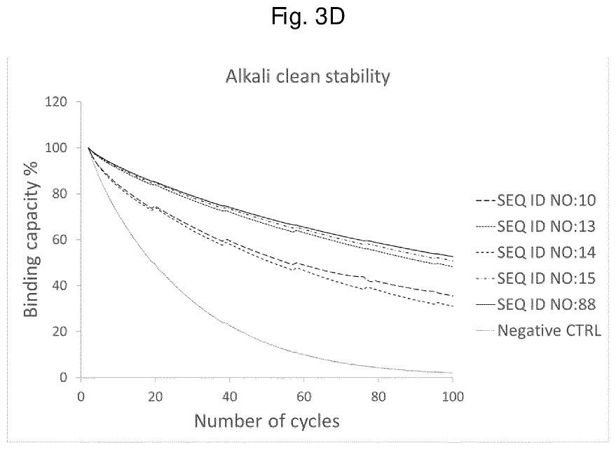

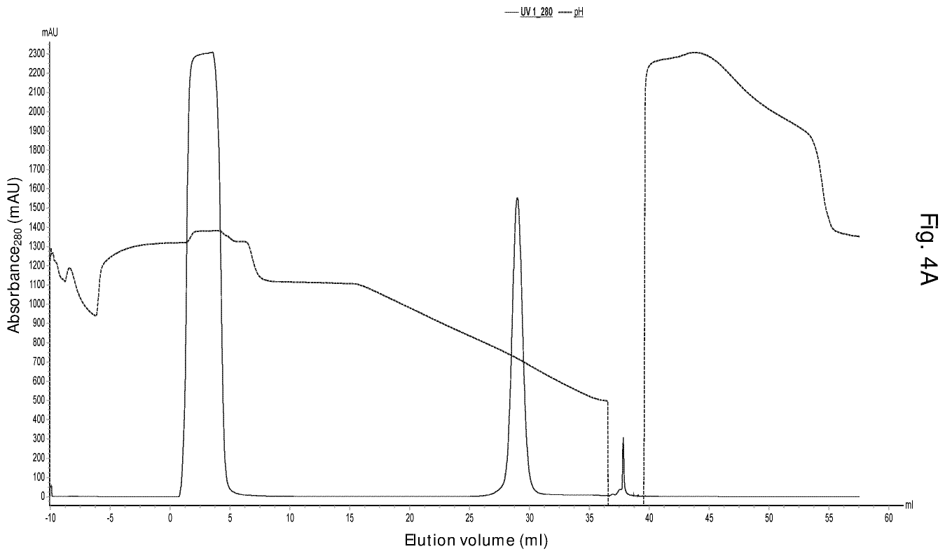

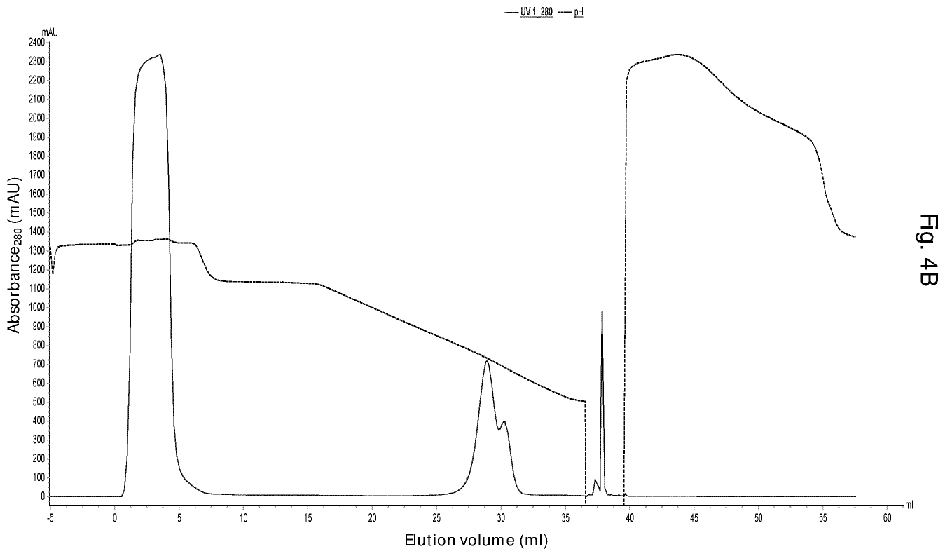

The response levels of the trastuzumab Fc, trastuzumab Fab and trastuzumab interactions with the VH3 binding polypeptide variants (SEQ ID NO:13 to 15) are depicted in FIG. 3A, FIG. 3B and FIG. 3C. The results show that the VH3 binding polypeptide variants (SEQ ID NO:13-15) exhibit a significantly reduced or abolished binding affinity for the Fc fragment of trastuzumab while retaining binding to trastuzumab and to the Fab fragment of trastuzumab. The alkali clean stabile positive control that binds both Fc and Fab fragments of trastuzumab (SEQ ID NO:88) shows binding to trastuzumab Fc, trastuzumab Fab and trastuzumab with >2000 RU, >300 RU and >6000 RU at injection, respectively.

The three additional VH3 binding polypeptides SEQ ID NO:13 to 15 show improved binding to trastuzumab and trastuzumab Fab in comparison to the VH3 binding polypeptide (SEQ ID NO:10) tested in Example 2. SEQ ID NO:13 and SEQ ID NO:15 show improved binding to both Fab and trastuzumab in comparison to the non-Fc-binding control that is not alkali clean stabile (Negative CTRL). SEQ ID NO:13 and SEQ ID NO:15 show improved binding to Fab in comparison to the alkali clean stabile positive control SEQ ID NO:88 that binds both Fc and Fab fragments. SEQ ID NO:13 and SEQ ID NO:15 show comparable trastuzumab response values to that of the positive control SEQ ID NO:88.

### Biacore Analysis of Alkali Clean Stability

Stability was visualized by plotting normalized trastuzumab response values (in % of response in cycle 2) from the second cycle as set out above to the last cycle run. An alkali clean stabile positive control that binds both Fc and Fab fragments of trastuzumab (SEQ ID NO:88) and a non-Fc-binding control that is not alkali clean stabile (Negative CTRL) were present as controls in each separate stability run. Trastuzumab response values after 100 cleaning cycles are summarized in Table 6. FIG. 3D shows plots of alkali clean stability for the tested VH3 binding polypeptide variants (SEQ ID NO:13-15) in comparison to the alkali clean stabile positive control that binds both Fc and Fab fragments of trastuzumab (SEQ ID NO:88) and a non-Fc-binding control that is not alkali clean stabile (Negative CTRL). In each cycle, 0.5 M NaOH was injected with a contact time of 10 min at 10 μl/min.

SEQ ID NO:14 has an alkali clean stability comparable to that of the VH3 binding polypeptide SEQ ID NO:10 tested in Example 2. SEQ ID NO:13 and SEQ ID NO:15 have improved alkali clean stability in comparison to that of the VH3 binding polypeptide SEQ ID NO:10. SEQ ID NO:13 and SEQ ID NO:15 has an alkali clean stability comparable to that of the alkali clean stabile positive control that binds both Fc and Fab fragments (SEQ ID NO:88). The VH3 binding polypeptides (SEQ ID NO:13 to 15) have a greater alkali clean stability than that of the non-Fc-binding control that is not alkali clean stabile (Negative CTRL).

TABLE 6
Trastuzumab response values (in % of
response in cycle 2) after 100 cycles

| | | Binding % after | |
|---|---|---|---|
| Polypeptide | SEQ ID NO | 100 cycles | RU cycle 2 |
| | 88 | 52.6 | 3597 |
| Fr33 | 15 | 50.7 | 3633 |
| Fr31 | 13 | 48.3 | 3513 |
| Fr32 | 14 | 31 | 1820 |
| Negative CTRL | | 2 | 3322 |

### Conclusions

The additional novel VH3 binding polypeptides (SEQ ID NO:13 to 15) show no binding to trastuzumab Fc. The additional novel VH3 binding polypeptides (SEQ ID NO:13 to 15) show improved binding to both Fab and trastuzumab in comparison to the VH3 binding polypeptide (SEQ ID NO:10) tested in Example 2.

Additional VH3 binding polypeptides (SEQ ID NO:13 to 15) are alkali clean stabile and retain at least 31% of their trastuzumab response in cycle 2, after 100 cleaning cycles of 0.5 M NaOH with a contact time of 10 min at 10 μl/min. SEQ ID NO:14 has an alkali clean stability comparable to that of the VH3 binding polypeptide SEQ ID NO:10. SEQ ID NO:13 and SEQ ID NO:15 have an improved alkali clean stability in comparison to that of the VH3 binding polypeptide SEQ ID NO:10. SEQ ID NO:13 and SEQ ID NO:15 show improved binding to trastuzumab Fab in comparison to the alkali clean stabile positive control that binds both Fc and Fab fragments of trastuzumab (SEQ ID NO:88).

It is concluded that at least the presence of amino acids at positions 9, 10, 11, 13, 14, 17, 18, 28 and 29 as set out in Table 5, leads to the advantageous properties of VH3 binding polypeptides according to the present invention herein: said VH3 binding polypeptides SEQ ID NO:13-15 exhibiting even more improved alkali clean stability properties, exhibit binding to the Fab fragment but show no binding to the Fc fragment of trastuzumab.

Based on the sequence information shown in Table 5 and the results presented in Example 3, it is concluded that VH3 binding polypeptides comprising a glycine (G) in position 29 (SEQ ID NO:13 and 15) show a surprisingly high binding affinity for trastuzumab and for the Fab fragment of trastuzumab in comparison to a VH3 binding polypeptide that comprises an alanine (A) in position 29 (SEQ ID NO: 14) as well as in comparison to the alkali clean stabile positive control that binds both Fc and Fab fragments of trastuzumab (SEQ ID NO:88). It is further concluded that a VH3 binding polypeptide comprising an alanine (A) in position 28 and a glycine (G) in position 29 (SEQ ID NO:15) has surprisingly superior characteristics in alkali clean stability in comparison to a VH3 binding polypeptide comprising an asparagine (N) in position 28 and a glycine (G) in position 29.

### Example 4

### Evaluation of VH3 Binding Polypeptide Multimer Variants by Chromatography

### Summary

A subset of alkali clean stabile VH3 binding polypeptides with abolished Fc binding (Fr32 and Fr33; SEQ ID NO:14 and 15) were chosen for further studies on column. The further studies included multimerization of the polypeptides to tetramers and hexamers and expression of said multimers in E. coli. On these multimeric VH3 binding polypeptides (SEQ ID NO:175 to 177), a primary characterization was performed, wherein the ligands were evaluated for dynamic binding capacity (DBC), pH elution and alkali clean stability.

The effect of A29G mutation on tetramers of VH3 binding polypeptides (SEQ ID NO:175 and 176) was investigated for DBC and elution pH in a pH gradient using trastuzumab. A hexamer of a VH3 binding polypeptide (SEQ ID NO:177) was tested for DBC and elution pH in a pH gradient using trastuzumab and different target molecules. Moreover, alkali clean stability of SEQ ID NO:177 was tested by running 4h incubations with 0.5 M NaOH followed by DBC measurements. A hexamer (SEQ ID NO: 181) of an alkali clean stabile polypeptide that binds to both the Fc and Fab fragments of trastuzumab (SEQ ID NO: 88) was used as control.

### Materials and Methods

### Plasmids and Cell-Banks

A subset of H6C-tagged multimer candidate variants were prepared according to a general formula. In particular, SEQ ID NO:14 as tetramers and SEQ ID NO:15 as tetramers and hexamers, which comprise the sequences according to SEQ ID NO:175 to 177, respectively, were ordered as DNA synthesis plasmids from ATUM (Newark, CA, USA). The plasmids were transformed into chemically competent E. coli K12-017 cells and research cell-banks were made according to standard procedures.

### Fermentation

Fermentation was performed with the abovementioned multimer candidates with consecutive mid-stream steps.

Seed cultures were prepared in 500 mL baffled shake flasks with 100 mL Terrific Broth (TB), 100 μL kanamycin (50 mg/mL) and 100 μL research cell bank (RCB) of SEQ ID NO:175-177. The seed cultures were incubated for 15 h, at 37° C. and 180 rpm agitation to final OD600 values around 20. Fermentation medium was prepared and sterilized in the reactor for 20 minutes at 121° C. followed by the addition of additives. Reactor was inoculated with an OD600 of 0.2. The starting conditions for the cultivation were 37° C., pH 7.1, 300 RPM stirring, 1 L/min aeration and DO was kept constant at 30%. Glucose feed with specific growth rate of 0.3/h was started when the glucose level in the reactor was below 1 g/L. Protein expression was induced using Isopropyl b-D-thiogalactopyranoside (IPTG) with a final concentration of 1 mM. Cultivation was stopped after 25 hours, and heat treated in the reactor.

### Clarification of Heat-Treated Fermentation Medium

The heat-treated fermentation medium was clarified using a ÅKTA flux system connected to a 3600 cm² hollow fibre filter cartridge with a 0.2 μM cut-off (Cytiva™ Uppsala, Sweden). Fermentation broth was concentrated 3 times followed by diafiltration using water. Retentate was concentrated until pressure reached 2 bar. Na-azide was added to the harvested material to a final concentration of about 0.02% before storage in a cold room. The clarified feed material was loaded onto the first chromatography step.

### Chromatographic Purification

Constructs were synthesized with a hexa histag and were purified with Ni-sepharose 6FF (Cytiva™, Uppsala, Sweden). Clarified feed was loaded onto a Ni sepharose 6FF resin packed in HiScale26/40 column. The column was equilibrated with 50 mM phosphate, 500 mM NaCl, pH 7.5 followed by load of 300 mL clarified feed. Purified protein was eluted in a step gradient using 50 mM phosphate, 500 mM NaCl, pH 7.5 with 2 M imidazole (Merck, Damstadt, Germany). Following capture on His-tag, the proteins were further purified on Source 15Q. The eluate of capture step was reduced for 60 min at room temperature by adding a final concentration of 50 mM DTT. After reduction the sample was desalted to 25 mM Tris pH8 using Sephadex G50 (Cytiva™, Uppsala, Sweden) followed by loading onto Source 15 Q (Cytiva™, Uppsala, Sweden). The protein was eluted off the resin in a salt gradient with 25 mM Tris pH 8, 500 mM NaCl. The final protein was cleared from E. coli HCP, aggregates, and truncated versions with the polishing step. The eluted protein was concentrated to a high concentration of approximately 50 g/L using Vivaspin 20 (Cytiva™, Uppsala, Sweden) spin-columns with 5 kDa cutoff. Concentrated protein was used for coupling to base matrix.

### Coupling

Coupling was made on 60 μm and 50 μm agarose beads using epoxy chemistry. Protein ligand was reduced in a 50 ml Falcon tube. To 20 mL of ligand solution (50 mg/mL), 169 mg NaHCO₃, 21 mg Na2CO₃, 175 mg NaCl and 7 mg EDTA, was added. The Falcon tube was placed on a rollertable for 5-10 min, followed by addition of 77 mg of DTT. Reduction proceeded for >45 min. The ligand solution was then desalted using a PD10 column packed with Sephadex G-25 (Cytiva, Uppsala, Sweden). The ligand content in the desalted solution was determined by measuring 276 nm UV absorption. The epoxy activated gel was washed with 3-5 gel volumes (GV) 0.1 M phosphate, 1 mM EDTA pH 8.6 and the ligand was then coupled according to the method described in U.S. Pat. No. 6,399,750. After immobilization, the resins were washed 3×GV with distilled water. The resins and 1 GV of 0.1 M phosphate, 1 mM EDTA, 10% thioglycerol pH 8.6 was mixed and the tubes were left in a shaking table at room temperature over-night. Resins were then washed alternately with 3×GV 0.1 M TRIS, 0.15 M NaCl pH 8.6 and 0.5 M HAc followed by 8-10×1GV with distilled water. Resin samples were sent for amino acid analysis and the ligand density (LD) was calculated from the total amino acid content in mg/mL resin.

Column Packing, Sampling with 1 mL Cube

In order to pack same volume of resin each time a 50% resin slurry was prepared and packed in 1 mL cube using a set vacuum. The 1 ml cubed resin sample was transferred to a Tricorn column (Cytiva™, Uppsala, Sweden) using a Pasteur pipette.

### Control of Column Packing

The packing of the columns was checked by injecting a solution of 0.8M NaCl through the columns and calculating the asymmetry of the resulting peak. The column was equilibrated with 2 CV 0.4 M NaCl with a flow rate of 1 mL/min. 10 μl of the 0.8 M NaCl solution was injected at a flow rate of 20 cm/h. The plate height and peak asymmetry factor was then evaluated using the ÅKTA™ system (Cytiva™, Uppsala, Sweden).

### Preparation of Trastuzumab

Purified mAb solution was diluted in PBS (Medicago, Uppsala, Sweden) and was filtered with a 0.2 μm syringe filter. The concentration was determined by measuring the max UV at 280 nm on an ÅKTA Pure25 system (Cytiva™, Uppsala, Sweden).

### DBC Measurement

DBC measurements were made in Tricorn 5/50 columns. The column was equilibrated with PBS buffer. Trastuzumab, trastuzumab Fab fragment or nanobody was loaded to the columns via the sample pump at desired flow rate typically 0.166 mL/min to achieve 6 min residence time (depending on the bed height of the column) until the UV signal of approx. 20% of maximum was reached. The column was then washed with PBS buffer at flow rate 1 mL/min. The protein was eluted with elution buffer (50 mM sodium citrate pH 2.5) at flow rate 1 mL/min. The column was cleaned with 0.1 M NaOH, at a flow rate of 0.166 mL/min for 15 min followed by re-equilibration with PBS buffer.

pH-Elution Using a Gradient

pH elution measurements were made in Tricorn 5/50 columns. The column was equilibrated with PBS buffer. Approximately 10 mg Trastuzumab, Trastuzumab Fab fragment or nanobody was loaded to the columns via the sample pump at desired flow rate typically 0.166 mL/min to achieve 6 min residence time. The column was then washed with PBS buffer at flow rate 1 mL/min. The protein was eluted with an elution gradient from 50 mM sodium citrate pH 6.5 to 50 mM sodium citrate pH 2.5 at flow rate 1 mL/min. The column was cleaned with 0.1 M NaOH, at a flow rate of 0.166 mL/min for 15 min followed by re-equilibration with PBS buffer. The elution pH is determined as the apex of the elution peak.

### Accelerated NaOH Studies

Accelerated NaOH studies were performed by running a start DBC run to measure resin capacity at a set residence time followed by a fill up of the column with 0.5 M NaOH. When the column was completely filled, the flow was stopped, and the column was incubated in NaOH for 4 h followed by re-equilibration with PBS and a new DBC measurement. This iteration was continued until the column had been incubated for a total of 24 h in 0.5 M NaOH.

### Results

### Evaluation VH3 Binding Polypeptide Tetramers by Chromatography

Tetrameric variants (SEQ ID NO:175 and SEQ ID NO:176) were evaluated with DBC measurements and pH elution. The results are summarized in Table 7.

TABLE 7
Reference resin is SEQ ID NO: 181. The two test resins SEQ ID
NO: 175 and SEQ ID NO: 176 exhibit only VH3 interaction. DBC
was measured with trastuzumab at 6 min residence time (RT)
and 10% break-through. The pH elution is measured with approximately
10 mg trastuzumab Ligand density (LD) is shown.

| | | | DBC | |
|---|---|---|---|---|
| Multimeric | SEQ ID | LD | trastuzumab | Elution |
| variant | NO: | (mg/mL) | (mg/mL) | pH |
| Reference | 181 | 16.1 | 63.8 | 3.75 |
| Fr32-4 | 175 | 14.2 | 32.7 | 4.15 |
| Fr33-4 | 176 | 15.8 | 29.0 | 3.75 |

### Evaluation VH3 Binding Polypeptide Hexamers by Chromatography

The hexameric variant (SEQ ID NO:177) coupled resin was packed in columns and initially tested for dynamic binding capacity (DBC) for trastuzumab, trastuzumab Fab and Variable Heavy Heavy fragment (VHH). The results are summarized in Table 8.

TABLE 8
Results of dynamic binding capacity for SEQ ID NO:
177 in comparison with SEQ ID NO: 181 on 60 μm
agarose beads. Ligand density (LD) and DBC is shown.

| Multimeric | SEQ ID | LD | DBC | DBC Fab | DBC VHH |
|---|---|---|---|---|---|
| variant | NO | (mg/mL) | mAb(g/L) | (g/L) | (g/L) |
| Reference | 181 | 16.1 | 58 | 24 | 23 |
| Fr33-6 | 177 | 18.3 | 57 | 45 | 33 |

### Analysis of Alkali Clean Stability on Column

An accelerated NaOH study was made to test alkali clean stability of Fr33-6 (SEQ ID NO:177) on beads. SEQ ID NO:181 was used as reference. In this study, DBC was measured at 6 min RT followed by 4 h incubations in 0.5 M NaOH with DBC measurements between each incubation. The resin was incubated in a total of 24 h. Results from the alkali clean stability study is shown in Table 9.

TABLE 9
Summary of the DBC values for SEQ ID NO: 177
in comparison with SEQ ID NO: 181 at start
and after 4 h incubation with 0.5M NaOH

| | Reference | Reference | FR33-6 | Reference |
|---|---|---|---|---|
| Time | (g/L) | Relative (%) | (g/L) | Relative (%) |
| Start | 66 | 100 | 55 | 100 |
| 4 h | 66 | 100 | 55 | 100 |
| 8 h | 65 | 98 | 54 | 98 |
| 12 h | 65 | 98 | 54 | 98 |
| 16 h | 65 | 98 | 54 | 98 |
| 20 h | 66 | 100 | 52 | 95 |
| 24 h | 65 | 98 | 52 | 95 |

### Conclusions

Column studies show that the single A29G mutation of SEQ ID NO:175 to SEQ ID NO:176 causes the elution pH to decrease thus indicating a higher affinity. This higher affinity is also shown with Biacore in Example 3. Moreover, the increased affinity of SEQ No:177 compared to a reference leads to an increased capacity for a single 1:1 interaction such as Fab, VHH or bispecific antibody (Table 8). Surprisingly, these mutations could be made on the backbone without significantly lowering the alkali clean stability of the protein (Table 9).

### Example 5

### Selective VH Interaction Evaluation of a VH3 Binding Polypeptide on Biacore

### Summary

Antibodies are composed of the heavy (HC) and the light chain (LC) where the HC is composed of three constant (CH1-CH3) and one variable domain (VH) and the LC is composed of one constant (CL) and one variable domain (VL). Most variations in IgG1 antibody frameworks are in VH and VL. Trastuzumab and adalimumab are both IgG1 antibodies having VH class 3 and VL kappa I. Although both trastuzumab and adalimumab have VH3 frameworks, the trastuzumab and adalimumab differ in a few amino acid positions including the two positions 57 and 64 (according to the Kabat numbering system) for VH3 interaction of protein A domains (Table 10).

TABLE 10
Amino acid residues in Kabat positions as indicated of VH3 class
antibodies including trastuzumab and adalimumab are shown.
Generic and trade names of the antibodies are indicated.

| Generic name | | | | | | | | | | | | | |
|---|---|---|---|---|---|---|---|---|---|---|---|---|---|
| (Trade name) | 15 | 17 | 19 | 57 | 59 | 64 | 65 | 66 | 68 | 70 | 81 | 82a | 82b |
| Trastuzumab | G | S | R | T | Y | K | G | R | T | S | Q | N | S |
| (Herceptin) | | | | | | | | | | | | | |
| Avelumab | G | S | R | T | Y | K | G | R | T | S | Q | N | S |
| (Bavencio) | | | | | | | | | | | | | |
| Dupilumab | G | S | R | T | Y | K | G | R | T | S | Q | N | S |
| (Dupixent) | | | | | | | | | | | | | |
| Adalimumab | G | S | R | I | Y | E | G | R | T | S | Q | N | S |
| (Humira) | | | | | | | | | | | | | |
| Burosumab | G | S | K | T | N | Q | G | R | T | T | E | S | S |
| (Crysvita) | | | | | | | | | | | | | |
| Vedolizumab | G | S | K | T | Y | K | G | R | T | T | E | S | S |
| (Entyvio) | | | | | | | | | | | | | |

Even with the high similarity between trastuzumab and adalimumab, selectivity binding of VH3 binding polypeptides or multimers thereof of the present invention to the VH3 domain is different for said antibodies. Adalimumab does not bind to the VH3 binding polypeptide multimers according to the present invention. Similarly, other non-VH3 class antibodies such as burosumab (IgG1, VH1, κ|), vedolizumab (IgG1, VH1, κ∥) do not bind to the VH3 binding polypeptides of the present invention. On the other hand, antibodies that have more sequence differences than adalimumab compared to trastuzumab, but which exhibit identical amino acid sequences of the VH3 domain (for example as those described in Table 10) show binding to VH3 binding polypeptides and multimers thereof according to the present invention. Examples of such antibodies are avelumab (IgG1, VH3, λ∥) and dupilumab (IgG4, VH3, κ∥).

In the following example a representative polypeptide according to the present disclosure in a multimeric form (SEQ ID NO:177) is immobilized on a Biacore™ chip surface and is investigated for its binding selectivity to VH3 domain of mAbs from Table compared to other non-binding mAbs. It is expected that the VH3 binding polypeptide multimer (SEQ ID NO:177) does not exhibit binding affinity for adalimumab, burosumab and vedolizumab but binds trastuzumab, avelumab and dupilumab, indicating that the VH3 binding polypeptide multimer binds to the VH3 region of the mAb of trastuzumab, which VH3 region is also present in avelumab and dupilumab.

### Material and Methods

To assess binding to VH3, the VH3 binding polypeptides are immobilized on a Biacore™ CM5 chip. Different mAbs (5000 μM) are used as analyte with a flowrate of 10 μl/min for 10 minutes followed by 10 minutes dissociation. After the run, the surface is regenerated with 2 injections of 10 mM Glycine pH 1.5.

### Results

It is expected that the present data show that the VH3 binding polypeptide multimer (SEQ ID NO: 177) binds to trastuzumab, avelumab and dupilumab. However, the said VH3 binding polypeptide multimer is expected not to bind to adalimumab, burosumab or vedolizumab.

### Conclusion

Based on the expected results it is concluded that the VH3 binding polypeptides and multimers thereof according to the present invention, as exemplified by SEQ ID NO:177, specifically bind to the VH3 domain of trastuzumab. Trastuzumab and adalimumab are highly similar outside the VH3 domain and said VH3 binding polypeptides and multimers thereof do not bind to adalimumab, hence it is concluded that said VH3 binding polypeptides and multimers specifically bind to the VH3 domain of trastuzumab.

VH3 class mAbs, which are more different outside the VH3 domain compared to trastuzumab and have the same VH3 domain as trastuzumab as presented Table 10, for example avelumab and dupilumab, are expected to bind said VH3 binding polypeptide multimer. The expected results indicate that the presence of residues I and E in Kabat positions 57 and 64, respectively, within the VH3 domain adalimumab leads to the loss of its binding to the VH3 binding polypeptide multimer SEQ ID NO:177. It is concluded that the VH3 binding polypeptides according to the present invention bind to the VH3 region of trastuzumab.

### Example 6

### Specificity Testing of VH3 Binding Polypeptide Multimers Using VH1 and VH3 Class Targets

### Summary

Example 5 shows that certain mutations within the VH3 domain of trastuzumab are key for retaining trastuzumab VH3 binding. Specificity of VH3 binding polypeptide multimers can be tested by using targets with different sequences within the VH3 binding domain. Moreover, specificity of VH3 binding polypeptide multimers to VH3 class targets can be tested by investigating binding to both VH1 and VH3 class targets and to VH1/VH3 bispecific antibodies.

A set of different commercial antibodies and one bispecific antibody were used to test if the VH3 interaction of Fr33-6 (SEQ ID: 177) could be used to bind and elute antibodies on column.

The following example shows that a VH3 binding polypeptide multimer (SEQ ID NO:177) binds to VH3 class targets that have the same amino acids in positions 15, 17, 19, 57, 59, 64, 65, 66, 68, 70, 81, 82a and 82b (according to the Kabat numbering system) of the VH3 domain as that of trastuzumab. Moreover, the results show that a VH3 binding polypeptide multimer binds none of the tested VH1 containing targets but retains binding of VH1/VH3 bispecific antibodies.

### Material and Methods

Antibodies with different sequences in the VH3 binding domain (Table 11) and a set of VH1 class antibodies were tested for binding to SEQ ID NO:177 (trade name and supplier are indicated in parenthesis): Avelumab (Bavencio, Merck KGaA), Erenumab (Aimovig, Novartis), Dupilumab (Dupixent, Sanofi Genzyme), Denosumab (Xgeva, Amgen), Bevacizumab (Avastin, Roche), Adalimumab (Humira, AbbVie), Emicizumab (Hemlibra, Roche), Belimumab (Benlysta, GSK), Guselkumab (Tremfya, Jansen), Rituximab (Mabthera, Roche), Pembrolizumab (Keytruda, MSD).

TABLE 11
Amino acid residues in Kabat positions as indicated of 8 antibodies
are shown. Generic and trade names of the antibodies are indicated.

| Generic name | | | | | | | | | | | | | |
|---|---|---|---|---|---|---|---|---|---|---|---|---|---|
| (Trade name) | 15 | 17 | 19 | 57 | 59 | 64 | 65 | 66 | 68 | 70 | 81 | 82a | 82b |
| Trastuzumab | G | S | R | T | Y | K | G | R | T | S | Q | N | S |
| (Herceptin) | | | | | | | | | | | | | |
| Avelumab | G | S | R | T | Y | K | G | R | T | S | Q | N | S |
| (Bavencio) | | | | | | | | | | | | | |
| Erenumab | G | S | R | K | S | K | G | R | T | S | Q | N | S |
| (Aimovig) | | | | | | | | | | | | | |
| Dupilumab | G | S | R | T | Y | K | G | R | T | S | Q | N | S |
| (Dupixent) | | | | | | | | | | | | | |
| Denosumab | G | S | R | T | Y | K | G | R | T | S | Q | N | S |
| (Xgeva) | | | | | | | | | | | | | |
| Bevacizumab | G | S | R | P | Y | K | R | R | T | S | Q | N | S |
| (Avastin) | | | | | | | | | | | | | |
| Adalimumab | G | S | R | I | Y | E | G | R | T | S | Q | N | S |
| (Humira) | | | | | | | | | | | | | |
| Emicizumab | G | S | R | T | Y | K | G | R | T | S | Q | N | S |
| (Hemlibra) | | | | | | | | | | | | | |

Bind/elute (B/E) experiments were made in Tricorn 5/50 columns. The column was equilibrated with PBS buffer. Following, approximately 10 mg mAb diluted to 1 mg/mi in PBS was loaded to the columns via the sample pump. The column was then washed with PBS buffer at flow rate 1 mL/min. The protein was eluted with 50 mM sodium citrate pH 2.5 at flow rate 1 mL/min. The column was cleaned with 0.1 M NaOH followed by re-equilibration with PBS buffer. Binding is defined as protein comes out in the elution pool and non-binding if the protein goes in the loading flow-through (FT).

### Results

Antibodies with different sequences in the VH3 binding domain and a set of VH1 class antibodies were tested for binding to SEQ ID NO:177.

The results are summarized in Table 12. Four of the tested VH3 antibodies bound to SEQ ID NO:177, namely avelumab, erenumab, dupilumab and denosumab. VH3 class targets (avelumab, dupilumab and denosumab) that have the same amino acids in positions 15, 17, 19, 57, 59, 64, 65, 66, 68, 70, 81, 82a and 82b (according to the Kabat numbering system) as trastuzumab bound to the VH3 binding polypeptide multimer (SEQ ID NO:177). A VH3 class target, erenumab, having K in position 57 and S in position 59 bound to the VH3 binding polypeptide multimer (SEQ ID NO:177) as well. Two of the VH3 class antibodies, avastin, which has P in position 57 and R in position 65, and humira, which has I in position 57 and E in position 64 did not bind to the VH3 binding polypeptide multimer (SEQ ID NO:177).

The bispecific antibody, hemlibra, with one VH1 and one VH3 domain bound to the VH3 binding polypeptide multimer (SEQ ID NO:177).

None of the four VH1 antibodies tested bound to the VH3 binding polypeptide multimer (SEQ ID NO:177).

TABLE 12
Test binding specificity with several different
commercial antibodies (binding/no binding).

| VH class | Test substance | Binds (Y/N) |
|---|---|---|
| VH3 | Avelumab (Bavencio) | Yes |
| | Erenumab (Aimovig) | Yes |
| | Dupilumab (Dupixent) | Yes |
| | Denosumab (Xgeva) | Yes |
| | Bevacizumab (Avastin) | No |
| | Adalimumab (Humira) | No |
| VH1/VH3 | Emicizumab (Hemlibra) | Yes |
| VH1 | Belimumab (Benlysta) | No |
| | Guselkumab (Tremfya) | No |
| | Rituximab (Mabthera) | No |
| | Pembrolizumab (Keytruda) | No |

### Conclusions

A representative VH3 binding polypeptide according to the present invention was tested. The VH3 binding polypeptide multimer (SEQ ID NO:177) binds to VH3 class antibodies that have the same amino acids in positions 15, 17, 19, 57, 59, 64, 65, 66, 68, 70, 81, 82a and 82b (according to the Kabat numbering system) of the VH3 domain as trastuzumab. SEQ ID NO:177 was shown not to bind to VH1 class antibodies, while it binds to bispecific antibodies that have at least one VH3 domain. It is concluded that the VH3 binding polypeptides according to the present invention bind to the VH3 region which is present in amongst others in trastuzumab.

It is further concluded that the VH3 interaction of VH3 binding polypeptides or multimers thereof according to the present invention, as exemplified here by SEQ ID NO:177, can be used for purification of antibodies even though Fc interaction is abolished. This interaction works for some VH3 class antibodies but not for all. Moreover, none of VH1 class antibodies bound to the resin comprising SEQ NO:177. Interestingly, a bispecific antibody with one binding arm could be purified in B/E mode indicating that only one interaction is sufficient to use as a purification tool.

### Example 7

### Separation of VH1/VH3 Bispecific Antibodies from VH3/VH3 Homodimers Using a VH3 Binding Polypeptide Multimer

### Summary

This Example describes the separation of VH1/VH1 and VH3/VH3 homodimers from an asymmetric bispecific antibody heterodimer consisting of VH1/VH3 class pairing heavy chains. In production of an asymmetric VH1/VH3 bispecific antibody the two heavy chains must pair correctly. Mis-paired heavy chains are VH1/VH1 or VH3/VH3 homodimers.

As shown in Example 6 and in Table 12, VH1/VH1 homodimers do not exhibit binding to SEQ ID NO:177 and therefore pass through a resin coupled to the alkali clean stabile VH3 binding polypeptide hexamers (SEQ ID NO:177) without binding. In the following example it is proven that VH3/VH3 homodimers exhibit stronger binding to a resin coupled to alkali clean stabile VH3 binding polypeptide hexamers (SEQ ID NO:177). Without being bound by theory, it is expected that this is due to the avidity effect created by the presence of two VH3 targets in the homodimers. Thus VH3/VH3 homodimers may be eluted at a lower pH in a gradient than an asymmetric VH1/VH3 bispecific antibody.

It is concluded that a resin coupled to alkali clean stabile VH3 binding polypeptides or multimers thereof, such as the hexamer SEQ ID NO:177, has the advantageous property that it enables the simultaneous chromatographic separation of VH1/VH1 and VH3/VH3 homodimers from VH1/VH3 heterodimers. By utilizing this effect of said resin it is possible to separate mis-paired homodimers from correctly paired heterodimers. Thus, resin comprising an alkali clean stabile VH3 binding polypeptide or multimer thereof according to the present invention enables purification of an asymmetric VH1/VH3 bispecific antibody or fragment thereof in a superior way using a single chromatographic step.

### Separation of Homodimers and Heterodimers in an Emicizumab (Hemlibra®) Feed.

### Materials and Methods

Emicizumab was ordered from Thermo Fisher (GeneArt, Regensburg, Germany) according to published sequence (KEGG drug database). The bispecific antibody was produced in Expi293 cells and delivered in sterile filtered cell culture medium 10× concentrated. The target protein concentration was approximately 1 g/L as measured from SDS-PAGE. The cell culture medium was loaded onto either a control column containing resin that is coupled to SEQ ID NO:181 (as Fc and VH3 binding reference) or a column containing resin that is coupled to SEQ ID NO:177 that only binds through VH3 interaction according to the present invention. Protein of SEQ ID NO:177 was coupled to two agarose base matrixes of 60 and 50 μm to see if bead size could affect resolution. Approximately 5 mL feed was loaded onto a 1 mL column packed in Tricorn 5/50 and eluted in a gradient from pH 6.5 to 2.5 using the following buffers: equilibration buffer, 20 mM sodium phosphate in 150 mM sodium chloride, pH 7.2; gradient from 50 mM sodium citrate pH 6.5 to 50 mM sodium citrate pH 2.5, followed by cleaning with 0.5 M NaOH. When the cycle was finished, the column was regenerated with equilibration buffer again. In order to separate between homodimers and heterodimers, LC/MS was used to measure the mass of the protein in each elution fraction.

### Results

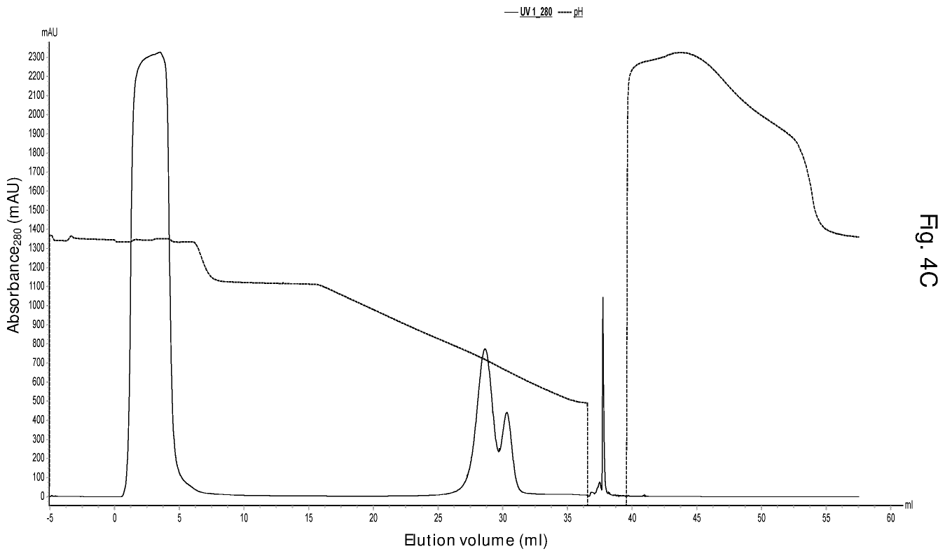

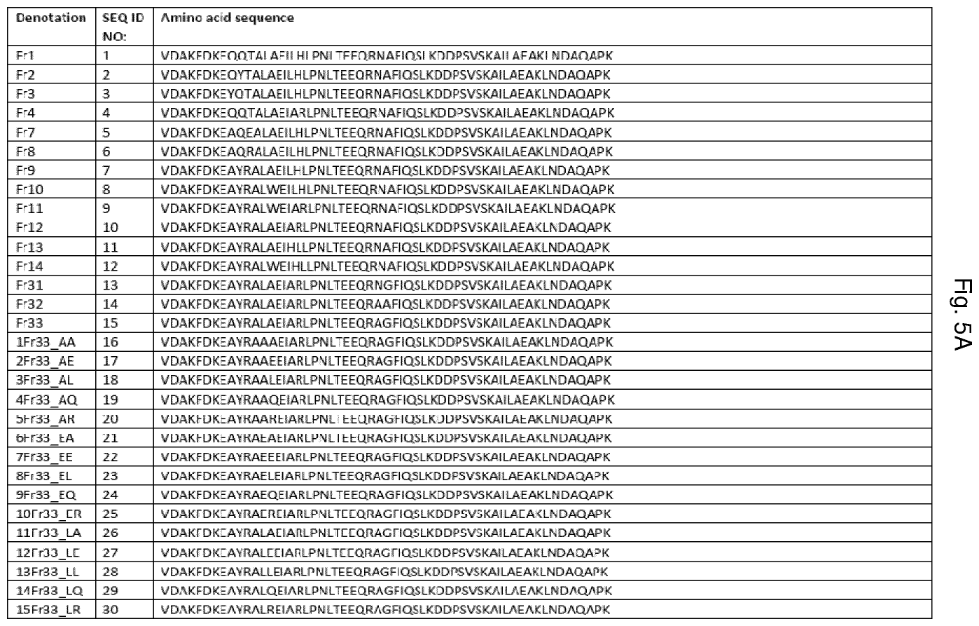

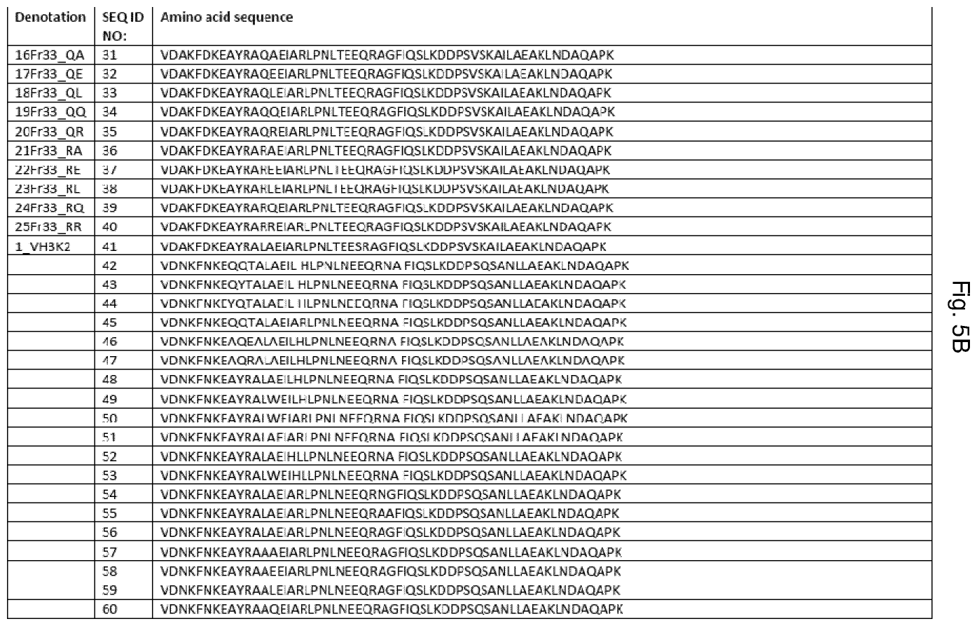

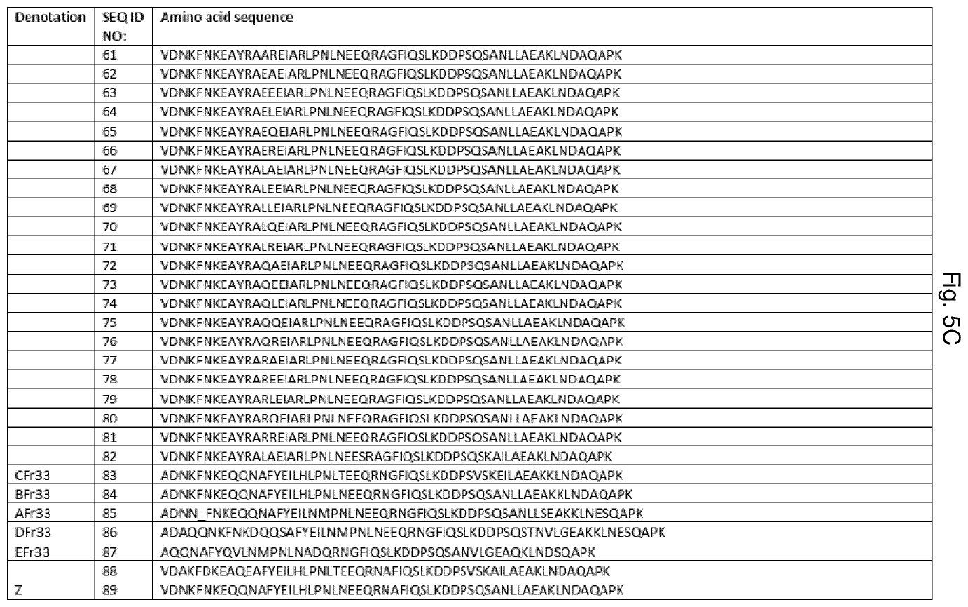

Emicizumab feed comprises VH1/VH3 bispecific antibodies and also VH3/VH3 mis-paired homodimers. Using a control column containing resin that is coupled to SEQ ID NO:181 that is hexamers of an alkali clean stabile polypeptide that binds both Fc and Fab fragments of trastuzumab (SEQ ID NO:88), only one elution peak can be detected (FIG. 4A). Thus, in a control column containing resin that is coupled to SEQ ID NO:181, VH1/VH3 bispecific antibodies and VH3/VH3 mis-paired homodimers in emicizumab feed cannot be separated from each other. On the contrary, using a column containing resin according to the present invention that is coupled to SEQ ID NO:177 that is hexamers of an alkali clean stabile VH3 binding polypeptide that does not bind to the Fc fragment of trastuzumab (SEQ ID NO:15), two elution peaks can be detected (FIGS. 4B and 4C). Thus, in a column containing resin according to the present invention that is coupled to SEQ ID NO:177, VH1/VH3 bispecific antibodies and VH3/VH3 mis-paired homodimers in emicizumab feed can readily be separated from each other. The resolution was best for the smaller 50 μm bead size (FIG. 4C). Eluate fractions from the 60 μm bead was analyzed with LC/MS. Homodimer is theoretically 485 Da larger than the heterodimer based on the amino-acid sequence and could be detected in fraction 7 and 8 (Table 13). The results indicate that the homodimer elutes at a slightly lower pH due to the double interaction.

TABLE 13
LC/MS analysis of eluate fractions from
SEQ ID NO: 177 on 60 μm agarose bead.

| | Heterodimer | Homodimer | |
|---|---|---|---|
| Fraction | (VH1/VH3) | (VH3/VH3) | Delta mass |
| A1 | 148495 Da | | |
| A2 | 148495 Da | | |
| A3 | 148495 Da | | |
| A4 | 148495 Da | | |
| A5 | 148495 Da | | |
| A6 | 148495 Da | 148980 Da | Both |
| A7 | | 148980 Da | +485 Da |
| A8 | | 148980 Da | +485 Da |

Mismatched VH3/VH3 homodimers bind stronger to SEQ ID NO:177 resin in comparison to the VH1/VH3 heterodimer of bispecific antibodies. This is expected to be due to the presence of multiple binding sites and avidity effect. Therefore the VH3/VH3 homodimers are eluted at a lower pH in a column containing a resin that is coupled to SEQ ID NO:177 according to the present invention. Thus, in the chromatogram in FIGS. 4B and 4C there is a distinct peak eluting at pH-2.9 corresponding to the elution of VH3/VH3 homodimers and a distinct peak at a higher pH corresponding to the elution of VH1/VH3 bispecific antibodies.

### Conclusions

It is concluded that separation of mis-paired homodimer against heterodimer could be achieved with any VH3 binding polypeptide or multimer thereof according to the present invention as shown here for Fr33-6 (SEQ ID No:177). This separation is based on binding to the antibody VH3 interaction. When the antibody is mis-paired and thus has two VH3 domain which may interact with the polypeptides of the present invention, it binds harder to the column and elutes at a slightly more acidic pH. This separation could be performed due to the removal of the Fc interaction of protein A.

It is concluded that a resin composed of an alkali clean stabile VH3 binding polypeptide or multimer thereof, such as the hexamer SEQ ID NO:177, has the advantageous property that it enables the chromatographic separation of mis-paired VH1/VH1 and VH3/VH3 homodimers from VH1/VH3 bispecific antibodies. Separation of VH1/VH1 and VH3/VH3 homodimers from VH1/VH3 bispecific antibodies is made possible by the advantageous characteristics of an alkali clean stabile VH3 binding polypeptide or multimers thereof. As shown herein the alkali clean stabile VH3 binding polypeptide hexamer SEQ ID NO:177, as well as the alkali clean stabile VH3 binding polypeptide SEQ ID NO:15 that does not bind to the Fc fragment of trastuzumab, do not bind to an Fc fragment and a VH1 domain of an antibody but exhibit high affinity binding to the VH3 domain of an antibody. The alkali clean stabile properties of said polypeptides and multimers thereof, enable the use of said VH3 binding polypeptides and/or multimers in chromatographic separation of VH1/VH1 and VH3/VH3 homodimers from VH1/VH3 bispecific antibodies. Additionally, said properties allow for repeated use of a resin comprising said polypeptides and/or multimers with intermittent cleaning steps while significantly retaining binding properties thereof.

By utilizing this superior effect of a resin composed of an alkali clean stabile VH3 binding polypeptides or multimers thereof according to the present invention, it is possible to revolutionize the production of bispecific antibodies, make it more affordable to create bispecific antibodies and therefore, facilitate the production of therapeutic drugs comprising bispecific antibodies. Purification of properly paired VH1/VH3 bispecific antibodies simultaneously from VH1/VH1 and VH3/VH3 homodimers is advantageous for the production of VH1/VH3 bispecific antibody therapeutics with high purity.

### Example 8

### Evaluation of VH3 Binding Polypeptides in a Different Scaffold

In order to evaluate the effect of mutations in positions 9, 10, 11, 13, 14, 17, 18, 28 and 29 in a different scaffold, SEQ ID NO:89 is modified with the amino acid residues according to Tables 1, 3 and 5 in the nine positions. The resulting VH3 binding polypeptides are listed as SEQ ID NO:42-56.

VH3 binding polypeptides (SEQ ID NO:42-45) are generated essentially as described in the Example 1 section entitled “Generation of His-purified candidate polypeptides”. Evaluation and analysis is performed essentially as described in Example 1 sections entitled “Biacore analysis of binding affinity for Fc and Fab fragments of trastuzumab” and “Biacore analysis of alkali clean stability”.

VH3 binding polypeptides (SEQ ID NO:42-45) are evaluated together with a control that binds both trastuzumab Fc and Fab fragments (SEQ ID NO:89), a non-Fc-binding control (SEQ ID NO:180) and a non-Fc binding control that is not alkali clean stabile (Negative CTRL).

The following aspects are evaluated:
(1) Affinity assessment of Fc interaction (tested through high concentration injection of Fc fragment generated from trastuzumab).
(2) Affinity assessment of Fab interaction (tested through high concentration injection of Fab fragment generated from trastuzumab).
(3) Alkali clean stability (tested by reduction of trastuzumab binding after increasing number of treatments with 0.5 M NaOH).

VH3 binding polypeptides (SEQ ID NO:46-53) are generated essentially as described in the Example 2 section entitled “Generation of His-purified candidate polypeptides”. Evaluation and analysis is performed essentially as described in Example 2 sections entitled “Biacore analysis of binding affinity for Fc fragment of trastuzumab and trastuzumab” and “Biacore analysis of alkali clean stability”.

VH3 binding polypeptides (SEQ ID NO:46-53) are evaluated together with a control that binds both trastuzumab Fc and Fab fragments (SEQ ID NO:89) and a non-Fc binding control that is not alkali clean stabile (Negative CTRL).

The following aspects are evaluated:
(1) Affinity assessment of Fc interaction (tested through high concentration injection of Fc fragment generated from trastuzumab).
(2) Affinity assessment of trastuzumab interaction (tested through high concentration injection of trastuzumab).
(3) Alkali clean stability (tested by reduction of trastuzumab binding after increasing number of treatments with 0.5 M NaOH).

VH3 binding polypeptides (SEQ ID NO:54-56) are generated essentially as described in the Example 3 section entitled “Generation of His-purified candidate polypeptides”. Evaluation and analysis is performed essentially as described in Example 3 sections entitled “Biacore analysis of binding affinity for Fc and Fab fragments of trastuzumab and trastuzumab” and “Biacore analysis of alkali clean stability”.

VH3 binding polypeptides (SEQ ID NO:54-56) are evaluated together with a control that binds both trastuzumab Fc and Fab fragments (SEQ ID NO:89) and a non-Fc binding control that is not alkali clean stabile (Negative CTRL).

The following aspects are evaluated:
(1) Affinity assessment of Fc interaction (tested through high concentration injection of Fc fragment generated from trastuzumab).
(2) Affinity assessment of Fab interaction (tested through high concentration injection of Fab fragment generated from trastuzumab).
(3) Affinity assessment of trastuzumab interaction (tested through high concentration injection of trastuzumab).
(4) Alkali clean stability (tested by reduction of trastuzumab binding after increasing number of treatments with 0.5 M NaOH).

It is expected that the VH3 binding polypeptides (SEQ ID NO: 42-56) are alkali clean stabile or exhibit improved alkali clean stability properties, exhibit VH3 binding affinity and exhibit significantly reduced or abolished affinity for the Fc region of trastuzumab compared to SEQ ID NO:89.

### Example 9

### Evaluation of Additional VH3 Binding Polypeptides in Additional Scaffolds

In order to evaluate the effect of amino acids “AYRLAARAG” in positions X₉X₁₀X₁₁X₁₃X₁₄X₁₇X₁₈X₂₈X₂₉ in additional scaffolds, SpA protein native domains A (SEQ ID NO: 92), B (SEQ ID NO: 91), C (SEQ ID NO: 90), D (SEQ ID NO: 93) and E (SEQ ID NO: 94) are modified with amino acid residues to have the same amino acids in positions 9, 10, 11, 13, 14, 17, 18, 28 and 29 as set out in Table 5 for the VH3 binding polypeptide Fr33 (SEQ ID NO:15). The resulting VH3 binding polypeptide on SEQ ID NO:92 background (AFr33) is listed as SEQ ID NO:85, the resulting VH3 binding polypeptide on SEQ ID NO:91 background (BFr33) is listed as SEQ ID NO:84, the resulting VH3 binding polypeptide on SEQ ID NO:90 background (CFr33) is listed as SEQ ID NO:83, the resulting VH3 binding polypeptide on SEQ ID NO:93 background (DFr33) is listed as SEQ ID NO:86 and the resulting VH3 binding polypeptide on SEQ ID NO:94 background (EFr33) is listed as SEQ ID NO:87.

VH3 binding polypeptides (SEQ ID NO:83-87) are generated essentially as described in the Example 3 section entitled “Generation of His-purified candidate polypeptides”. Evaluation and analysis is performed essentially as described in Example 3 sections entitled “Biacore analysis of binding affinity for Fc and Fab fragments of trastuzumab and trastuzumab” and “Biacore analysis of alkali clean stability”.

VH3 binding polypeptides (SEQ ID NO:83-87) are evaluated pairwise with their corresponding scaffold polypeptide (SEQ ID NO:90-94) that binds both trastuzumab Fc and Fab fragments as reference, and together with a non-Fc binding control that is not alkali clean stabile (Negative CTRL).

The following aspects are evaluated:
(1) Affinity assessment of Fc interaction (tested through high concentration injection of Fc fragment generated from trastuzumab).
(2) Affinity assessment of Fab interaction (tested through high concentration injection of Fab fragment generated from trastuzumab).
(3) Affinity assessment of trastuzumab interaction (tested through high concentration injection of trastuzumab).
(4) Alkali clean stability (tested by reduction of trastuzumab binding after increasing number of treatments with 0.5 M NaOH).

It is expected that the VH3 binding polypeptides (SEQ ID NO: 83-87) are alkali clean stabile or exhibit improved alkali clean stability properties, exhibit VH3 binding affinity and exhibit significantly reduced or abolished affinity for the Fc region of trastuzumab compared to their corresponding scaffold polypeptide (SEQ ID NO:90-94).

### Example 10

### Evaluation of Additional VH3 Binding Polypeptides in Two Different Scaffolds

In order to evaluate the effect of mutations in positions 13 and 14 in two different scaffolds, alkali clean stabile VH3 binding polypeptides (Fr33) SEQ ID NO:15 and SEQ ID NO:56 are modified with the amino acid residues according to Table 14 in the two positions. The resulting VH3 binding polypeptides on SEQ ID NO:15 background are listed as SEQ ID NO:16-40 and the resulting VH3 binding polypeptides on SEQ ID NO:56 background are listed as SEQ ID NO:57-81. Moreover, the effect of a glutamine (Q) to serine (S) mutation in position 26 is evaluated in the two different scaffolds, alkali clean stabile VH3 binding polypeptides (Fr33) SEQ ID NO:15 and SEQ ID NO:56. The resulting VH3 binding polypeptide (VH3K2) on SEQ ID NO:15 background is listed as SEQ ID NO:41 and the resulting VH3 binding polypeptide (VH3K2) on SEQ ID NO:56 background is listed as SEQ ID NO:82.

TABLE 14
Summary of mutations (SEQ ID NO: 16-40 and SEQ ID NO: 57-81)

| | SEQ ID NO | | | | | | | | | | |
|---|---|---|---|---|---|---|---|---|---|---|---|
| VH3 binding | Scaffold: | Scaffold: | Position | | | | | | | | |
| polypeptide | 15 | 56 | 9 | 10 | 11 | 13 | 14 | 17 | 18 | 28 | 29 |
| 1Fr33_AA | 16 | 57 | A | Y | R | A | A | A | R | A | G |
| 2Fr33_AE | 17 | 58 | A | Y | R | A | E | A | R | A | G |
| 3Fr33_AL | 18 | 59 | A | Y | R | A | L | A | R | A | G |
| 4Fr33_AQ | 19 | 60 | A | Y | R | A | Q | A | R | A | G |
| 5Fr33_AR | 20 | 61 | A | Y | R | A | R | A | R | A | G |
| 6Fr33_EA | 21 | 62 | A | Y | R | E | A | A | R | A | G |
| 7Fr33_EE | 22 | 63 | A | Y | R | E | E | A | R | A | G |
| 8Fr33_EL | 23 | 64 | A | Y | R | E | L | A | R | A | G |
| 9Fr33_EQ | 24 | 65 | A | Y | R | E | Q | A | R | A | G |
| 10Fr33_ER | 25 | 66 | A | Y | R | E | R | A | R | A | G |
| 11Fr33_LA | 26 | 67 | A | Y | R | L | A | A | R | A | G |
| 12Fr33_LE | 27 | 68 | A | Y | R | L | E | A | R | A | G |
| 13Fr33_LL | 28 | 69 | A | Y | R | L | L | A | R | A | G |
| 14Fr33_LQ | 29 | 70 | A | Y | R | L | Q | A | R | A | G |
| 15Fr33_LR | 30 | 71 | A | Y | R | L | R | A | R | A | G |
| 16Fr33_QA | 31 | 72 | A | Y | R | Q | A | A | R | A | G |
| 17Fr33_QE | 32 | 73 | A | Y | R | Q | E | A | R | A | G |
| 18Fr33_QL | 33 | 74 | A | Y | R | Q | L | A | R | A | G |
| 19Fr33_QQ | 34 | 75 | A | Y | R | Q | Q | A | R | A | G |
| 20Fr33_QR | 35 | 76 | A | Y | R | Q | R | A | R | A | G |
| 21Fr33_RA | 36 | 77 | A | Y | R | R | A | A | R | A | G |
| 22Fr33_RE | 37 | 78 | A | Y | R | R | E | A | R | A | G |
| 23Fr33_RL | 38 | 79 | A | Y | R | R | L | A | R | A | G |
| 24Fr33_RQ | 39 | 80 | A | Y | R | R | Q | A | R | A | G |
| 25Fr33_RR | 40 | 81 | A | Y | R | R | R | A | R | A | G |

VH3 binding polypeptides (SEQ ID NO:16-41 and SEQ ID NO:57-82) are generated essentially as described in the Example 3 section entitled “Generation of His-purified candidate polypeptides”. Evaluation and analysis is performed essentially as described in Example 3 sections entitled “Biacore analysis of binding affinity for Fc and Fab fragments of trastuzumab and trastuzumab” and “Biacore analysis of alkali clean stability”.

VH3 binding polypeptides (SEQ ID NO:16-41) are evaluated together with a control that binds both trastuzumab Fc and Fab fragments (SEQ ID NO:88) and a non-Fc binding control that is not alkali clean stabile (Negative CTRL).

VH3 binding polypeptides (SEQ ID NO:57-82) are evaluated together with a control that binds both trastuzumab Fc and Fab fragments (SEQ ID NO:89) and a non-Fc binding control that is not alkali clean stabile (Negative CTRL).

The following aspects are evaluated:
(1) Affinity assessment of Fc interaction (tested through high concentration injection of Fc fragment generated from trastuzumab).
(2) Affinity assessment of Fab interaction (tested through high concentration injection of Fab fragment generated from trastuzumab).
(3) Affinity assessment of trastuzumab interaction (tested through high concentration injection of trastuzumab).
(4) Alkali clean stability (tested by reduction of trastuzumab binding after increasing number of treatments with 0.5 M NaOH).

It is expected that the VH3 binding polypeptides (SEQ ID NO:16-41) are alkali clean stabile or exhibit improved alkali clean stability properties, exhibit VH3 binding affinity and exhibit significantly reduced or abolished affinity for the Fc region of trastuzumab compared to SEQ ID NO:88.

It is expected that the VH3 binding polypeptides (SEQ ID NO:57-82) are alkali clean stabile or exhibit improved alkali clean stability properties, exhibit VH3 binding affinity and exhibit significantly reduced or abolished affinity for the Fc region of trastuzumab compared to SEQ ID NO:89.

### Example 11

### Biacore Evaluation of IgG Binding Polypeptides after Knockout Mutations for Fc Interaction

### Biacore Analysis of Binding Affinity for Fc and Fab Fragments of Trastuzumab

To assess binding to Fc (SEQ ID NO:184) and VH3/Fab (SEQ ID NO:182 and 183), IgG binding polypeptides before mutations (group 1; G1) and the polypeptides after knockout mutations of Fc interaction (group 2-9) were immobilized on a Biacore CM5 chip and thereafter Trastuzumab Fc (500 nM) and trastuzumab Fab (500 nM) fragments were used as analyte.

Each Fc knock out group have the mutations according to the following (at positions corresponding to the below positions in SEQ ID NO:89):
Group 2 (G2): 9A, 10Y, 11R, 13L, 14A, 17A, 18R, 28A, 29G
Group 3 (G3): 9A, 10Y, 11R, 13L, 14A, 17A, 18R, 26S, 28A, 29G
Group 4 (G4): 9A, 10Y, 11E, 13R, 14A, 17A, 18R, 28A, 29G
Group 5 (G5): 9A, 10Y, 11E, 13R, 14A, 17A, 18L, 28A, 29G
Group 6 (G6): 9A, 10Q, 11R, 13R, 14A, 17L, 18R, 28A, 29G
Group 7 (G7): 9A, 10Y, 11E, 13L, 14R, 17A, 18R, 28A, 29G
Group 8 (G8): 9A, 10Y, 11E, 13A, 14R, 17A, 18R, 28A, 29G
Group 9 (G9): 9Y, 10Q, 11R, 13L, 14A, 17A, 18L, 28A, 29G

The sequences comprised in each group are explained in Table 15 below, referring to the SEQ ID NO (SID).

TABLE 15
overview of Fc knock of IgG polypeptides (SID = SEQ ID NO)

| Group | G1 | G2 | G3 | G4 | G5 | G6 | G7 | G8 | G9 |
|---|---|---|---|---|---|---|---|---|---|
| SID | 94 | 194 | — | 222 | 225 | 228 | 231 | 234 | 237 |
| SID | 91 | 197 | 210 | 223 | 226 | 229 | 232 | 235 | 238 |
| SID | 90 | 198 | 211 | 224 | 227 | 230 | 233 | 236 | 239 |
| SID | 93 | 195 | — | — | — | — | — | — | — |
| SID | 92 | 196 | 209 | — | — | — | — | — | — |
| SID | 89 | 199 | 212 | — | — | — | — | — | — |
| SID | 185 | 200 | 213 | — | — | — | — | — | — |
| SID | 186 | 201 | 214 | — | — | — | — | — | — |
| SID | 187 | 202 | 215 | — | — | — | — | — | — |
| SID | 188 | 203 | 216 | — | — | — | — | — | — |
| SID | 189 | 204 | 217 | — | — | — | — | — | — |
| SID | 190 | 205 | 218 | — | — | — | — | — | — |
| SID | 191 | 206 | 219 | — | — | — | — | — | — |
| SID | 192 | 207 | 220 | — | — | — | — | — | — |
| SID | 193 | 208 | 221 | — | — | — | — | — | — |

Hence, each row in Table 15 refers to the same base sequence, which is the sequence indicated in the column marked G1. Following columns indicates which sequences belong to each group for that particular base sequence.

The constructs were expressed and purified as disclosed above.

### Biacore Binding Analysis:

Materials and equipment used were as follows: CM5 sensor chips (Cytiva™) Biacore™ NHS coupling kit (Cytiva™), the polypeptides (SEQ ID NO:89-SEQ ID NO: 94 and SEQ ID NO:185-SEQ ID NO:239); Biacore™ 8K+(Cytiva™); Fab-fragment and Fc-fragment (prepared from trastuzumab in-house as disclosed above).

Immobilization was performed using a standard method in Biacore software with coupling of polypeptide variants in Flow Cell 2 (FC2) and activation/inactivation in Flow cell 1 (FC1). The polypeptide variants used for immobilization were diluted in acetate-buffer (Cytiva™) with a pH value ≥1 unit below the pI of the polypeptide at a concentration ranging from 10-50 μg/ml. The immobilization levels did vary somewhat between different polypeptide variants (approx. 700-2000 Ru, see Table 16).

In each run the polypeptide was immobilized in FC2. Multiple chips were used until all candidates were tested.

Biacore™ method for binding analysis:
Running buffer: PBS-P+
Flow rate: 10 μl/min
Sample injection: 600 s/10 min over both Flow Cells (FC1 and FC2)
Dissociation time: 600 s/10 min
Regeneration: 10 mM Glycin-HCl pH 1.5, 30 μl/min, 2×30 s

Injections of analytes Fab and Fc fragment of trastuzumab at 500 nM concentration in running buffer.

All sensorgrams were generated as reference subtracted and the responses as the difference between baseline before injection and signal just before end of injection.

### Results

### Biacore Analysis of Binding for Fc and Fab Fragments of Trastuzumab

The response levels of the trastuzumab Fc and Fab interactions of the polypeptide variants are summarized in Table 16 (levels rounded to the nearest multiple of 50).

TABLE 16
Results for Fc binding and Fab binding

| | | Immobilization | Fc | Fab |
|---|---|---|---|---|
| Group | SEQ ID NO | level | binding | binding |
| Group 1 | SEQ ID NO: 94 | 783 | 1650 | 250 |
| | SEQ ID NO: 93 | 861 | 1850 | 200 |
| | SEQ ID NO: 92 | 802 | 1700 | 500 |
| | SEQ ID NO: 91 | 986 | 2200 | 750 |
| | SEQ ID NO: 90 | 1098 | 2100 | 2300 |
| | SEQ ID NO: 89 | 892 | 1750 | 0 |
| | SEQ ID NO: 185 | 711 | 1500 | 0 |
| | SEQ ID NO: 186 | 953 | 2100 | 0 |
| | SEQ ID NO: 187 | 1151 | 2200 | 0 |
| | SEQ ID NO: 188 | 1321 | 2850 | 0 |
| | SEQ ID NO: 189 | 1375 | 3250 | 0 |
| | SEQ ID NO: 190 | 1237 | 2850 | 0 |
| | SEQ ID NO: 191 | 1432 | 2800 | 550 |
| | SEQ ID NO: 192 | 869 | 1800 | 0 |
| | SEQ ID NO: 193 | 2081 | 4750 | 0 |
| Group 2 | SEQ ID NO: 194 | 1257 | 50 | 250 |
| | SEQ ID NO: 195 | 1360 | 0 | 100 |
| | SEQ ID NO: 196 | 1342 | 0 | 2400 |
| | SEQ ID NO: 197 | 1363 | 0 | 550 |
| | SEQ ID NO: 198 | 1333 | 0 | 600 |
| | SEQ ID NO: 199 | 1145 | 0 | 550 |
| | SEQ ID NO: 200 | 1372 | 0 | 150 |
| | SEQ ID NO: 201 | 1366 | 0 | 2550 |
| | SEQ ID NO: 202 | 1354 | 0 | 3050 |
| | SEQ ID NO: 203 | 1383 | 0 | 0 |
| | SEQ ID NO: 204 | 1449 | 0 | 0 |
| | SEQ ID NO: 205 | 1144 | 0 | 0 |
| | SEQ ID NO: 206 | 1461 | 0 | 2500 |
| | SEQ ID NO: 207 | 1090 | 0 | 150 |
| | SEQ ID NO: 208 | 1987 | 0 | 0 |
| Group 3 | SEQ ID NO: 209 | 1174 | 0 | 100 |
| | SEQ ID NO: 210 | 1213 | 0 | 150 |
| | SEQ ID NO: 211 | 1226 | 0 | 2250 |
| | SEQ ID NO: 212 | 1244 | 0 | 150 |
| | SEQ ID NO: 213 | 1158 | 0 | 250 |
| | SEQ ID NO: 214 | 1243 | 0 | 2650 |
| | SEQ ID NO: 215 | 1231 | 0 | 3550 |
| | SEQ ID NO: 216 | 1377 | 0 | 0 |
| | SEQ ID NO: 217 | 1505 | 0 | 0 |
| | SEQ ID NO: 218 | 1592 | 0 | 0 |
| | SEQ ID NO: 219 | 1698 | 0 | 3100 |
| | SEQ ID NO: 220 | 1293 | 0 | 200 |
| | SEQ ID NO: 221 | 1343 | 0 | 50 |
| Group 4 | SEQ ID NO: 222 | 1223 | 0 | 350 |
| | SEQ ID NO: 223 | 1227 | 0 | 1350 |
| | SEQ ID NO: 224 | 1311 | 0 | 3100 |
| Group 5 | SEQ ID NO: 225 | 1210 | 50 | 350 |
| | SEQ ID NO: 226 | 1058 | 0 | 1250 |
| | SEQ ID NO: 227 | 1210 | 0 | 2600 |
| Group 6 | SEQ ID NO: 228 | 1179 | 0 | 300 |
| | SEQ ID NO: 229 | 1401 | 0 | 1600 |
| | SEQ ID NO: 230 | 1424 | 0 | 3400 |
| Group 7 | SEQ ID NO: 231 | 1971 | 0 | 650 |
| | SEQ ID NO: 232 | 1227 | 0 | 500 |
| | SEQ ID NO: 233 | 1333 | 0 | 2150 |
| Group 8 | SEQ ID NO: 234 | 1866 | 0 | 500 |
| | SEQ ID NO: 235 | 1421 | 0 | 1200 |
| | SEQ ID NO: 236 | 1361 | 0 | 2650 |
| Group 9 | SEQ ID NO: 237 | 1096 | 0 | 150 |
| | SEQ ID NO: 238 | 1057 | 0 | 850 |
| | SEQ ID NO: 239 | 1111 | 0 | 2200 |

In group 1, polypeptide sequences SEQ ID NO 90-SEQ ID NO:94) (non-mutated wt domains) and SEQ ID NO:191 exhibit binding to Fc fragment as well as the Fab (VH-3) of trastuzumab. Polypeptide sequences SEQ ID NO:89, SEQ ID NO:185-SEQ ID NO:190 and SEQ ID NO:192-SEQ ID NO:193 exhibit no binding to the Fab (VH-3) fragment of trastuzumab.

The data shows that the IgG binding polypeptide variants that were subjected to Fc knockout mutations according to group 2 (SEQ ID NO:194-208) have an abolished or significantly reduced binding affinity for the Fc fragment of trastuzumab compared to their corresponding sequence among the original/parent polypeptides (group 1).

Different mutations to knock out Fc interaction can affect the VH3 interaction (Fab-interaction) differently on different IgG polypeptides, in terms of a stronger or weaker VH3/Fab interaction, see table 16.

Interestingly, it can be observed that in some cases, such as SEQ ID NO:89, SEQ ID NO:185, SEQ ID NO:186, SEQ ID NO:187 and SEQ ID NO:192, the mutations in both of the groups 2 and 3 (see SEQ ID NO:199-SEQ ID NO:202 and SEQ ID NO:207, SEQ ID NO:212-SEQ ID NO:215 and SEQ ID NO:220) provided a Fab binding activity that was not present for the same domains in group 1, while the Fc binding activity is abolished.

SEQ ID NO:188-SEQ ID NO:190 did not have a Fab binding in Group 1, and the mutations according to the present disclosure did not provide a Fab binding. However, the Fc binding was efficiently knocked-out by the mutations in the groups tested (Groups 2 and 3).

Thus, the mutations according to the present disclosure have been shown to enhance or retain already existing Fab binding activity, while also knocking out the previously existing Fc binding activity, in a variety of Protein A domains or Protein A domain variants. In some instances, a Fab binding activity may further be introduced when previously non-existing for the domain, although this was not the case for all domains that initially lacked the Fab binding activity. However, all domains that initially had a Fab/VH3 binding activity, showed a retained or enhanced Fab/VH3 binding activity, with a substantially abolished Fc binding activity.

Hence, polypeptides according to the scope of the claims have been shown to be VH3 binding polypeptides with weak or no Fc binding activity.

### ITEMIZED LIST OF EMBODIMENTS

1. VH3 binding polypeptide derived from a Staphylococcus Protein A (SpA) or any domain thereof, wherein said polypeptide has binding affinity for a VH3 region of trastuzumab, and has lower binding affinity for an Fc region of trastuzumab, compared to the binding affinity of SEQ ID NO:88 or of SEQ ID NO:89 for the same Fc region and wherein said VH3 binding polypeptide is alkali clean stabile.
2. VH3 binding polypeptide comprising a sequence A, which Sequence A consists of an amino acid sequence selected from i), ii) and iii), wherein i), ii) and iii) are defined as follows:

i)
(SEQ ID NO: 95)
X₈X₉X₁₀X₁₁AX₁₃X₁₄X₁₅X₁₆X₁₇X₁₈X₁₉PNLX₂₃X₂₄X₂₅X₂₆RX₂₈X₂₉FIQSL
X₃₅X₃₆

wherein, independently from each other,
X₈ is selected from E, D and A;
X₁₅ is selected from E and Q;
X₁₆ is selected from I and V;
X₁₉ is selected from L and M;
X₂₃ is selected from N and T;
X₂₄ is selected from A and E, such as is E;
X₂₅ is selected from D and E, such as is E;
X₃₅ is selected from K, R and H;
X₃₆ is selected from D and H;
ii) an amino acid sequence which has at least 83% identity to a sequence defined by i)
iii) an amino acid sequence which has at least 70% identity to any sequence selected from the group consisting of: residues 8-36 in SEQ ID NO:88, residues 8-36 in SEQ ID NO:89, residues 8-36 in SEQ ID NO:90, residues 8-36 in SEQ ID NO:91, residues 8-36 in SEQ ID NO:92, residues 11-39 in SEQ ID NO:93 and residues 1-29 in SEQ ID NO:94,
   wherein additionally, in each of i), ii) and iii) independently from each other,
X₉ is selected from Q, Y and A;
X₁₀ is selected from Q and Y;
X₁₁ is selected from T, E and R;
X₁₃ is selected from L, E, R, A and Q;
X₁₄ is selected from L, E, R, A, Q and W;
X₁₇ is selected from A, H and L;
X₁₈ is selected from R, L and H;
X₂₆ is selected from Q and S;
X₂₈ is selected from N and A; and
X₂₉ is selected from A and G.
3. VH3 binding polypeptide according to item 2, wherein said VH3 binding polypeptide has binding affinity for a VH3 region of trastuzumab and has a lower binding affinity for an Fc region of trastuzumab than SEQ ID NO:88 or SEQ ID NO:89.
4. VH3 binding polypeptide according to any one of items 1-3, wherein said trastuzumab is defined the heavy chain amino acid sequence according to SEQ ID NO:183 and the light chain amino acid sequence according to SEQ ID NO:184.
5. VH3 binding polypeptide according to any one of items 2-4, wherein X₉ is selected from Q and Y.
6. VH3 binding polypeptide according to any one of items 2-4, wherein X₉ is selected from Q and A.
7. VH3 binding polypeptide according to any one of items 2-4, wherein X₉ is selected from A and Y.
8. VH3 binding polypeptide according to any one of items 2-4, 6 and 7, wherein X₉ is A.
9. VH3 binding polypeptide according to any one of items 2-4, 5 and 7, wherein X₉ is Y.
10. VH3 binding polypeptide according to any one of items 2-6, wherein X₉ is Q.
11. VH3 binding polypeptide according to any one of items 2-10, wherein X₁₀ is Q.
12. VH3 binding polypeptide according to any one of items 2-10, wherein X₁₀ is Y.
13. VH3 binding polypeptide according to any one of items 2-12, wherein X₁₁ is selected from T and E.
14. VH3 binding polypeptide according to any one of items 2-12, wherein X₁₁ is selected from T and R.
15. VH3 binding polypeptide according to any one of items 2-12, wherein X₁₁ is selected from R and E.
16. VH3 binding polypeptide according to any one of items 2-14, wherein X₁₁ is T.
17. VH3 binding polypeptide according to any one of items 2-13 and 15, wherein X₁₁ is E.
18. VH3 binding polypeptide according to any one of items 2-12, 14 and 15 wherein X₁₁ is R.
19. VH3 binding polypeptide according to any one of items 2-18 wherein X₁₃ is selected from L, E, R and A; or from L, E, R and Q; or from L, E, A and Q; or from L, R, A and Q; or from E, R, A and Q; in particular selected from L, E, R and A; or from L, E, R and Q; or from L, E, A and Q; or from L, R, A and Q.
20. VH3 binding polypeptide according to any one of items 2-18 wherein X₁₃ is selected from L, E and R; or from L, E and A; or from L, E and Q; or from L, R and A; or from L, R and Q; or from L, A and Q; or from E, R and A; or from E, R and Q; or from E, A and Q; or from R, A and Q; in particular selected from L, E and R; or from L, E and A; or from L, E and Q; or from L, R and A; or from L, R and Q.
21. VH3 binding polypeptide according to any one of items 2-18 wherein X₁₃ is selected from L and E; or from L and R; or from L and A; or from L and Q; or from E and R; or from E and A; or from E and Q; or from R and A; or from R and Q; or from A and Q; in particular from L and E; or from L and R; or from L and A; or from L and Q.
22. VH3 binding polypeptide according to any one of items 2-21 wherein X₁₃ is L.
23. VH3 binding polypeptide according to any one of items 2-21 wherein X₁₃ is A.
24. VH3 binding polypeptide according to any one of items 2-21 wherein X₁₃ is E.
25. VH3 binding polypeptide according to any one of items 2-21 wherein X₁₃ is Q.
26. VH3 binding polypeptide according to any one of items 2-21 wherein X₁₃ is R.
27. VH3 binding polypeptide according to any one of items 2-26 wherein X₁₄ is selected from A, W, E, L and Q; or from A, W, E, L and R; or from A, W, E, Q and R; or from A, W, L, Q and R; or from A, E, L, Q and R; or from W, E, L, Q and R.
28. VH3 binding polypeptide according to any one of items 2-26 wherein X₁₄ is selected from A, W, E and L; or from A, W, E and Q; or from A, W, E and R; or from A, W, L and Q; or from A, W, L and R; or from A, W, Q and R; or from A, E, L and Q; or from A, E, L and R; or from A, E, Q and R; or from A, L, Q and R; or from W, E, L and Q; or from W, E, L and R; or from W, E, Q and R; or from W, L, Q and R; or from E, L, Q and R.
29. VH3 binding polypeptide according to any one of items 2-26 wherein X₁₄ is selected from A, W and E; or from A, W and L; or from A, W and Q; or from A, W and R; or from A, E and L; or from A, E and Q; or from A, E and R; or from A, L and Q; or from A, L and R; or from A, Q and R; or from W, E and L; or from W, E and Q; or from W, E and R; or from W, L and Q; or from W, L and R; or from W, Q and R; or from E, L and Q; or from E, L and R; or from E, Q and R; or from L, Q and R.
30. VH3 binding polypeptide according to any one of items 2-26 wherein X₁₄ is selected from A and W; or from A and E; or from A and L; or from A and Q; or from A and R; or from W and E; or from W and L; or from W and Q; or from W and R; or from E and L; or from E and Q; or from E and R; or from L and Q; or from L and R; or from Q and R.
31. VH3 binding polypeptide according to any one of items 2-30 wherein X₁₄ is A.
32. VH3 binding polypeptide according to any one of items 2-30 wherein X₁₄ is W.
33. VH3 binding polypeptide according to any one of items 2-30 wherein X₁₄ is E.
34. VH3 binding polypeptide according to any one of items 2-30 wherein X₁₄ is L.
35. VH3 binding polypeptide according to any one of items 2-30 wherein X₁₄ is Q.
36. VH3 binding polypeptide according to any one of items 2-30 wherein X₁₄ is R.
37. VH3 binding polypeptide according to any one of items 2-36, wherein X₁₇ is selected from A and H.
38. VH3 binding polypeptide according to any one of items 2-36, wherein X₁₇ is selected from A and L.
39. VH3 binding polypeptide according to any one of items 2-36, wherein X₁₇ is selected from H and L.
40. VH3 binding polypeptide according to any one of items 2-38, wherein X₁₇ is A.
41. VH3 binding polypeptide according to any one of items 2-37 and 39, wherein X₁₇ is H.
42. VH3 binding polypeptide according to any one of items 2-36, 38 and 39, wherein X₁₇ is L.
43. VH3 binding polypeptide according to any one of items 2-42, wherein X₁₈ is selected from R and L.
44. VH3 binding polypeptide according to any one of items 2-42, wherein X₁₈ is selected from R and H.
45. VH3 binding polypeptide according to any one of items 2-42, wherein X₁₈ is selected from H and L.
46. VH3 binding polypeptide according to any one of items 2-44, wherein X₁₈ is R.
47. VH3 binding polypeptide according to any one of items 2-43 and 45, wherein X₁₈ is L.
48. VH3 binding polypeptide according to any one of items 2-42, 44 and 45 wherein X₁₈ is H.
49. VH3 binding polypeptide according to any one of items 2-48, wherein X₂₆ is Q.
50. VH3 binding polypeptide according to any one of items 2-48, wherein X₂₆ is S.
51. VH3 binding polypeptide according to any one of items 2-50, wherein X₂₈ is N.
52. VH3 binding polypeptide according to any one of items 2-50, wherein X₂₈ is A.
53. VH3 binding polypeptide according to any one of items 2-52, wherein X₂₉ is selected from A and G.
54. VH3 binding polypeptide according to any one of items 2-53, wherein X₂₉ is A.
55. VH3 binding polypeptide according to any one of items 2-53, wherein X₂₉ is G.
56. VH3 binding polypeptide according to any one of items 2-55, wherein X₁₄ is A and X₁₀ is Y or X₉X₁₀ is selected from AY.
57. VH3 binding polypeptide according to any one of items 2-56, wherein X₉X₁₀X₁₁ is selected from the group consisting of QQT, QYT, YQT, AQE, AQR and AYR; such as the group consisting of QQT, QYT, AQE, AQR and AYR; such as the group consisting of AQE, AQR and AYR; such as the group consisting of AQR and AYR, such as wherein X₉X₁₀X₁₁ is AYR.
58. VH3 binding polypeptide according to any one of items 2-57, wherein X₁₇X₁₈ is selected from the group consisting of AR, HL and LH; such as the group consisting of AR and HL; such as wherein X₁₇X₁₈ is AR.
59. VH3 binding polypeptide according to any one of items 2-58, wherein X₂₄X₂₅ is EE.
60. VH3 binding polypeptide according to any one of items 2-59, wherein X₂₈X₂₉ is selected from the group consisting of NA, NG, AA and AG; such as the group consisting of NA, NG and AG or the group consisting of NG, AA and AS; such as the group consising of NG and AG or the group consisting of AG and NA or the group consisting of AG and AA.
61. VH3 binding polypeptide according to any one of items 2-60, wherein X₂₈X₂₉ is NA or AG or AA or NG.
62. VH3 binding polypeptide according to any one of items 2-61, wherein independently of each other X₉X₁₀X₁₁ is selected from the group consisting of AQE, AQR and AYR; and X₁₃X₁₄ is selected from the group consisting of LA, LW, AA, AE, AL, AQ, AR, EA, EE, EL, EQ, ER, LA, LE, LL, LQ, LR, QA, QE, QL, QQ, QR, RA, RE, RL, RQ and RR;
such as wherein independently of each other X₉X₁₀X₁₁ is selected from the group consisting of AQE, AQR and AYR; and X₁₃X₁₄ is selected from the group consisting of LA, AA, AE, AL, AQ, AR, EA, EE, EL, EQ, ER, LA, LE, LL, LQ, LR, QA, QE, QL, QQ, QR, RA, RE, RL, RQ and RR;
such as wherein independently of each other X₉X₁₀X₁₁ is selected from the group consisting of AQR and AYR; and X₁₃X₁₄ is selected from the group consisting of LA, AA, AE, AL, AQ, AR, EA, EE, EL, EQ, ER, LA, LE, LL, LQ, LR, QA, QE, QL, QQ, QR, RA, RE, RL, RQ and RR; such as wherein X₉X₁₀X₁₁ is AYR and X₁₃X₁₄ is selected from the group consisting of LA, AA, AE, AL, AQ, AR, EA, EE, EL, EQ, ER, LA, LE, LL, LQ, LR, QA, QE, QL, QQ, QR, RA, RE, RL, RQ and RR.
63. VH3 binding polypeptide according to any one of items 2-62, wherein X₉X₁₀X₁₁X₁₃X₁₄ is selected from the group consisting of QQTLA, QYTLA, YQTLA, AQELA, AQRLA, AYRLA and AYRLW; such as the group consisting of QQTLA, QYTLA, AQELA, AQRLA, AYRLA and AYRLW; such as the group consisting of AQELA, AQRLA, AYRLA and AYRLW; such as the group consisting of AQRLA and AYRLA; such as wherein X₉X₁₀X₁₁X₁₃X₁₄ is AYRLA.
64. VH3 binding polypeptide according to any one of items 2-63, wherein independently of each other X₉X₁₀X₁₁ is selected from the group consisting of AQE, AQR and AYR; and X₁₃X₁₄ is selected from the group consisting of LA, AA, AE, AL, AQ, AR, EA, EE, EL, EQ, ER, LA, LE, LL, LQ, LR, QA, QE, QL, QQ, QR, RA, RE, RL, RQ, RR and LW; and X₁₇X₁₈ is selected from LH and AR;
such as wherein independently of each other X₉X₁₀X₁₁ is selected from the group consisting of AQR and AYR; and X₁₃X₁₄ is selected from the group consisting of LA, AA, AE, AL, AQ, AR, EA, EE, EL, EQ, ER, LA, LE, LL, LQ, LR, QA, QE, QL, QQ, QR, RA, RE, RL, RQ, RR and LW; and X₁₇X₁₈ is selected from LH and AR;
such as wherein independently of each other X₉X₁₀X₁₁ is AYR; and X₁₃X₁₄ is selected from the group consisting of LA, AA, AE, AL, AQ, AR, EA, EE, EL, EQ, ER, LA, LE, LL, LQ, LR, QA, QE, QL, QQ, QR, RA, RE, RL, RQ, RR and LW; and X₁₇X₁₈ is selected from LH and AR;
such as wherein independently of each other X₉X₁₀X₁₁ is AYR; and X₁₃X₁₄ is selected from the group consisting of LA, AA, AE, AL, AQ, AR, EA, EE, EL, EQ, ER, LA, LE, LL, LQ, LR, QA, QE, QL, QQ, QR, RA, RE, RL, RQ, RR and LW; and X₁₇X₁₈ is AR.
65. VH3 binding polypeptide according to any one of items 2-64, wherein X₉X₁₀X₁₁X₁₃X₁₄X₁₇X₁₈ is selected from the group consisting of
QQTLALH, QYTLALH, YQTLALH, QQTLAAR, AQELALH, AQRLALH, AYRLALH, AYRLWLH, AYRLWAR, AYRLAAR, AYRLAHL and AYRLWHL; such as the group consising of QQTLALH, QYTLALH, QQTLAAR, AQELALH, AQRLALH, AYRLALH, AYRLWLH, AYRLWAR, AYRLAAR, AYRLAHL and AYRLWHL; such as the group consiting of AQELALH, AQRLALH, AYRLALH, AYRLWAR, AYRLAAR and AYRLAHL; such as the group consisting of, AQRLALH, AYRLALH and AYRLAAR; such as the group consisting of AQRLALH and AYRLAAR.
66. VH3 binding polypeptide according to any one of items 2-65, wherein X₉X₁₀X₁₁X₁₃X₁₄X₁₇X₁₈ is AYRLAAR.
67. VH3 binding polypeptide according to any one of items 2-65, wherein X₂₄X₂₅X₂₈X₂₉ is selected from the group consisting of EENA, EENG, EEAA and EEAG; such as the group consisting of EENA, EENG and EEAG; such as the group consisting of EENG and EEAG; such as wherein X₂₄X₂₅X₂₈X₂₉ is EEAG.
68. VH3 binding polypeptide according to any one of items 2-67, wherein X₂₆X₂₈X₂₉ is selected from the group consisting of QNG, QAA, QAG, QNA, and SAG; such as the group consisting of QNG, QAG, QNA and SAG; such as the group consisting of QNG, QAG and SAG and or the group consisting QNG, QAG, QNA or the group consisting of QNG, QNA and SAG; such as the group consisting of QAG and SAG; such as wherein X₂₆X₂₈X₂₉ is QAG or SAG.
69. VH3 binding polypeptide according to any one of items 2-68, wherein X₂₆X₂₈X₂₉ is selected from the group consisting of QAG and SAG; such as wherein X₂₆X₂₈X₂₉ is QAG.
70. VH3 binding polypeptide according to any one of items 2-69, wherein sequence i) corresponds to the sequence from position 8 to position 36 in a sequence selected from the group consisting of SEQ ID NO:1-82, such as the group consisting of SEQ ID NO:1-40 and 42-81, such as the group consisting of SEQ ID NO:1-15 and 42-56.
71. VH3 binding polypeptide according to item 70, wherein sequence i) corresponds to the sequence from position 8 to position 36 in a sequence selected from the group consisting of SEQ ID NO:1-41, such as the group consisting of SEQ ID NO:1-40, such as the group consisting of 1-15.
72. VH3 binding polypeptide according to item 70, wherein sequence i) corresponds to the sequence from position 8 to position 36 in a sequence selected from the group consisting of SEQ ID NO:1, 2, 4-43 and 45-82; such as the group consisting of SEQ ID NO:5-7, 9-11, 13-41, 46-48, 50-52 and 54-82; such as the group consisting of SEQ ID NO:6-7, 10, 13-41, 47-48, 51 and 54-82; such as the group consisting of SEQ ID NO:6-7, 10, 13, 15-41, 47-48, 51, 54 and 56-82; such as the group consisting of SEQ ID NO:6, 10, 13, 15-41, 47, 51, 54 and 56-82; such as the group consisting of SEQ ID NO:13, 15-41, 54 and 56-82; such as the group consisting of SEQ ID NO:13, 15-40, 54 and 56-81; such as the group consisting of SEQ ID NO:13, 15, 54 and 56.
73. VH3 binding polypeptide according to item 70, wherein sequence i) corresponds to the sequence from position 8 to position 36 in a sequence selected from the group consisting of SEQ ID NO:15-41 and 56-82; such as the group consisting of SEQ ID NO:15-40 and 56-81 or the group consisting of SEQ ID NO:15, 41, 56 and 82.
74. VH3 binding polypeptide according to item 70, wherein sequence i) corresponds to the sequence from position 8 to position 36 in a sequence selected from the group consisting of SEQ ID NO:14, 15, 55 and 56.
75. VH3 binding polypeptide according to item 70, wherein sequence i) corresponds to the sequence from position 8 to position 36 in a sequence selected from the group consisting of SEQ ID NO:1, 2, 4-41; such as the group consisting of SEQ ID NO:5-7, 9-11, 13-41; such as the group consisting of SEQ ID NO:6-7, 10, 13-41; such as the group consisting of SEQ ID NO:6-7, 10, 13, 15-41; such as the group consisting of SEQ ID NO:6, 10, 13, 15-41; such as the group consisting of SEQ ID NO:13, 15-41; such as the group consisting of SEQ ID NO:13, 15-40; such as the group consisting of SEQ ID NO:13 and 15.
76. VH3 binding polypeptide according to item 70, wherein sequence i) corresponds to the sequence from position 8 to position 36 in a sequence selected from the group consisting of SEQ ID NO:15-41; such as the group consisting of SEQ ID NO:15-40 or the group consisting of SEQ ID NO:15 and 41.
77. VH3 binding polypeptide according to item 70, wherein sequence i) corresponds to the sequence from position 8 to position 36 in a sequence selected from the group consisting of SEQ ID NO:14 and 15.
78. VH3 binding polypeptide according to item 70, wherein sequence i) corresponds to the sequence from position 8 to position 36 in a sequence selected from the group consisting of SEQ ID NO:42-43 and 45-82; such as the group consisting of SEQ ID NO:46-48, 50-52 and 54-82; such as the group consisting of SEQ ID NO:47-48, 51 and 54-82; such as the group consisting of SEQ ID NO:47-48, 51, 54 and 56-82; such as the group consisting of SEQ ID NO:47, 51, 54 and 56-82; such as the group consisting of SEQ ID NO:54 and 56-82; such as the group consisting of SEQ ID NO:54 and 56-81; such as the group consisting of SEQ ID NO:54 and 56.
79. VH3 binding polypeptide according to item 70, wherein sequence i) corresponds to the sequence from position 8 to position 36 in a sequence selected from the group consisting of SEQ ID NO:56-82; such as the group consisting of SEQ ID NO 56-81 or the group consisting of SEQ ID NO:56 and 82.
80. VH3 binding polypeptide according to item 70, wherein sequence i) corresponds to the sequence from position 8 to position 36 in a sequence selected from the group consisting of SEQ ID NO:55 and 56.
81. VH3 binding polypeptide according to item 70, wherein sequence i) corresponds to the sequence from position 8 to position 36 in SEQ ID NO:13, SEQ ID NO:14 or SEQ ID NO:15.
82. VH3 binding polypeptide according to item 70, wherein sequence i) corresponds to the sequence from position 8 to position 36 in SEQ ID NO:54, SEQ ID NO:55 or SEQ ID NO:56.
83. VH3 binding polypeptide according to any one of items 2-4, having a sequence iii) selected from group consisting of a sequence corresponding to residues 8-36 in SEQ ID NO:83; a sequence corresponding to residues 8-36 in SEQ ID NO:84; a sequence corresponding to residues 8-36 in SEQ ID NO:85; a sequence corresponding to residues 11-39 in SEQ ID NO:86; and a sequence corresponding to residues 1-29 in SEQ ID NO:87.
84. VH3 binding polypeptide according to any one of items 1-83, wherein said polypeptide derived from SpA or said Sequence A forms part of a three-helix bundle protein domain.
85. VH3 binding polypeptide according to item 84, wherein said polypeptide derived from SpA or said Sequence A forms part of two helices with an interconnecting loop, within said three-helix bundle protein domain.
86. VH3 binding polypeptide according to any one of items 84-85, wherein said three-helix bundle protein domain is selected from bacterial receptor domains.
87. VH3 binding polypeptide according to any one of items 84-86, wherein said three-helix bundle protein domain is selected from domains of protein A from Staphylococcus aureus or derivatives thereof.
88. VH3 binding polypeptide according to one of items 2-87, further comprising a Sequence B arranged C-terminally of said sequence A, which Sequence B consists of an amino acid sequence selected from iv) and v), and wherein iv) and v) are defined as follows:

iv)
(SEQ ID NO: 96)
DPSX₄₀SX₄₂X₄₃X₄₄LX₄₆EAX₄₉X₅₀LNX₅₃X₅₄

wherein, independently from each other,
X₄₀ is selected from Q, T and V;
X₄₂ is selected from A, K, L and T;
X₄₃ is selected from N, E, A, and S;
X₄₄ is selected from L, I and V;
X₄₆ is selected from A, G, S and K, such as from A, S and K;
X₄₉ is selected from K and Q, such as is K;
X₅₀ is selected from K and R;
X₅₃ is selected from D, E and K; and
X₅₄ is selected from A and S;
v) an amino acid sequence which has at least 75% identity to a sequence defined by iv).
89.VH3 binding polypeptide according to any one of items 2-88, comprising a binding module sequence C, which Sequence C consists of Sequence A according to any one of items 2-87 and Sequence B according to item 88, in the following order from the N-terminus to the C-terminus
[Sequence A]-[Sequence B]
   or any amino acid sequence which has at least 70% identity to any sequence selected from the group consisting of: residues 8-54 in SEQ ID NO:88, residues 8-54 in SEQ ID NO:89, residues 8-54 in SEQ ID NO:90, residues 8-54 in SEQ ID NO:91, residues 8-54 in SEQ ID NO:92, residues 11-57 in SEQ ID NO:93 and residues 1-47 in SEQ ID NO:94.
90. VH3 binding polypeptide in according to any one of items 2-89, comprising a binding module Sequence C, which Sequence C consists of the sequences [Sequence A], [L2], [H3], in the following order from the N-terminus to the C-terminus
[Sequence A]-[L2]-[H3]
   wherein [Sequence A] is as defined in any one of items 2-87,
   and wherein, independently from each other, [L2] is selected from the group consisting of:

(SEQ ID NO: 97)
DPSV;
(SEQ ID NO: 98)
DPSQ;
(SEQ ID NO: 99)
EPSQ;
(SEQ ID NO: 100)
DPST;
(SEQ ID NO: 101)
EPSV;
and
(SEQ ID NO: 102)
DPSL;

and [H3] is selected from the group consisting of:

(SEQ ID NO: 103)
SANLLAEAKKLNDA;
(SEQ ID NO: 104)
SKEILAEAKKLNDA;
(SEQ ID NO: 105)
SKAILAEAKKLNDA;
(SEQ ID NO: 106)
SANLLAEAKKLNDA;
(SEQ ID NO: 107)
SANLLAEAKKLNES;
(SEQ ID NO: 108)
STNVLGEAKKLNES;
(SEQ ID NO: 109)
SANVLGEAQKLNDS;
(SEQ ID NO: 110)
SLEILCEAKKLNDA;
(SEQ ID NO: 111)
SLEILAEAKKLNDA;
and
(SEQ ID NO: 112)
SKKILKEAKKLNKA.

91. VH3 binding polypeptide according to any one of items 89-90, comprising a binding module Sequence C selected from the group consisting of

(SEQ ID NO: 113)
[Sequence A]-DPSQSANLLAEAKKLNDA;
(SEQ ID NO: 114)
[Sequence A]-DPSVSKEILAEAKKLNDA;
(SEQ ID NO: 115)
[Sequence A]-DPSVSKAILAEAKKLNDA;
(SEQ ID NO: 116)
[Sequence A]-DPSQSANLLAEAKKLNES;
(SEQ ID NO: 117)
[Sequence A]-DPSVSLEILGEAKKLNDA;
(SEQ ID NO: 118)
[Sequence A]-DPSVSLEILCEAKKLNDA;
(SEQ ID NO: 119)
[Sequence A]-DPSVSLEILAEAKKLNDA;
(SEQ ID NO: 120)
[Sequence A]-DPSVSLALLAEAKKLNDA;
(SEQ ID NO: 121)
[Sequence A]-DPSQSANLLSEAKKLNES;
(SEQ ID NO: 122)
[Sequence A]-DPSQSTNVLGEAKKLNES;
and
(SEQ ID NO: 123)
[Sequence A]-DPSQSANVLGEAQKLNDS;

wherein [Sequence A] is as defined in any one of items 2-87.
92. VH3 binding polypeptide according to any one of items 88-91, comprising a binding module Sequence C selected from the group consisting of:

vi)
(SEQ ID NO: 113)
[Sequence A]-DPSQSANLLAEAKKLNDA;

wherein [Sequence A] is as defined in any one of items 2-87; and
vii) an amino acid sequence which has at least 85% identity to the sequence defined in vi).
93. VH3 binding polypeptide according to any one of items 88-91, comprising a binding module Sequence C selected from the group consisting of:

viii)
(SEQ ID NO: 115)
[Sequence A]-DPSVSKAILAEAKKLNDA

wherein [Sequence A] is as defined in any one of items 2-87; and
ix) an amino acid sequence which has at least 85% identity to the sequence defined in viii).
94. VH3 binding polypeptide according to any one of items 88-91, comprising a binding module Sequence C selected from the group consisting of:

x)
(SEQ ID NO: 114)
[Sequence A]-DPSVSKEILAEAKKLNDA

wherein [Sequence A] is as defined in any one of items 2-87; and
xi) an amino acid sequence which has at least 85% identity to the sequence defined in x).
95. VH3 binding polypeptide according to any one of items 2-95, wherein sequence vi) or viii) corresponds to the sequence from position 8 to position 54 in a sequence selected from the group consisting of SEQ ID NO:1-82, such as the group consisting of SEQ ID NO:1-40 and 42-81, such as the group consisting of SEQ ID NO:1-15 and 42-56.
96. VH3 binding polypeptide according to item 95, wherein sequence viii) corresponds to the sequence from position 8 to position 54 in a sequence selected from the group consisting of SEQ ID NO:1-41, such as the group consisting of SEQ ID NO:1-40, such as the group consisting of SEQ ID NO:1-15.
97. VH3 binding polypeptide according to item 95, wherein sequence vi) or viii) corresponds to the sequence from position 8 to position 54 in a sequence selected from the group consisting of SEQ ID NO:1, 2, 4-43 and 45-82; such as the group consisting of SEQ ID NO:5-7, 9-11, 13-41, 46-48, 50-52 and 54-82; such as the group consisting of SEQ ID NO:6-7, 10, 13-41, 47-48, 51 and 54-82; such as the group consisting of SEQ ID NO:6-7, 10, 13, 15-41, 47-48, 51, 54 and 56-82; such as the group consisting of SEQ ID NO:6, 10, 13, 15-41, 47, 51, 54 and 56-82; such as the group consisting of SEQ ID NO:13, 15-41, 54 and 56-82; such as the group consisting of SEQ ID NO:13, 15-40, 54 and 56-81; such as the group consisting of SEQ ID NO:13, 15, 54 and 56.
98. VH3 binding polypeptide according to item 95, wherein sequence vi) or viii) corresponds to the sequence from position 8 to position 54 in a sequence selected from the group consisting of SEQ ID NO:15-41 and 56-82; such as the group consisting of SEQ ID NO:15-40 and 56-81 or the group consisting of SEQ ID NO:15, 41, 56 and 82.
99. VH3 binding polypeptide according to item 95, wherein sequence vi) or viii) corresponds to the sequence from position 8 to position 54 in a sequence selected from the group consisting of SEQ ID NO:14, 15, 55 and 56.
100. VH3 binding polypeptide according to item 95, wherein sequence viii) corresponds to the sequence from position 8 to position 54 in a sequence selected from the group consisting of SEQ ID NO:1, 2, 4-41; such as the group consisting of SEQ ID NO:5-7, 9-11, 13-41; such as the group consisting of SEQ ID NO:6-7, 10, 13-41; such as the group consisting of SEQ ID NO:6-7, 10, 13, 15-41; such as the group consisting of SEQ ID NO:6, 10, 13, 15-41; such as the group consisting of SEQ ID NO:13, 15-41; such as the group consisting of SEQ ID NO:13, 15-40; such as the group consisting of SEQ ID NO:13 and 15.
101. VH3 binding polypeptide according to item 95, wherein sequence viii) corresponds to the sequence from position 8 to position 54 in a sequence selected from the group consisting of SEQ ID NO:15-41; such as the group consisting of SEQ ID NO:15-40 or the group consisting of SEQ ID NO:15 and 41.
102. VH3 binding polypeptide according to item 95, wherein sequence viii) corresponds to the sequence from position 8 to position 54 in a sequence selected from the group consisting of SEQ ID NO:14 and 15.
103. VH3 binding polypeptide according to item 95, wherein sequence vi) corresponds to the sequence from position 8 to position 54 in a sequence selected from the group consisting of SEQ ID NO:42-43 and 45-82; such as the group consisting of SEQ ID NO:46-48, 50-52 and 54-82; such as the group consisting of SEQ ID NO:47-48, 51 and 54-82; such as the group consisting of SEQ ID NO:47-48, 51, 54 and 56-82; such as the group consisting of SEQ ID NO:47, 51, 54 and 56-82; such as the group consisting of SEQ ID NO:54 and 56-82; such as the group consisting of SEQ ID NO:54 and 56-81; such as the group consisting of SEQ ID NO:54 and 56.
104. VH3 binding polypeptide according to item 95, wherein sequence vi) corresponds to the sequence from position 8 to position 54 in a sequence selected from the group consisting of SEQ ID NO:56-82; such as the group consisting of SEQ ID NO 56-81 or the group consisting of SEQ ID NO:56 and 82.
105. VH3 binding polypeptide according to item 95, wherein sequence vi) corresponds to the sequence from position 8 to position 54 in a sequence selected from the group consisting of SEQ ID NO:55 and 56.
106. VH3 binding polypeptide according to item 95, wherein sequence viii) corresponds to the sequence from position 8 to position 54 in SEQ ID NO:13, SEQ ID NO:14 or SEQ ID NO:15.
107. VH3 binding polypeptide according to item 95, wherein sequence vi) corresponds to the sequence from position 8 to position 54 in SEQ ID NO:54, SEQ ID NO:55 or SEQ ID NO:56.
108. VH3 binding polypeptide according to item 83, comprising a sequence selected from group consisting of a sequence corresponding to residues 8-54 in SEQ ID NO:83; a sequence corresponding to residues 8-54 in SEQ ID NO:84; a sequence corresponding to residues 8-54 in SEQ ID NO:85; a sequence corresponding to residues 11-57 in SEQ ID NO:86 and residues 1-47 in SEQ ID NO:87.
109. VH3 binding polypeptide according to any one of items 2-82 and 84-107, comprising a sequence [S1], [binding module Sequence C] and [S2] in the following order from the N-terminus to the C-terminus
[S1]-[binding module Sequence C]-[S2],
   wherein [S1] or [S2] may be present or absent and [binding module sequence C] is as defined in any one of items 88-107, and
   wherein, independently from each other,
[S1] is selected from the group consisting of

(SEQ ID NO: 124)
ADNKFNK;
(SEQ ID NO: 125)
VDAKFDK;
(SEQ ID NO: 126)
VDNKFNK;
(SEQ ID NO: 127)
ADAQQNKFNK;
(SEQ ID NO: 128)
IAAKHDK;
(SEQ ID NO: 129)
IAAQHDK;
(SEQ ID NO: 130)
ADNNFNK;
(SEQ ID NO: 131)
IAAKFDE;
(SEQ ID NO: 132)
PAAKHDK;
(SEQ ID NO: 133)
ADNAFNT;
FNK;
(SEQ ID NO: 134)
ADNRFNE;
(SEQ ID NO: 135)
IDSKFDE;
(SEQ ID NO: 136)
ADNRFNR;
(SEQ ID NO: 137)
ADNKHNK;
(SEQ ID NO: 138)
ADSKFDE;
(SEQ ID NO: 139)
IDAKHDE;
(SEQ ID NO: 140)
QQNKFNK;
(SEQ ID NO: 141)
ADNKFHK;
(SEQ ID NO: 142)
KFNK;
(SEQ ID NO: 143)
ADNNFNR;
(SEQ ID NO: 144)
AAAKHDK;
(SEQ ID NO: 145)
IDNKFNK;
(SEQ ID NO: 146)
IDAKFDE;
(SEQ ID NO: 147)
DNNFNK;
(SEQ ID NO: 148)
ADNKFNE;
(SEQ ID NO: 149)
AAAQHDK;
and
(SEQ ID NO: 150)
AAAKFDE;

and [S2] is selected from the group consisting of

(SEQ ID NO: 151)
QAPK;
(SEQ ID NO: 152)
QAPP;
QAP;
(SEQ ID NO: 153)
QAPR;
(SEQ ID NO: 154)
QAPE;
APK;
(SEQ ID NO: 155)
QAPG;
(SEQ ID NO: 156)
QAIK;
and
QA.

110. VH3 binding polypeptide according to any one of items 2-109, comprising a sequence selected from the group consisting of:

(SEQ ID NO: 157)
ADNKFNK-[Sequence A]-DPSQSANLLAEAKKLNDAQAPK;
(SEQ ID NO: 158)
VDNKFNK-[Sequence A]-DPSQSANLLAEAKKLNDAQAPK;
(SEQ ID NO: 159)
ADNKFNK-[Sequence A]-DPSVSKEILAEAKKLNDAQAPK;
(SEQ ID NO: 160)
VDAKFDK-[Sequence A]-DPSQSANLLAEAKKLNDAQAPK;
(SEQ ID NO: 161)
VDNKFNK-[Sequence A]-DPSVSKEILAEAKKLNDAQAPK;
(SEQ ID NO: 162)
VDAKFDK-[Sequence A]-DPSVSKAILAEAKKLNDAQAPK;
(SEQ ID NO: 163)
ADNNFNK-[Sequence A]-DPSQSANLLAEAKKLNESQAPK;
(SEQ ID NO: 164)
ADNKHNK-[Sequence A]-DPSQSANLLAEAKKLNDAQAPK;
(SEQ ID NO: 165)
ADNKFNK-[Sequence A]-DPSQSANLLAEAAPK;
(SEQ ID NO: 166)
IAAQHDK-[Sequence A]-DPSVSLEILAEAKKLNDAQAPK;
(SEQ ID NO: 167)
ADNKFHK-[Sequence A]-DPSQSANLLAEAKKLNDAQAPK;
(SEQ ID NO: 168)
FNK-[Sequence A]-DPSQSANLLAEAKKLNDAQAPK;
(SEQ ID NO: 169)
IAAQHDK-[Sequence A]-DPSVSLEILCEAKKLNDAQAPK;
(SEQ ID NO: 170)
IDAKFDE-[Sequence A]-DPSVSLALLAEAKKLNDAQAPP;
(SEQ ID NO: 171)
DNNFNK-[Sequence A]-DPSQSANLLAEAKKLNESQAPK;
(SEQ ID NO: 172)
VDAKFDK-[Sequence A]-DPSQSANLLAEAKKLNDAQAIK;
(SEQ ID NO: 173)
ADNRFNE-[Sequence A]-DPSVSKEILAEAKKLNDAQAPE;
and
(SEQ ID NO: 174)
IDAKFDE-[Sequence A]-DPSVSLSLLAEAKKLNDAQAPP;

wherein [Sequence A] is as defined in any one of items 2-87.
111. VH3 binding polypeptide according to any one of items 2-110, comprising a sequence selected from the group consisting of:

xii)
(SEQ ID NO: 158)
VDNKFNK-[Sequence A]-DPSQSANLLAEAKKLNDAQAPK

wherein [Sequence A] is as defined in any one of items 2-87;
xiii) an amino acid sequence which has at least 86% identity to the sequence defined in xii).
112. VH3 binding polypeptide according to any one of items 2-110, comprising a sequence selected from the group consisting of:

xiv)
(SEQ ID NO: 162)
VDAKFDK-[Sequence A]-DPSVSKAILAEAKKLNDAQAPK

wherein [Sequence A] is as defined in any one of items 2-87; and
xv) an amino acid sequence which has at least 86% identity to the sequence defined in xiv).
113. VH3 binding polypeptide according to any one of items 2-112, wherein sequence xii) or xiv) corresponds to a sequence selected from the group consisting of SEQ ID NO:1-82, such as the group consisting of SEQ ID NO:1-40 and 42-81, such as the group consisting of SEQ ID NO:1-15 and 42-56.
114. VH3 binding polypeptide according to item 113, wherein sequence xiv) corresponds to a sequence selected from the group consisting of SEQ ID NO:1-41, such as the group consisting of SEQ ID NO:1-40, such as the group consisting of SEQ ID NO:1-15.
115. VH3 binding polypeptide according to item 113, wherein sequence xii) or xiv) corresponds to a sequence selected from the group consisting of SEQ ID NO:1, 2, 4-43 and 45-82; such as the group consisting of SEQ ID NO:5-7, 9-11, 13-41, 46-48, 50-52 and 54-82; such as the group consisting of SEQ ID NO:6-7, 10, 13-41, 47-48, 51 and 54-82; such as the group consisting of SEQ ID NO:6-7, 10, 13, 15-41, 47-48, 51, 54 and 56-82; such as the group consisting of SEQ ID NO:6, 10, 13, 15-41, 47, 51, 54 and 56-82; such as the group consisting of SEQ ID NO:13, 15-41, 54 and 56-82; such as the group consisting of SEQ ID NO:13, 15-40, 54 and 56-81; such as the group consisting of SEQ ID NO:13, 15, 54 and 56.
116. VH3 binding polypeptide according to item 113, wherein sequence xii) or xiv) corresponds to a sequence selected from the group consisting of SEQ ID NO:15-41 and 56-82; such as the group consisting of SEQ ID NO:15-40 and 56-81 or the group consisting of SEQ ID NO:15, 41, 56 and 82.
117. VH3 binding polypeptide according to item 113, wherein sequence xii) or xiv) corresponds to a sequence selected from the group consisting of SEQ ID NO:14, 15, 55 and 56.
118. VH3 binding polypeptide according to item 113, wherein sequence xiv) corresponds to a sequence selected from the group consisting of SEQ ID NO:1, 2, 4-41; such as the group consisting of SEQ ID NO:5-7, 9-11, 13-41; such as the group consisting of SEQ ID NO:6-7, 10, 13-41; such as the group consisting of SEQ ID NO:6-7, 10, 13, 15-41; such as the group consisting of SEQ ID NO:6, 10, 13, 15-41; such as the group consisting of SEQ ID NO:13, 15-41; such as the group consisting of SEQ ID NO:13, 15-40; such as the group consisting of SEQ ID NO:13 and 15.
119. VH3 binding polypeptide according to item 113, wherein sequence xiv) corresponds to a sequence selected from the group consisting of SEQ ID NO:15-41; such as the group consisting of SEQ ID NO:15-40 or the group consisting of SEQ ID NO:15 and 41.
120. VH3 binding polypeptide according to item 113, wherein sequence xiv) corresponds to a sequence selected from the group consisting of SEQ ID NO:14 and 15.
121. VH3 binding polypeptide according to item 113, wherein sequence xii) corresponds to a sequence selected from the group consisting of SEQ ID NO:42-43 and 45-82; such as the group consisting of SEQ ID NO:46-48, 50-52 and 54-82; such as the group consisting of SEQ ID NO:47-48, 51 and 54-82; such as the group consisting of SEQ ID NO:47-48, 51, 54 and 56-82; such as the group consisting of SEQ ID NO:47, 51, 54 and 56-82; such as the group consisting of SEQ ID NO:54 and 56-82; such as the group consisting of SEQ ID NO:54 and 56-81; such as the group consisting of SEQ ID NO:54 and 56.
122. VH3 binding polypeptide according to item 113, wherein sequence xii) corresponds to a sequence selected from the group consisting of SEQ ID NO:56-82; such as the group consisting of SEQ ID NO 56-81 or the group consisting of SEQ ID NO:56 and 82.
123. VH3 binding polypeptide according to item 113, wherein sequence xii) corresponds to a sequence selected from the group consisting of SEQ ID NO:55 and 56.
124. VH3 binding polypeptide according to item 113, wherein sequence xiv) corresponds to in SEQ ID NO:13, SEQ ID NO:14 or SEQ ID NO:15.
125. VH3 binding polypeptide according to item 113, wherein sequence xii) corresponds to a sequence in SEQ ID NO:54, SEQ ID NO:55 or SEQ ID NO:56.
126. VH3 binding polypeptide according to item 108, comprising a sequence selected from group consisting of a sequence corresponding to residues 1-58 in SEQ ID NO:83; a sequence corresponding to residues 1-58 in SEQ ID NO:84; a sequence corresponding to residues 1-58 in SEQ ID NO:85; a sequence corresponding to residues 3-60 in SEQ ID NO:86 and a sequence corresponding to residues 1-51 in SEQ ID NO:87 or selected from group consisting of a sequence corresponding to residues 1-58 in SEQ ID NO:83; a sequence corresponding to residues 1-58 in SEQ ID NO:84; a sequence corresponding to residues 1-58 in SEQ ID NO:85; a sequence corresponding to residues 1-63 in SEQ ID NO:86 and a sequence corresponding to residues 1-51 in SEQ ID NO:87.
127. VH3 binding polypeptide multimer, wherein each monomer of the multimer comprises a VH3 binding polypeptide which is independently selected from any VH3 binding polypeptide defined in any one of items 1-126.
128. VH3 binding polypeptide multimer, wherein each monomer of the multimer comprises a Sequence A which is independently selected from any Sequence A defined in any one of items 2-87.
129. VH3 binding polypeptide multimer according to item 127 or 128, wherein said multimer is selected from the group consisting of dimer, trimer, tetramer, pentamer, hexamer, heptamer, octamer, nonamer and decamer.
130. VH3 binding polypeptide multimer according to any one of items 127-129, wherein said multimer is selected from the group consisting of dimer, tetramer, pentamer, hexamer, heptamer and octamer; such as the group consisting of dimer, tetramer and hexamer.
131. VH3 binding polypeptide multimer according to any one of items 127-130, wherein said multimer is a tetramer or a hexamer.
132. VH3 binding polypeptide multimer according to any one of items 127-131, wherein said multimer is a homomer.
133. VH3 binding polypeptide multimer according to any one of items 127-132, wherein said multimer comprises a sequence selected from the group consiting of SEQ ID NO:175, SEQ ID NO:176 and SEQ ID NO:177, such as wherein said sequence is SEQ ID NO:177.
134. VH3 binding polypeptide multimer according to any one of items 127-132, wherein said multimer is a heteromer comprising at least two or at least three different VH3 binding polypeptides each defined as in any one of items 1-126.
135. VH3 binding polypeptide multimer according to any one of items 127-131 and 134, wherein said multimer is a heteromer comprising at least two or at least three different sequence A, each defined as in any one of items 2-87.
136. VH3 binding polypeptide multimer according to any one of items 127-135, further comprising at least one linker.
137. VH3 binding polypeptide multimer according to item 136, wherein said linker comprises up to 15 amino acid residues.
138. VH3 binding polypeptide according to any one of item 1-126 or a VH3 binding polypeptide multimer according to any one of items 127-137 which comprises additional amino acids at the C-terminal and/or N-terminal end.
139. VH3 binding polypeptide or VH3 binding polypeptide multimer according to item 138, wherein said additional amino acid(s) improve(s) production, purification, stabilization in vitro or coupling of the polypeptide.
140. VH3 binding polypeptide or VH3 binding polypeptide multimer according to item 138 or 139, wherein said additional amino acid(s) improve(s) coupling of the polypeptide and are selected from the group consisting of one or more cysteine residues, a plurality of lysine residues and a plurality of histidine residues.
141. VH3 binding polypeptide multimer according to any one of items 127-140, wherein said VH3 binding polypeptide monomers are expressed as a fusion protein.
142. VH3 binding polypeptide multimer according to any one of items 127-140, wherein said VH3 binding polypeptide monomer units are covalently coupled together.
143. VH3 binding polypeptide according to any one of items 1-126 and 138-140 or VH3 binding polypeptide multimer according to any one of items 127-142, which is capable of binding to VH3 such that the K_{D} value of the interaction is at most 1×10⁻⁷ M, such as at most 1×10⁻⁸ M, such as at most 1×10⁻⁹ M, such as at most 1×10⁻¹⁰ M, such as at most 1×10⁻¹¹ M.
144. VH3 binding polypeptide according to any one of items 1-126, 138-140 and 143 or VH3 binding polypeptide multimer according to any one of items 127-143, which is capable of binding to VH3 such as that the K_{D} value of the interaction is at least 25%, such as at least 30%, such as at least 40%, such as at least 50%, such as at least 60%, such as at least 70% such as at least 80%, such as at least 90% of the K_{D} value of the interaction of SEQ ID NO:88 and/or SEQ ID NO:89 with VH3, such as of the of the interaction of SEQ ID NO:88 with VH3.
145. VH3 binding polypeptide according to any one of items 1-126, 138-140 and 143-144 or VH3 binding polypeptide multimer according to any one of items 127-144, which is not capable of binding to said Fc region with a K_{D} value of the interaction of less than 1×10⁻⁴ M, such as less than 1×10⁻³ M.
146. VH3 binding polypeptide according to any one of items 1-126, 138-140 and 143-144 or VH3 binding polypeptide multimer according to any one of items 127-145, which retains at least 19%, such as at least 20%, such as at least 21%, such as at least 23%, such as at least 25%, such as at least 27%, such as at least 29%, such as at least 31%, such as at least 33%, such as at least 35%, such as at least 37%, such as at least 39%, such as at least 41%, such as at least 43%, such as at least 45%, such as at least 47%, such as at least 48%, such as at least 50% of its original VH3 binding capability after 100 repeated binding cycles followed by cleaning with 0.5 M NaOH.
147. VH3 binding polypeptide according to any one of items 1-126, 138-140 and 143-146 or VH3 binding polypeptide multimer according to any one of items 127-146, which exhibits an alkali clean stability of at least 35%, such as at least 40%, such as at least 40%, such as at least 45%, such as at least 50%, such as at least 55%, such as at least 60%, such as at least 65%, such as at least 70%, such as at least 75%, such as at least 80%, such as at least 85%, such as at least 90%, such as at least 95% of the alkali clean stability of SEQ ID NO:88 or of SEQ ID NO:89 after incubation in 0.5 M NaOH, such as after repeated incubation with 0.5 M NaOH.
148. VH3 binding polypeptide according to any one of items 1-126, 138-140 and 143-147 or VH3 binding polypeptide multimer according to any one of items 127-147, which exhibits an alkali clean stability in the range of ±25%, such as in the range of +20%, such as in the range of ±15%, such as in the range of ±10%, such as in the range of ±5%, of the alkali clean stability of SEQ ID NO:88 or of SEQ ID NO:89 after incubation in 0.5 M NaOH, such as after repeated incubation with 0.5 M NaOH.
149. A polynucleotide encoding a VH3 binding polypeptide according to any one of items 1-126, 138-140 and 143-148 or a VH3 binding polypeptide multimer according to any one of items 127-141 and 143-148.
150. Expression vector comprising a polynucleotide according to item 149.
151. Host cell comprising an expression vector according to item 150.
152. Method of producing a VH3 binding polypeptide according to any one of items 1-126, 138-140 and 143-148 or a VH3 binding polypeptide multimer according to any one of items 127-141 and 143-148, comprising
   culturing a host cell according to item 151 under conditions permissive of expression of said polypeptide or multimer from said expression vector, and
   isolating said polypeptide or multimer.
153. A separation matrix comprising a VH3 binding polypeptide according to any one of items 1-126, 138-140 and 143-148 or a VH3 binding polypeptide multimer according to any one of items 127-148 being coupled to a solid support.
154. Separation matrix according to item 153 wherein said VH3 binding polypeptide multimer is a multimer according to any one of items 127-148.
155. Separation matrix according to item 153 or 154 wherein said VH3 binding polypeptide multimer is a tetramer or a hexamer.
156. Separation matrix according to any one of items 153-155 wherein said VH3 binding polypeptide comprises a sequence selected from the group consisting of SEQ ID NO:1-87, such as the group consisting of SEQ ID NO:1-82; such as the group consisting of SEQ ID NO:1, 2, 4-43 and 45-82;
   such as the group consisting of SEQ ID NO:5-7, 9-11, 13-41, 46-48, 50-52 and 54-82; such as the group consisting of SEQ ID NO:6-7, 10, 13-41, 47-48, 51 and 54-82; such as the group consisting of SEQ ID NO:6-7, 10, 13, 15-41, 47-48, 51, 54 and 56-82; such as the group consisting of SEQ ID NO:6, 10, 13, 15-41, 47, 51, 54 and 56-82; such as the group consisting of SEQ ID NO:13, 15-41, 54 and 56-82; such as the group consisting of SEQ ID NO:13, 15-40, 54 and 56-81; such as the group consisting of SEQ ID NO:13, 15, 54 and 56.
157. Separation matrix according to any one of items 153-156 wherein said VH3 binding polypeptide comprises a sequence selected from the group consisting of SEQ ID NO:1-41; such as the group consisting of SEQ ID NO:1, 2, 4-41; such as the group consisting of SEQ ID NO:5-7, 9-11, 13-41; such as the group consisting of SEQ ID NO:6-7, 10, 13-41; such as the group consisting of SEQ ID NO:6-7, 10, 13, 15-41; such as the group consisting of SEQ ID NO:6, 10, 13, 15-41; such as the group consisting of SEQ ID NO:13, 15-41; such as the group consisting of SEQ ID NO:13, 15-40; such as the group consisting of SEQ ID NO:13 and 15.
158. Separation matrix according to any one of items 153-156 wherein said VH3 binding polypeptide comprises a sequence selected from the group consisting of SEQ ID NO:42-82; such as the group consisting of SEQ ID NO:42-43 and 45-82; such as the group consisting of SEQ ID NO:46-48, 50-52 and 54-82; such as the group consisting of SEQ ID NO:47-48, 51 and 54-82; such as the group consisting of SEQ ID NO:47-48, 51, 54 and 56-82; such as the group consisting of SEQ ID NO:47, 51, 54 and 56-82; such as the group consisting of SEQ ID NO:54 and 56-82; such as the group consisting of SEQ ID NO:54 and 56-81; such as the group consisting of SEQ ID NO:54 and 56.
159. Separation matrix according to any one of items 153-157 wherein said VH3 binding polypeptide comprises a sequence selected from the group consisting of SEQ ID NO:13, SEQ ID NO:14 or SEQ ID NO:15.
160. Separation matrix according to any one of items 153-156 and 158 wherein said VH3 binding polypeptide comprises a sequence selected from the group consisting of SEQ ID NO:54, SEQ ID NO:55 or SEQ ID NO:56.
161. Separation matrix according to any one of items 153-156, wherein said VH3 binding polypeptide comprises sequence SEQ ID NO:41 or SEQ ID NO:82
162. Separation matrix according to any one of items 153-157 and 159, comprising a VH3 binding multimer selected from the group consiting of SEQ ID NO:175, SEQ ID NO:176 and SEQ ID NO:177.
163. Separation matrix according to any one of items 153-162, wherein the solid support comprises a polymer selected from the group consisting of polyhydroxy polymer, such as a polysaccharide; polyvinyl alcohol, a polyhydroxyalkyl acrylate, a polyhydroxyalkyl methacrylate, a polyacrylamide, and a polymethacrylamide.
164. Separation matrix according to item 163 wherein said polymer is a polysaccharide, preferably a polysaccharide selected from dextran, starch, cellulose, pullulan, agar and agarose.
165. Separation matrix according to any one of items 153-164 wherein said solid support is in fibrous, beaded or particle form.
166. Separation matrix according to any one of items 153-165, wherein said VH3 binding polypeptide is coupled to the solid support by covalent or non-covalent binding.
167. A method of isolating an immunoglobulin or fragment thereof comprising
a) contacting a liquid sample comprising said immunoglobulin or a fragment thereof with a separation matrix according to any one of items 153-166.
168. Method according to item 167 further comprising
b) washing said separation matrix with a washing liquid, and
c) eluting the immunoglobulin or fragment thereof from the separation matrix with an elution liquid, and optionally
d) cleaning the separation matrix with a cleaning liquid.
169. Method according to any one of items 167-168, wherein said fragment is selected from Fab fragments, Fab′ fragments, F(ab′)₂, scFab fragments, Fv fragments, scFv fragments, diabodies, triabodies and minibodies.
170. Method according to any one of items 167-169, wherein said immunoglobulin or fragment thereof is an asymmetric immunoglobulin or fragment thereof.
171. Method according to items 170, wherein said asymmetric immunoglobulin or fragment thereof is an multispecific immunoglobulin or fragment thereof, such as at least bispecific or trispecific immunoglobulin or fragment thereof.
172. Method according to any one of items 167-171, wherein said separation matrix comprises a VH3 binding polypeptide according to SEQ ID NO:14 or a multimer comprising SEQ ID NO:14 and the pH-level at elution is above 4, such as approximately in the range of 4.10-4.20, such as approximately 4.15.
173. Method according to any one of items 167-171, wherein said separation matrix comprises a VH3 binding polypeptide according to SEQ ID NO:15 or a multimer comprising SEQ ID NO:15 and the pH-level at elution is below 4, such as approximately in the range of 3.7-3.80, such as approximately 3.75.
174. Method of isolating an asymmetric immunoglobulin or fragment thereof comprising the steps of:
a) providing a liquid sample comprising a mixture of at least one symmetric immunoglobulin or fragment thereof and at least one asymmetric immunoglobulin or fragment thereof;
b) contacting said liquid sample with a separation matrix to which said symmetric immunoglobulin or fragment thereof and said asymmetric immunoglobulin or fragment thereof bind with different avidity, wherein said separation matrix is defined according to claim 14;
c) eluting said immunoglobulins or fragments thereof from the separation matrix, whereby said symmetric immunoglobulin or fragment thereof and said asymmetric immunoglobulin or fragment thereof are at least partially eluted separately from each other, and optionally
d) cleaning the separation matrix with a cleaning liquid.
175. A method according to item 174, wherein step c) involves:
c) eluting said immunoglobulins or fragments thereof from the separation matrix in a pH-gradient or a stepwise elution with at least two different pH-levels, whereby said symmetric immunoglobulin or fragment thereof and said asymmetric immunoglobulin or fragment thereof are at least partially eluted at different pH-levels.
176. Method of isolating an asymmetric immunoglobulin or fragment thereof according to item 174 or 175, wherein said asymmetric immunoglobulin or fragment thereof comprises only one VH3 region and the symmetric immunoglobulin or fragment thereof comprises at least two VH3 regions or no VH3 regions.
177. Method of isolating an asymmetric immunoglobulin or fragment thereof according to any one of items 174-176, wherein said separation matrix is as defined in any one of items 153-166.
178. Method of isolating an asymmetric immunoglobulin or fragment thereof according to any one of items 174-177,
   wherein no more than 25%, such as no more than 20%, such as no more than 15%, such as no more than 10%, of the of the asymmetric immunoglobulin or fragment thereof is present in the same eluate as the symmetric immunoglobulin or fragment thereof.
179. Method of isolating an asymmetric immunoglobulin or fragment thereof according to any one of items 174-178, wherein said asymmetric immunoglobulin or fragment thereof is an multispecific immunoglobulin or fragment thereof, such as at least bispecific or trispecific immunoglobulin or fragment thereof.
180. VH3 binding polypeptide derived from a Staphylococcus Protein A (SpA) or any domain thereof, wherein the positions, which in an alignment with SEQ ID NO:89 corresponds to positions X₉, X₁₀, X₁₁, X₁₃, X₁₄, X₁₇, X₁₈ and X₂₆ in said SEQ ID NO:89, independently of each other are as follows:
X₉ is selected from Q, Y and A;
X₁₀ is selected from Q and Y;
X₁₁ is selected from T, E and R;
X₁₃ is selected from L, E, R, A and Q;
X₁₄ is selected from L, E, R, A, Q and W;
X₁₇ is selected from A, H and L;
X₁₈ is selected from R, L and H;
X₂₆ is selected from Q and S;
X₂₈ is selected from N and A; and
X₂₉ is selected from A and G.
181. VH3 binding polypeptide derived from a Staphylococcus Protein A (SpA) or any domain thereof, wherein the positions, which in an alignment with SEQ ID NO:88 corresponds to positions X₉, X₁₀, X₁₁, X₁₃, X₁₄, X₁₇, X₁₈ and X₂₆ in said SEQ ID NO:88, independently of each other are as follows:
X₉ is selected from Q, Y and A;
X₁₀ is selected from Q and Y;
X₁₁ is selected from T, E and R;
X₁₃ is selected from L, E, R, A and Q;
X₁₄ is selected from L, E, R, A, Q and W;
X₁₇ is selected from A, H and L;
X₁₈ is selected from R, L and H;
X₂₆ is selected from Q and S;
X₂₈ is selected from N and A; and
X₂₉ is selected from A and G.

1. VH3 binding polypeptide derived from a Staphylococcus Protein A (SpA) or any domain thereof, wherein said polypeptide has binding affinity for a VH3 region of trastuzumab, and has lower binding affinity for an Fc region of trastuzumab, compared to the binding affinity of SEQ ID NO:88 for the same Fc region and wherein said VH3 binding polypeptide is alkali clean stabile.

2. VH3 binding polypeptide according to claim 1 comprising a sequence A, which Sequence A consists of an amino acid sequence selected from i), ii) and iii),
wherein i), ii) and iii) are defined as follows:
i)
(SEQ ID NO: 95)
X₈X₉X₁₀X₁₁AX₁₃X₁₄X₁₅X₁₆X₁₇X₁₈X₁₉PNLX₂₃X₂₄X₂₅X₂₆RX₂₈X₂₉FIQS
LX₃₅X₃₆
wherein, independently from each other,
X₈ is selected from E, D and A;
X₁₅ is selected from E and Q;
X₁₆ is selected from I and V;
X₁₉ is selected from L and M;
X₂₃ is selected from N and T;
X₂₄ is selected from A and E, such as is E;
X₂₅ is selected from D and E, such as is E;
X₃₅ is selected from K, R and H;
X₃₆ is selected from D and H;
ii) an amino acid sequence which has at least 83% identity to a sequence defined by i)
iii) an amino acid sequence which has at least 70% identity to any sequence selected from the group consisting of: residues 8-36 in SEQ ID NO:88, residues 8-36 in SEQ ID NO:89, residues 8-36 in SEQ ID NO:90, residues 8-36 in SEQ ID NO:91, residues 8-36 in SEQ ID NO:92, residues 11-39 in SEQ ID NO:93 and residues 1-29 in SEQ ID NO:94,
wherein additionally, in each of i), ii) and iii) independently from each other,
X₉ is selected from Q, Y and A;
X₁₀ is selected from Q and Y;
X₁₁ is selected from T, E and R;
X₁₃ is selected from L, E, R, A and Q;
X₁₄ is selected from L, E, R, A, Q and W;
X₁₇ is selected from A, H and L;
X₁₈ is selected from R, L and H;
X₂₆ is selected from Q and S;
X₂₈ is selected from N and A; and
X₂₉ is selected from A and G.

3. VH3 binding polypeptide according to claim 2, wherein X₉ is A and X₁₁ is R.

4. VH3 binding polypeptide according to claim 2, wherein X₉X₁₀X₁₁X₁₃X₁₄ is selected from the group consisting of AQRLA and AYRLA; such as wherein X₉X₁₀X₁₁X₁₃X₁₄ is AYRLA

5. VH3 binding polypeptide according to claim 2, wherein X₁₇X₁₈ is AR.

6. VH3 binding polypeptide according to claim 2, wherein sequence i) corresponds to the sequence from position 8 to position 36 in a sequence selected from the group consisting of SEQ ID NO:1-82.

7. VH3 binding polypeptide according to claim 2, further comprising a Sequence B arranged C-terminally of said sequence A, which Sequence B consists of an amino acid sequence selected from iv) and v), and wherein iv) and v) are defined as follows:
iv)
(SEQ ID NO: 96)
DPSX₄₀SX₄₂X₄₃X₄₄LX₄₆EAX₄₉X₅₀LNX₅₃X₅₄
wherein, independently from each other,
X₄₀ is selected from Q, T and V;
X₄₂ is selected from A, K, L and T;
X₄₃ is selected from N, E, A, and S;
X₄₄ is selected from L, I and V;
X₄₆ is selected from A, G, S and K, such as from A, S and K;
X₄₉ is selected from K and Q, such as is K;
X₅₀ is selected from K and R;
X₅₃ is selected from D, E and K; and
X₅₄ is selected from A and S;
v) an amino acid sequence which has at least 75% identity to a sequence defined by iv).

8. VH3 binding polypeptide according to claim 2, comprising a binding module sequence C, which Sequence C consists of Sequence A according to claim 2 and Sequence B in the following order from the N-terminus to the C-terminus
[Sequence A]-[Sequence B]
or any amino acid sequence which has at least 70% identity to any sequence selected from the group consisting of: residues 8-54 in SEQ ID NO:88, residues 8-54 in SEQ ID NO:89, residues 8-54 in SEQ ID NO:90, residues 8-54 in SEQ ID NO:91, residues 8-54 in SEQ ID NO:92, residues 11-57 in SEQ ID NO:93 and residues 1-47 in SEQ ID NO:94.

9. VH3 binding polypeptide according to claim 2, comprising a binding module Sequence C selected from the group consisting of:
vi)
(SEQ ID NO: 113)
[Sequence A]-DPSQSANLLAEAKKLNDA;
wherein [Sequence A] is as defined in claim 2;
vii) an amino acid sequence which has at least 85% identity to the sequence defined in vi);
viii) [Sequence A]-DPSVSKAILAEAKKLNDA (SEQ ID NO:115);
viii)
(SEQ ID NO: 115)
[Sequence A]-DPSVSKAILAEAKKLNDA
wherein [Sequence A] is as defined in claim 2;
ix) an amino acid sequence which has at least 85% identity to the sequence defined in viii);
x)
(SEQ ID NO: 114)
[Sequence A]-DPSVSKEILAEAKKLNDA
wherein [Sequence A] is as defined in claim 2; and
xi) an amino acid sequence which has at least 85% identity to the sequence defined in x).

10. VH3 binding polypeptide according to claim 2, wherein sequence vi) or viii) corresponds to the sequence from position 8 to position 54 in a sequence selected from the group consisting of SEQ ID NO:1-82, such as the group consisting of SEQ ID NO:1-41, such as the group consisting of SEQ ID NO:1-40, the group consisting of SEQ ID NO:1-15, such as the group consisting of SEQ ID NO:13-15, such as SEQ ID NO:15.

11. VH3 binding polypeptide multimer, wherein each monomer of the multimer comprises a VH3 binding polypeptide which is independently selected from any VH3 binding polypeptide defined in claim 1 and wherein the multimer preferably is a tetramer or a hexamer.

12. VH3 binding polypeptide multimer according to claim 11, wherein said multimer comprises a sequence selected from the group consiting of SEQ ID NO:175, SEQ ID NO:176 and SEQ ID NO:177, such as wherein said sequence is SEQ ID NO:177.

13. VH3 binding polypeptide according to claim 1, which exhibits an alkali clean stability of at least 35%, such as at least 40%, such as at least 40%, such as at least 45%, such as at least 50%, such as at least 55%, such as at least 60%, such as at least 65%, such as at least 70%, such as at least 75%, such as at least 80%, such as at least 85%, such as at least 90%, such as at least 95% of the alkali clean stability of SEQ ID NO:88 after incubation in 0.5 M NaOH.

14. A separation matrix comprising a a VH3 binding polypeptide multimer according to claim 11, coupled to a solid support, which solid support preferably is in fibrous, beaded or particle form.

15. A method of isolating an immunoglobulin or fragment thereof comprising a) contacting a liquid sample comprising said immunoglobulin or fragment thereof with a separation matrix according to claim 14.

16. Method of isolating an asymmetric immunoglobulin or fragment thereof comprising the steps of:
a) providing a liquid sample comprising a mixture of at least one symmetric immunoglobulin or fragment thereof and at least one asymmetric immunoglobulin or fragment thereof;
b) contacting said liquid sample with a separation matrix to which said symmetric immunoglobulin or fragment thereof and said asymmetric immunoglobulin or fragment thereof bind with different avidity, wherein said separation matrix is defined according to claim 14;
c) eluting said immunoglobulins or fragments thereof from the separation matrix, whereby said symmetric immunoglobulin or fragment thereof and said asymmetric immunoglobulin or fragment thereof are at least partially eluted separately from each other, and optionally
d) cleaning the separation matrix with a cleaning liquid.


SEQUENCE LISTING (239 sequences)

SEQ ID NO: 1 - protein, 58 aa, synthetic construct
  REGION 1..58: note = Engineered VH3 binding polypeptide
VDAKFDKEQQTALAEILHLPNLTEEQRNAFIQSLKDDPSVSKAILAEAKKLNDAQAPK

SEQ ID NO: 2 - protein, 58 aa, synthetic construct
  REGION 1..58: note = Engineered VH3 binding polypeptide
VDAKFDKEQYTALAEILHLPNLTEEQRNAFIQSLKDDPSVSKAILAEAKKLNDAQAPK

SEQ ID NO: 3 - protein, 58 aa, synthetic construct
  REGION 1..58: note = Engineered VH3 binding polypeptide
VDAKFDKEYQTALAEILHLPNLTEEQRNAFIQSLKDDPSVSKAILAEAKKLNDAQAPK

SEQ ID NO: 4 - protein, 58 aa, synthetic construct
  REGION 1..58: note = Engineered VH3 binding polypeptide
VDAKFDKEQQTALAEIARLPNLTEEQRNAFIQSLKDDPSVSKAILAEAKKLNDAQAPK

SEQ ID NO: 5 - protein, 58 aa, synthetic construct
  REGION 1..58: note = Engineered VH3 binding polypeptide
VDAKFDKEAQEALAEILHLPNLTEEQRNAFIQSLKDDPSVSKAILAEAKKLNDAQAPK

SEQ ID NO: 6 - protein, 58 aa, synthetic construct
  REGION 1..58: note = Engineered VH3 binding polypeptide
VDAKFDKEAQRALAEILHLPNLTEEQRNAFIQSLKDDPSVSKAILAEAKKLNDAQAPK

SEQ ID NO: 7 - protein, 58 aa, synthetic construct
  REGION 1..58: note = Engineered VH3 binding polypeptide
VDAKFDKEAYRALAEILHLPNLTEEQRNAFIQSLKDDPSVSKAILAEAKKLNDAQAPK

SEQ ID NO: 8 - protein, 58 aa, synthetic construct
  REGION 1..58: note = Engineered VH3 binding polypeptide
VDAKFDKEAYRALWEILHLPNLTEEQRNAFIQSLKDDPSVSKAILAEAKKLNDAQAPK

SEQ ID NO: 9 - protein, 58 aa, synthetic construct
  REGION 1..58: note = Engineered VH3 binding polypeptide
VDAKFDKEAYRALWEIARLPNLTEEQRNAFIQSLKDDPSVSKAILAEAKKLNDAQAPK

SEQ ID NO: 10 - protein, 58 aa, synthetic construct
  REGION 1..58: note = Engineered VH3 binding polypeptide
VDAKFDKEAYRALAEIARLPNLTEEQRNAFIQSLKDDPSVSKAILAEAKKLNDAQAPK

SEQ ID NO: 11 - protein, 58 aa, synthetic construct
  REGION 1..58: note = Engineered VH3 binding polypeptide
VDAKFDKEAYRALAEIHLLPNLTEEQRNAFIQSLKDDPSVSKAILAEAKKLNDAQAPK

SEQ ID NO: 12 - protein, 58 aa, synthetic construct
  REGION 1..58: note = Engineered VH3 binding polypeptide
VDAKFDKEAYRALWEIHLLPNLTEEQRNAFIQSLKDDPSVSKAILAEAKKLNDAQAPK

SEQ ID NO: 13 - protein, 58 aa, synthetic construct
  REGION 1..58: note = Engineered VH3 binding polypeptide
VDAKFDKEAYRALAEIARLPNLTEEQRNGFIQSLKDDPSVSKAILAEAKKLNDAQAPK

SEQ ID NO: 14 - protein, 58 aa, synthetic construct
  REGION 1..58: note = Engineered VH3 binding polypeptide
VDAKFDKEAYRALAEIARLPNLTEEQRAAFIQSLKDDPSVSKAILAEAKKLNDAQAPK

SEQ ID NO: 15 - protein, 58 aa, synthetic construct
  REGION 1..58: note = Engineered VH3 binding polypeptide
VDAKFDKEAYRALAEIARLPNLTEEQRAGFIQSLKDDPSVSKAILAEAKKLNDAQAPK

SEQ ID NO: 16 - protein, 58 aa, synthetic construct
  REGION 1..58: note = Engineered VH3 binding polypeptide
VDAKFDKEAYRAAAEIARLPNLTEEQRAGFIQSLKDDPSVSKAILAEAKKLNDAQAPK

SEQ ID NO: 17 - protein, 58 aa, synthetic construct
  REGION 1..58: note = Engineered VH3 binding polypeptide
VDAKFDKEAYRAAEEIARLPNLTEEQRAGFIQSLKDDPSVSKAILAEAKKLNDAQAPK

SEQ ID NO: 18 - protein, 58 aa, synthetic construct
  REGION 1..58: note = Engineered VH3 binding polypeptide
VDAKFDKEAYRAALEIARLPNLTEEQRAGFIQSLKDDPSVSKAILAEAKKLNDAQAPK

SEQ ID NO: 19 - protein, 58 aa, synthetic construct
  REGION 1..58: note = Engineered VH3 binding polypeptide
VDAKFDKEAYRAAQEIARLPNLTEEQRAGFIQSLKDDPSVSKAILAEAKKLNDAQAPK

SEQ ID NO: 20 - protein, 58 aa, synthetic construct
  REGION 1..58: note = Engineered VH3 binding polypeptide
VDAKFDKEAYRAAREIARLPNLTEEQRAGFIQSLKDDPSVSKAILAEAKKLNDAQAPK

SEQ ID NO: 21 - protein, 58 aa, synthetic construct
  REGION 1..58: note = Engineered VH3 binding polypeptide
VDAKFDKEAYRAEAEIARLPNLTEEQRAGFIQSLKDDPSVSKAILAEAKKLNDAQAPK

SEQ ID NO: 22 - protein, 58 aa, synthetic construct
  REGION 1..58: note = Engineered VH3 binding polypeptide
VDAKFDKEAYRAEEEIARLPNLTEEQRAGFIQSLKDDPSVSKAILAEAKKLNDAQAPK

SEQ ID NO: 23 - protein, 58 aa, synthetic construct
  REGION 1..58: note = Engineered VH3 binding polypeptide
VDAKFDKEAYRAELEIARLPNLTEEQRAGFIQSLKDDPSVSKAILAEAKKLNDAQAPK

SEQ ID NO: 24 - protein, 58 aa, synthetic construct
  REGION 1..58: note = Engineered VH3 binding polypeptide
VDAKFDKEAYRAEQEIARLPNLTEEQRAGFIQSLKDDPSVSKAILAEAKKLNDAQAPK

SEQ ID NO: 25 - protein, 58 aa, synthetic construct
  REGION 1..58: note = Engineered VH3 binding polypeptide
VDAKFDKEAYRAEREIARLPNLTEEQRAGFIQSLKDDPSVSKAILAEAKKLNDAQAPK

SEQ ID NO: 26 - protein, 58 aa, synthetic construct; identical to SEQ ID NO: 15
  REGION 1..58: note = Engineered VH3 binding polypeptide

SEQ ID NO: 27 - protein, 58 aa, synthetic construct
  REGION 1..58: note = Engineered VH3 binding polypeptide
VDAKFDKEAYRALEEIARLPNLTEEQRAGFIQSLKDDPSVSKAILAEAKKLNDAQAPK

SEQ ID NO: 28 - protein, 58 aa, synthetic construct
  REGION 1..58: note = Engineered VH3 binding polypeptide
VDAKFDKEAYRALLEIARLPNLTEEQRAGFIQSLKDDPSVSKAILAEAKKLNDAQAPK

SEQ ID NO: 29 - protein, 58 aa, synthetic construct
  REGION 1..58: note = Engineered VH3 binding polypeptide
VDAKFDKEAYRALQEIARLPNLTEEQRAGFIQSLKDDPSVSKAILAEAKKLNDAQAPK

SEQ ID NO: 30 - protein, 58 aa, synthetic construct
  REGION 1..58: note = Engineered VH3 binding polypeptide
VDAKFDKEAYRALREIARLPNLTEEQRAGFIQSLKDDPSVSKAILAEAKKLNDAQAPK

SEQ ID NO: 31 - protein, 58 aa, synthetic construct
  REGION 1..58: note = Engineered VH3 binding polypeptide
VDAKFDKEAYRAQAEIARLPNLTEEQRAGFIQSLKDDPSVSKAILAEAKKLNDAQAPK

SEQ ID NO: 32 - protein, 58 aa, synthetic construct
  REGION 1..58: note = Engineered VH3 binding polypeptide
VDAKFDKEAYRAQEEIARLPNLTEEQRAGFIQSLKDDPSVSKAILAEAKKLNDAQAPK

SEQ ID NO: 33 - protein, 58 aa, synthetic construct
  REGION 1..58: note = Engineered VH3 binding polypeptide
VDAKFDKEAYRAQLEIARLPNLTEEQRAGFIQSLKDDPSVSKAILAEAKKLNDAQAPK

SEQ ID NO: 34 - protein, 58 aa, synthetic construct
  REGION 1..58: note = Engineered VH3 binding polypeptide
VDAKFDKEAYRAQQEIARLPNLTEEQRAGFIQSLKDDPSVSKAILAEAKKLNDAQAPK

SEQ ID NO: 35 - protein, 58 aa, synthetic construct
  REGION 1..58: note = Engineered VH3 binding polypeptide
VDAKFDKEAYRAQREIARLPNLTEEQRAGFIQSLKDDPSVSKAILAEAKKLNDAQAPK

SEQ ID NO: 36 - protein, 58 aa, synthetic construct
  REGION 1..58: note = Engineered VH3 binding polypeptide
VDAKFDKEAYRARAEIARLPNLTEEQRAGFIQSLKDDPSVSKAILAEAKKLNDAQAPK

SEQ ID NO: 37 - protein, 58 aa, synthetic construct
  REGION 1..58: note = Engineered VH3 binding polypeptide
VDAKFDKEAYRAREEIARLPNLTEEQRAGFIQSLKDDPSVSKAILAEAKKLNDAQAPK

SEQ ID NO: 38 - protein, 58 aa, synthetic construct
  REGION 1..58: note = Engineered VH3 binding polypeptide
VDAKFDKEAYRARLEIARLPNLTEEQRAGFIQSLKDDPSVSKAILAEAKKLNDAQAPK

SEQ ID NO: 39 - protein, 58 aa, synthetic construct
  REGION 1..58: note = Engineered VH3 binding polypeptide
VDAKFDKEAYRARQEIARLPNLTEEQRAGFIQSLKDDPSVSKAILAEAKKLNDAQAPK

SEQ ID NO: 40 - protein, 58 aa, synthetic construct
  REGION 1..58: note = Engineered VH3 binding polypeptide
VDAKFDKEAYRARREIARLPNLTEEQRAGFIQSLKDDPSVSKAILAEAKKLNDAQAPK

SEQ ID NO: 41 - protein, 58 aa, synthetic construct
  REGION 1..58: note = Engineered VH3 binding polypeptide
VDAKFDKEAYRALAEIARLPNLTEESRAGFIQSLKDDPSVSKAILAEAKKLNDAQAPK

SEQ ID NO: 42 - protein, 58 aa, synthetic construct
  REGION 1..58: note = Engineered VH3 binding polypeptide
VDNKFNKEQQTALAEILHLPNLNEEQRNAFIQSLKDDPSQSANLLAEAKKLNDAQAPK

SEQ ID NO: 43 - protein, 58 aa, synthetic construct
  REGION 1..58: note = Engineered VH3 binding polypeptide
VDNKFNKEQYTALAEILHLPNLNEEQRNAFIQSLKDDPSQSANLLAEAKKLNDAQAPK

SEQ ID NO: 44 - protein, 58 aa, synthetic construct
  REGION 1..58: note = Engineered VH3 binding polypeptide
VDNKFNKEYQTALAEILHLPNLNEEQRNAFIQSLKDDPSQSANLLAEAKKLNDAQAPK

SEQ ID NO: 45 - protein, 58 aa, synthetic construct
  REGION 1..58: note = Engineered VH3 binding polypeptide
VDNKFNKEQQTALAEIARLPNLNEEQRNAFIQSLKDDPSQSANLLAEAKKLNDAQAPK

SEQ ID NO: 46 - protein, 58 aa, synthetic construct
  REGION 1..58: note = Engineered VH3 binding polypeptide
VDNKFNKEAQEALAEILHLPNLNEEQRNAFIQSLKDDPSQSANLLAEAKKLNDAQAPK

SEQ ID NO: 47 - protein, 58 aa, synthetic construct
  REGION 1..58: note = Engineered VH3 binding polypeptide
VDNKFNKEAQRALAEILHLPNLNEEQRNAFIQSLKDDPSQSANLLAEAKKLNDAQAPK

SEQ ID NO: 48 - protein, 58 aa, synthetic construct
  REGION 1..58: note = Engineered VH3 binding polypeptide
VDNKFNKEAYRALAEILHLPNLNEEQRNAFIQSLKDDPSQSANLLAEAKKLNDAQAPK

SEQ ID NO: 49 - protein, 58 aa, synthetic construct
  REGION 1..58: note = Engineered VH3 binding polypeptide
VDNKFNKEAYRALWEILHLPNLNEEQRNAFIQSLKDDPSQSANLLAEAKKLNDAQAPK

SEQ ID NO: 50 - protein, 58 aa, synthetic construct
  REGION 1..58: note = Engineered VH3 binding polypeptide
VDNKFNKEAYRALWEIARLPNLNEEQRNAFIQSLKDDPSQSANLLAEAKKLNDAQAPK

SEQ ID NO: 51 - protein, 58 aa, synthetic construct
  REGION 1..58: note = Engineered VH3 binding polypeptide
VDNKFNKEAYRALAEIARLPNLNEEQRNAFIQSLKDDPSQSANLLAEAKKLNDAQAPK

SEQ ID NO: 52 - protein, 58 aa, synthetic construct
  REGION 1..58: note = Engineered VH3 binding polypeptide
VDNKFNKEAYRALAEIHLLPNLNEEQRNAFIQSLKDDPSQSANLLAEAKKLNDAQAPK

SEQ ID NO: 53 - protein, 58 aa, synthetic construct
  REGION 1..58: note = Engineered VH3 binding polypeptide
VDNKFNKEAYRALWEIHLLPNLNEEQRNAFIQSLKDDPSQSANLLAEAKKLNDAQAPK

SEQ ID NO: 54 - protein, 58 aa, synthetic construct
  REGION 1..58: note = Engineered VH3 binding polypeptide
VDNKFNKEAYRALAEIARLPNLNEEQRNGFIQSLKDDPSQSANLLAEAKKLNDAQAPK

SEQ ID NO: 55 - protein, 58 aa, synthetic construct
  REGION 1..58: note = Engineered VH3 binding polypeptide
VDNKFNKEAYRALAEIARLPNLNEEQRAAFIQSLKDDPSQSANLLAEAKKLNDAQAPK

SEQ ID NO: 56 - protein, 58 aa, synthetic construct
  REGION 1..58: note = Engineered VH3 binding polypeptide
VDNKFNKEAYRALAEIARLPNLNEEQRAGFIQSLKDDPSQSANLLAEAKKLNDAQAPK

SEQ ID NO: 57 - protein, 58 aa, synthetic construct
  REGION 1..58: note = Engineered VH3 binding polypeptide
VDNKFNKEAYRAAAEIARLPNLNEEQRAGFIQSLKDDPSQSANLLAEAKKLNDAQAPK

SEQ ID NO: 58 - protein, 58 aa, synthetic construct
  REGION 1..58: note = Engineered VH3 binding polypeptide
VDNKFNKEAYRAAEEIARLPNLNEEQRAGFIQSLKDDPSQSANLLAEAKKLNDAQAPK

SEQ ID NO: 59 - protein, 58 aa, synthetic construct
  REGION 1..58: note = Engineered VH3 binding polypeptide
VDNKFNKEAYRAALEIARLPNLNEEQRAGFIQSLKDDPSQSANLLAEAKKLNDAQAPK

SEQ ID NO: 60 - protein, 58 aa, synthetic construct
  REGION 1..58: note = Engineered VH3 binding polypeptide
VDNKFNKEAYRAAQEIARLPNLNEEQRAGFIQSLKDDPSQSANLLAEAKKLNDAQAPK

SEQ ID NO: 61 - protein, 58 aa, synthetic construct
  REGION 1..58: note = Engineered VH3 binding polypeptide
VDNKFNKEAYRAAREIARLPNLNEEQRAGFIQSLKDDPSQSANLLAEAKKLNDAQAPK

SEQ ID NO: 62 - protein, 58 aa, synthetic construct
  REGION 1..58: note = Engineered VH3 binding polypeptide
VDNKFNKEAYRAEAEIARLPNLNEEQRAGFIQSLKDDPSQSANLLAEAKKLNDAQAPK

SEQ ID NO: 63 - protein, 58 aa, synthetic construct
  REGION 1..58: note = Engineered VH3 binding polypeptide
VDNKFNKEAYRAEEEIARLPNLNEEQRAGFIQSLKDDPSQSANLLAEAKKLNDAQAPK

SEQ ID NO: 64 - protein, 58 aa, synthetic construct
  REGION 1..58: note = Engineered VH3 binding polypeptide
VDNKFNKEAYRAELEIARLPNLNEEQRAGFIQSLKDDPSQSANLLAEAKKLNDAQAPK

SEQ ID NO: 65 - protein, 58 aa, synthetic construct
  REGION 1..58: note = Engineered VH3 binding polypeptide
VDNKFNKEAYRAEQEIARLPNLNEEQRAGFIQSLKDDPSQSANLLAEAKKLNDAQAPK

SEQ ID NO: 66 - protein, 58 aa, synthetic construct
  REGION 1..58: note = Engineered VH3 binding polypeptide
VDNKFNKEAYRAEREIARLPNLNEEQRAGFIQSLKDDPSQSANLLAEAKKLNDAQAPK

SEQ ID NO: 67 - protein, 58 aa, synthetic construct; identical to SEQ ID NO: 56
  REGION 1..58: note = Engineered VH3 binding polypeptide

SEQ ID NO: 68 - protein, 58 aa, synthetic construct
  REGION 1..58: note = Engineered VH3 binding polypeptide
VDNKFNKEAYRALEEIARLPNLNEEQRAGFIQSLKDDPSQSANLLAEAKKLNDAQAPK

SEQ ID NO: 69 - protein, 58 aa, synthetic construct
  REGION 1..58: note = Engineered VH3 binding polypeptide
VDNKFNKEAYRALLEIARLPNLNEEQRAGFIQSLKDDPSQSANLLAEAKKLNDAQAPK

SEQ ID NO: 70 - protein, 58 aa, synthetic construct
  REGION 1..58: note = Engineered VH3 binding polypeptide
VDNKFNKEAYRALQEIARLPNLNEEQRAGFIQSLKDDPSQSANLLAEAKKLNDAQAPK

SEQ ID NO: 71 - protein, 58 aa, synthetic construct
  REGION 1..58: note = Engineered VH3 binding polypeptide
VDNKFNKEAYRALREIARLPNLNEEQRAGFIQSLKDDPSQSANLLAEAKKLNDAQAPK

SEQ ID NO: 72 - protein, 58 aa, synthetic construct
  REGION 1..58: note = Engineered VH3 binding polypeptide
VDNKFNKEAYRAQAEIARLPNLNEEQRAGFIQSLKDDPSQSANLLAEAKKLNDAQAPK

SEQ ID NO: 73 - protein, 58 aa, synthetic construct
  REGION 1..58: note = Engineered VH3 binding polypeptide
VDNKFNKEAYRAQEEIARLPNLNEEQRAGFIQSLKDDPSQSANLLAEAKKLNDAQAPK

SEQ ID NO: 74 - protein, 58 aa, synthetic construct
  REGION 1..58: note = Engineered VH3 binding polypeptide
VDNKFNKEAYRAQLEIARLPNLNEEQRAGFIQSLKDDPSQSANLLAEAKKLNDAQAPK

SEQ ID NO: 75 - protein, 58 aa, synthetic construct
  REGION 1..58: note = Engineered VH3 binding polypeptide
VDNKFNKEAYRAQQEIARLPNLNEEQRAGFIQSLKDDPSQSANLLAEAKKLNDAQAPK

SEQ ID NO: 76 - protein, 58 aa, synthetic construct
  REGION 1..58: note = Engineered VH3 binding polypeptide
VDNKFNKEAYRAQREIARLPNLNEEQRAGFIQSLKDDPSQSANLLAEAKKLNDAQAPK

SEQ ID NO: 77 - protein, 58 aa, synthetic construct
  REGION 1..58: note = Engineered VH3 binding polypeptide
VDNKFNKEAYRARAEIARLPNLNEEQRAGFIQSLKDDPSQSANLLAEAKKLNDAQAPK

SEQ ID NO: 78 - protein, 58 aa, synthetic construct
  REGION 1..58: note = Engineered VH3 binding polypeptide
VDNKFNKEAYRAREEIARLPNLNEEQRAGFIQSLKDDPSQSANLLAEAKKLNDAQAPK

SEQ ID NO: 79 - protein, 58 aa, synthetic construct
  REGION 1..58: note = Engineered VH3 binding polypeptide
VDNKFNKEAYRARLEIARLPNLNEEQRAGFIQSLKDDPSQSANLLAEAKKLNDAQAPK

SEQ ID NO: 80 - protein, 58 aa, synthetic construct
  REGION 1..58: note = Engineered VH3 binding polypeptide
VDNKFNKEAYRARQEIARLPNLNEEQRAGFIQSLKDDPSQSANLLAEAKKLNDAQAPK

SEQ ID NO: 81 - protein, 58 aa, synthetic construct
  REGION 1..58: note = Engineered VH3 binding polypeptide
VDNKFNKEAYRARREIARLPNLNEEQRAGFIQSLKDDPSQSANLLAEAKKLNDAQAPK

SEQ ID NO: 82 - protein, 58 aa, synthetic construct
  REGION 1..58: note = Engineered VH3 binding polypeptide
VDNKFNKEAYRALAEIARLPNLNEESRAGFIQSLKDDPSQSKAILAEAKKLNDAQAPK

SEQ ID NO: 83 - protein, 58 aa, synthetic construct
  REGION 1..58: note = Engineered VH3 binding polypeptide
ADNKFNKEAYRALAEIARLPNLTEEQRAGFIQSLKDDPSVSKEILAEAKKLNDAQAPK

SEQ ID NO: 84 - protein, 58 aa, synthetic construct
  REGION 1..58: note = Engineered VH3 binding polypeptide
ADNKFNKEAYRALAEIARLPNLNEEQRAGFIQSLKDDPSQSANLLAEAKKLNDAQAPK

SEQ ID NO: 85 - protein, 58 aa, synthetic construct
  REGION 1..58: note = Engineered VH3 binding polypeptide
ADNNFNKEAYRALAEIARMPNLNEEQRAGFIQSLKDDPSQSANLLSEAKKLNESQAPK

SEQ ID NO: 86 - protein, 61 aa, synthetic construct
  REGION 1..61: note = Engineered VH3 binding polypeptide
ADAQQNKFNKDAYRALAEIARMPNLNEEQRAGFIQSLKDDPSQSTNVLGEAKKLNESQAPK

SEQ ID NO: 87 - protein, 51 aa, synthetic construct
  REGION 1..51: note = Engineered VH3 binding polypeptide
AAYRALAQVARMPNLNADQRAGFIQSLKDDPSQSANVLGEAQKLNDSQAPK

SEQ ID NO: 88 - protein, 58 aa, synthetic construct
  REGION 1..58: note = Engineered VH3 binding polypeptide
VDAKFDKEAQEAFYEILHLPNLTEEQRNAFIQSLKDDPSVSKAILAEAKKLNDAQAPK

SEQ ID NO: 89 - protein, 58 aa, synthetic construct [sequence omitted]

SEQ ID NO: 90 - protein, 58 aa, Staphylococcus aureus
ADNKFNKEQQNAFYEILHLPNLTEEQRNGFIQSLKDDPSVSKEILAEAKKLNDAQAPK

SEQ ID NO: 91 - protein, 58 aa, Staphylococcus aureus [sequence omitted]

SEQ ID NO: 92 - protein, 58 aa, Staphylococcus aureus
ADNNFNKEQQNAFYEILNMPNLNEEQRNGFIQSLKDDPSQSANLLSEAKKLNESQAPK

SEQ ID NO: 93 - protein, 61 aa, Staphylococcus aureus
ADAQQNKFNKDQQSAFYEILNMPNLNEEQRNGFIQSLKDDPSQSTNVLGEAKKLNESQAPK

SEQ ID NO: 94 - protein, 51 aa, Staphylococcus aureus
AQQNAFYQVLNMPNLNADQRNGFIQSLKDDPSQSANVLGEAQKLNDSQAPK

SEQ ID NO: 95 - protein, 29 aa, synthetic construct
  REGION 1..29: note = Engineered VH3 binding polypeptide
  VARIANT 1..4: note = misc_feature - Xaa can be any naturally occurring amino acid
  VARIANT 6..12: note = misc_feature - Xaa can be any naturally occurring amino acid
  VARIANT 16..19: note = misc_feature - Xaa can be any naturally occurring amino acid
  VARIANT 21..22: note = misc_feature - Xaa can be any naturally occurring amino acid
  VARIANT 26: note = misc_feature - Xaa can be any naturally occurring amino acid
  VARIANT 28..29: note = misc_feature - Xaa can be any naturally occurring amino acid
XXXXAXXXXXXXPNLXXXXRXXFIQXLXX

SEQ ID NO: 96 - protein, 18 aa, synthetic construct
  REGION 1..18: note = Engineered VH3 binding polypeptide
  VARIANT 4: note = misc_feature - Xaa can be any naturally occurring amino acid
  VARIANT 6..8: note = misc_feature - Xaa can be any naturally occurring amino acid
  VARIANT 10: note = misc_feature - Xaa can be any naturally occurring amino acid
  VARIANT 13..14: note = misc_feature - Xaa can be any naturally occurring amino acid
  VARIANT 17..18: note = misc_feature - Xaa can be any naturally occurring amino acid
DPSXSXXXLXEAXXLNXX

SEQ ID NO: 97 - protein, 4 aa, synthetic construct
  REGION 1..4: note = Engineered VH3 binding polypeptide
DPSV

SEQ ID NO: 98 - protein, 4 aa, synthetic construct
  REGION 1..4: note = Engineered VH3 binding polypeptide
DPSQ

SEQ ID NO: 99 - protein, 4 aa, synthetic construct
  REGION 1..4: note = Engineered VH3 binding polypeptide
EPSQ

SEQ ID NO: 100 - protein, 4 aa, synthetic construct
  REGION 1..4: note = Engineered VH3 binding polypeptide
DPST

SEQ ID NO: 101 - protein, 4 aa, synthetic construct
  REGION 1..4: note = Engineered VH3 binding polypeptide
EPSV

SEQ ID NO: 102 - protein, 4 aa, synthetic construct
  REGION 1..4: note = Engineered VH3 binding polypeptide
DPSL

SEQ ID NO: 103 - protein, 14 aa, synthetic construct
  REGION 1..14: note = Engineered VH3 binding polypeptide
SANLLAEAKKLNDA

SEQ ID NO: 104 - protein, 14 aa, synthetic construct
  REGION 1..14: note = Engineered VH3 binding polypeptide
SKEILAEAKKLNDA

SEQ ID NO: 105 - protein, 14 aa, synthetic construct
  REGION 1..14: note = Engineered VH3 binding polypeptide
SKAILAEAKKLNDA

SEQ ID NO: 106 - protein, 14 aa, synthetic construct
  REGION 1..14: note = Engineered VH3 binding polypeptide
SANLLAEAKKLNDA

SEQ ID NO: 107 - protein, 14 aa, synthetic construct
  REGION 1..14: note = Engineered VH3 binding polypeptide
SANLLAEAKKLNES

SEQ ID NO: 108 - protein, 14 aa, synthetic construct
  REGION 1..14: note = Engineered VH3 binding polypeptide
STNVLGEAKKLNES

SEQ ID NO: 109 - protein, 13 aa, synthetic construct
  REGION 1..13: note = Engineered VH3 binding polypeptide
SANVLGEAQKLND

SEQ ID NO: 110 - protein, 14 aa, synthetic construct
  REGION 1..14: note = Engineered VH3 binding polypeptide
SLEILCEAKKLNDA

SEQ ID NO: 111 - protein, 14 aa, synthetic construct
  REGION 1..14: note = Engineered VH3 binding polypeptide
SLEILAEAKKLNDA

SEQ ID NO: 112 - protein, 14 aa, synthetic construct
  REGION 1..14: note = Engineered VH3 binding polypeptide
SKKILKEAKKLNKA

SEQ ID NO: 113 - protein, 47 aa, synthetic construct
  REGION 1..47: note = Engineered VH3 binding polypeptide
  VARIANT 1..4: note = misc_feature - Xaa can be any naturally occurring amino acid
  VARIANT 6..12: note = misc_feature - Xaa can be any naturally occurring amino acid
  VARIANT 16..19: note = misc_feature - Xaa can be any naturally occurring amino acid
  VARIANT 21..22: note = misc_feature - Xaa can be any naturally occurring amino acid
  VARIANT 26: note = misc_feature - Xaa can be any naturally occurring amino acid
  VARIANT 28..29: note = misc_feature - Xaa can be any naturally occurring amino acid
XXXXAXXXXXXXPNLXXXXRXXFIQXLXXDPSQSANLLAEAKKLNDA

SEQ ID NO: 114 - protein, 47 aa, synthetic construct
  REGION 1..47: note = Engineered VH3 binding polypeptide
  VARIANT 1..4: note = misc_feature - Xaa can be any naturally occurring amino acid
  VARIANT 6..12: note = misc_feature - Xaa can be any naturally occurring amino acid
  VARIANT 16..19: note = misc_feature - Xaa can be any naturally occurring amino acid
  VARIANT 21..22: note = misc_feature - Xaa can be any naturally occurring amino acid
  VARIANT 26: note = misc_feature - Xaa can be any naturally occurring amino acid
  VARIANT 28..29: note = misc_feature - Xaa can be any naturally occurring amino acid
XXXXAXXXXXXXPNLXXXXRXXFIQXLXXDPSVSKEILAEAKKLNDA

SEQ ID NO: 115 - protein, 47 aa, synthetic construct
  REGION 1..47: note = Engineered VH3 binding polypeptide
  VARIANT 1..4: note = misc_feature - Xaa can be any naturally occurring amino acid
  VARIANT 6..12: note = misc_feature - Xaa can be any naturally occurring amino acid
  VARIANT 16..19: note = misc_feature - Xaa can be any naturally occurring amino acid
  VARIANT 21..22: note = misc_feature - Xaa can be any naturally occurring amino acid
  VARIANT 26: note = misc_feature - Xaa can be any naturally occurring amino acid
  VARIANT 28..29: note = misc_feature - Xaa can be any naturally occurring amino acid
XXXXAXXXXXXXPNLXXXXRXXFIQXLXXDPSVSKAILAEAKKLNDA

SEQ ID NO: 116 - protein, 47 aa, synthetic construct
  REGION 1..47: note = Engineered VH3 binding polypeptide
  VARIANT 1..4: note = misc_feature - Xaa can be any naturally occurring amino acid
  VARIANT 6..12: note = misc_feature - Xaa can be any naturally occurring amino acid
  VARIANT 16..19: note = misc_feature - Xaa can be any naturally occurring amino acid
  VARIANT 21..22: note = misc_feature - Xaa can be any naturally occurring amino acid
  VARIANT 26: note = misc_feature - Xaa can be any naturally occurring amino acid
  VARIANT 28..29: note = misc_feature - Xaa can be any naturally occurring amino acid
XXXXAXXXXXXXPNLXXXXRXXFIQXLXXDPSQSANLLAEAKKLNES

SEQ ID NO: 117 - protein, 47 aa, synthetic construct
  REGION 1..47: note = Engineered VH3 binding polypeptide
  VARIANT 1..4: note = misc_feature - Xaa can be any naturally occurring amino acid
  VARIANT 6..12: note = misc_feature - Xaa can be any naturally occurring amino acid
  VARIANT 16..19: note = misc_feature - Xaa can be any naturally occurring amino acid
  VARIANT 21..22: note = misc_feature - Xaa can be any naturally occurring amino acid
  VARIANT 26: note = misc_feature - Xaa can be any naturally occurring amino acid
  VARIANT 28..29: note = misc_feature - Xaa can be any naturally occurring amino acid
XXXXAXXXXXXXPNLXXXXRXXFIQXLXXDPSVSLEILGEAKKLNDA

SEQ ID NO: 118 - protein, 47 aa, synthetic construct
  REGION 1..47: note = Engineered VH3 binding polypeptide
  VARIANT 1..4: note = misc_feature - Xaa can be any naturally occurring amino acid
  VARIANT 6..12: note = misc_feature - Xaa can be any naturally occurring amino acid
  VARIANT 16..19: note = misc_feature - Xaa can be any naturally occurring amino acid
  VARIANT 21..22: note = misc_feature - Xaa can be any naturally occurring amino acid
  VARIANT 26: note = misc_feature - Xaa can be any naturally occurring amino acid
  VARIANT 28..29: note = misc_feature - Xaa can be any naturally occurring amino acid
XXXXAXXXXXXXPNLXXXXRXXFIQXLXXDPSVSLEILCEAKKLNDA

SEQ ID NO: 119 - protein, 47 aa, synthetic construct
  REGION 1..47: note = Engineered VH3 binding polypeptide
  VARIANT 1..4: note = misc_feature - Xaa can be any naturally occurring amino acid
  VARIANT 6..12: note = misc_feature - Xaa can be any naturally occurring amino acid
  VARIANT 16..19: note = misc_feature - Xaa can be any naturally occurring amino acid
  VARIANT 21..22: note = misc_feature - Xaa can be any naturally occurring amino acid
  VARIANT 26: note = misc_feature - Xaa can be any naturally occurring amino acid
  VARIANT 28..29: note = misc_feature - Xaa can be any naturally occurring amino acid
XXXXAXXXXXXXPNLXXXXRXXFIQXLXXDPSVSLEILAEAKKLNDA

SEQ ID NO: 120 - protein, 47 aa, synthetic construct
  REGION 1..47: note = Engineered VH3 binding polypeptide
  VARIANT 1..4: note = misc_feature - Xaa can be any naturally occurring amino acid
  VARIANT 6..12: note = misc_feature - Xaa can be any naturally occurring amino acid
  VARIANT 16..19: note = misc_feature - Xaa can be any naturally occurring amino acid
  VARIANT 21..22: note = misc_feature - Xaa can be any naturally occurring amino acid
  VARIANT 26: note = misc_feature - Xaa can be any naturally occurring amino acid
  VARIANT 28..29: note = misc_feature - Xaa can be any naturally occurring amino acid
XXXXAXXXXXXXPNLXXXXRXXFIQXLXXDPSVSLALLAEAKKLNDA

SEQ ID NO: 121 - protein, 47 aa, synthetic construct
  REGION 1..47: note = Engineered VH3 binding polypeptide
  VARIANT 1..4: note = misc_feature - Xaa can be any naturally occurring amino acid
  VARIANT 6..12: note = misc_feature - Xaa can be any naturally occurring amino acid
  VARIANT 16..19: note = misc_feature - Xaa can be any naturally occurring amino acid
  VARIANT 21..22: note = misc_feature - Xaa can be any naturally occurring amino acid
  VARIANT 26: note = misc_feature - Xaa can be any naturally occurring amino acid
  VARIANT 28..29: note = misc_feature - Xaa can be any naturally occurring amino acid
XXXXAXXXXXXXPNLXXXXRXXFIQXLXXDPSQSANLLSEAKKLNES

SEQ ID NO: 122 - protein, 47 aa, synthetic construct
  REGION 1..47: note = Engineered VH3 binding polypeptide
  VARIANT 1..4: note = misc_feature - Xaa can be any naturally occurring amino acid
  VARIANT 6..12: note = misc_feature - Xaa can be any naturally occurring amino acid
  VARIANT 16..19: note = misc_feature - Xaa can be any naturally occurring amino acid
  VARIANT 21..22: note = misc_feature - Xaa can be any naturally occurring amino acid
  VARIANT 26: note = misc_feature - Xaa can be any naturally occurring amino acid
  VARIANT 28..29: note = misc_feature - Xaa can be any naturally occurring amino acid
XXXXAXXXXXXXPNLXXXXRXXFIQXLXXDPSQSTNVLGEAKKLNES

SEQ ID NO: 123 - protein, 47 aa, synthetic construct
  REGION 1..47: note = Engineered VH3 binding polypeptide
  VARIANT 1..4: note = misc_feature - Xaa can be any naturally occurring amino acid
  VARIANT 6..12: note = misc_feature - Xaa can be any naturally occurring amino acid
  VARIANT 16..19: note = misc_feature - Xaa can be any naturally occurring amino acid
  VARIANT 21..22: note = misc_feature - Xaa can be any naturally occurring amino acid
  VARIANT 26: note = misc_feature - Xaa can be any naturally occurring amino acid
  VARIANT 28..29: note = misc_feature - Xaa can be any naturally occurring amino acid
XXXXAXXXXXXXPNLXXXXRXXFIQXLXXDPSQSANVLGEAQKLNDS

SEQ ID NO: 124 - protein, 7 aa, synthetic construct
  REGION 1..7: note = Engineered VH3 binding polypeptide
ADNKFNK

SEQ ID NO: 125 - protein, 7 aa, synthetic construct
  REGION 1..7: note = Engineered VH3 binding polypeptide
VDAKFDK

SEQ ID NO: 126 - protein, 7 aa, synthetic construct
  REGION 1..7: note = Engineered VH3 binding polypeptide
VDNKFNK

SEQ ID NO: 127 - protein, 10 aa, synthetic construct
  REGION 1..10: note = Engineered VH3 binding polypeptide
ADAQQNKFNK

SEQ ID NO: 128 - protein, 7 aa, synthetic construct
  REGION 1..7: note = Engineered VH3 binding polypeptide
IAAKHDK

SEQ ID NO: 129 - protein, 7 aa, synthetic construct
  REGION 1..7: note = Engineered VH3 binding polypeptide
IAAQHDK

SEQ ID NO: 130 - protein, 7 aa, synthetic construct
  REGION 1..7: note = Engineered VH3 binding polypeptide
ADNNFNK

SEQ ID NO: 131 - protein, 7 aa, synthetic construct
  REGION 1..7: note = Engineered VH3 binding polypeptide
IAAKFDE

SEQ ID NO: 132 - protein, 7 aa, synthetic construct
  REGION 1..7: note = Engineered VH3 binding polypeptide
PAAKHDK

SEQ ID NO: 133 - protein, 7 aa, synthetic construct
  REGION 1..7: note = Engineered VH3 binding polypeptide
ADNAFNT

SEQ ID NO: 134 - protein, 7 aa, synthetic construct
  REGION 1..7: note = Engineered VH3 binding polypeptide
ADNRFNE

SEQ ID NO: 135 - protein, 7 aa, synthetic construct
  REGION 1..7: note = Engineered VH3 binding polypeptide
IDSKFDE

SEQ ID NO: 136 - protein, 7 aa, synthetic construct
  REGION 1..7: note = Engineered VH3 binding polypeptide
ADNRFNR

SEQ ID NO: 137 - protein, 7 aa, synthetic construct
  REGION 1..7: note = Engineered VH3 binding polypeptide
ADNKHNK

SEQ ID NO: 138 - protein, 7 aa, synthetic construct
  REGION 1..7: note = Engineered VH3 binding polypeptide
ADSKFDE

SEQ ID NO: 139 - protein, 7 aa, synthetic construct
  REGION 1..7: note = Engineered VH3 binding polypeptide
IDAKHDE

SEQ ID NO: 140 - protein, 7 aa, synthetic construct
  REGION 1..7: note = Engineered VH3 binding polypeptide
QQNKFNK

SEQ ID NO: 141 - protein, 7 aa, synthetic construct
  REGION 1..7: note = Engineered VH3 binding polypeptide
ADNKFHK

SEQ ID NO: 142 - protein, 4 aa, synthetic construct
  REGION 1..4: note = Engineered VH3 binding polypeptide
KFNK

SEQ ID NO: 143 - protein, 7 aa, synthetic construct
  REGION 1..7: note = Engineered VH3 binding polypeptide
ADNNFNR

SEQ ID NO: 144 - protein, 7 aa, synthetic construct
  REGION 1..7: note = Engineered VH3 binding polypeptide
AAAKHDK

SEQ ID NO: 145 - protein, 7 aa, synthetic construct
  REGION 1..7: note = Engineered VH3 binding polypeptide
IDNKFNK

SEQ ID NO: 146 - protein, 7 aa, synthetic construct
  REGION 1..7: note = Engineered VH3 binding polypeptide
IDAKFDE

SEQ ID NO: 147 - protein, 6 aa, synthetic construct
  REGION 1..6: note = Engineered VH3 binding polypeptide
DNNFNK

SEQ ID NO: 148 - protein, 7 aa, synthetic construct
  REGION 1..7: note = Engineered VH3 binding polypeptide
ADNKFNE

SEQ ID NO: 149 - protein, 7 aa, synthetic construct
  REGION 1..7: note = Engineered VH3 binding polypeptide
AAAQHDK

SEQ ID NO: 150 - protein, 7 aa, synthetic construct
  REGION 1..7: note = Engineered VH3 binding polypeptide
AAAKFDE

SEQ ID NO: 151 - protein, 4 aa, synthetic construct
  REGION 1..4: note = Engineered VH3 binding polypeptide
QAPK

SEQ ID NO: 152 - protein, 4 aa, synthetic construct
  REGION 1..4: note = Engineered VH3 binding polypeptide
QAPP

SEQ ID NO: 153 - protein, 4 aa, synthetic construct
  REGION 1..4: note = Engineered VH3 binding polypeptide
QAPR

SEQ ID NO: 154 - protein, 4 aa, synthetic construct
  REGION 1..4: note = Engineered VH3 binding polypeptide
QAPE

SEQ ID NO: 155 - protein, 4 aa, synthetic construct
  REGION 1..4: note = Engineered VH3 binding polypeptide
QAPG

SEQ ID NO: 156 - protein, 4 aa, synthetic construct
  REGION 1..4: note = Engineered VH3 binding polypeptide
QAIK

SEQ ID NO: 157 - protein, 58 aa, synthetic construct
  REGION 1..58: note = Engineered VH3 binding polypeptide
  VARIANT 8..11: note = misc_feature - Xaa can be any naturally occurring amino acid
  VARIANT 13..19: note = misc_feature - Xaa can be any naturally occurring amino acid
  VARIANT 23..26: note = misc_feature - Xaa can be any naturally occurring amino acid
  VARIANT 28..29: note = misc_feature - Xaa can be any naturally occurring amino acid
  VARIANT 33: note = misc_feature - Xaa can be any naturally occurring amino acid
  VARIANT 35..36: note = misc_feature - Xaa can be any naturally occurring amino acid
ADNKFNKXXXXAXXXXXXXPNLXXXXRXXFIQXLXXDPSQSANLLAEAKKLNDAQAPK

SEQ ID NO: 158 - protein, 58 aa, synthetic construct
  REGION 1..58: note = Engineered VH3 binding polypeptide
  VARIANT 8..11: note = misc_feature - Xaa can be any naturally occurring amino acid
  VARIANT 13..19: note = misc_feature - Xaa can be any naturally occurring amino acid
  VARIANT 23..26: note = misc_feature - Xaa can be any naturally occurring amino acid
  VARIANT 28..29: note = misc_feature - Xaa can be any naturally occurring amino acid
  VARIANT 33: note = misc_feature - Xaa can be any naturally occurring amino acid
  VARIANT 35..36: note = misc_feature - Xaa can be any naturally occurring amino acid
VDNKFNKXXXXAXXXXXXXPNLXXXXRXXFIQXLXXDPSQSANLLAEAKKLNDAQAPK

SEQ ID NO: 159 - protein, 58 aa, synthetic construct
  REGION 1..58: note = Engineered VH3 binding polypeptide
  VARIANT 8..11: note = misc_feature - Xaa can be any naturally occurring amino acid
  VARIANT 13..19: note = misc_feature - Xaa can be any naturally occurring amino acid
  VARIANT 23..26: note = misc_feature - Xaa can be any naturally occurring amino acid
  VARIANT 28..29: note = misc_feature - Xaa can be any naturally occurring amino acid
  VARIANT 33: note = misc_feature - Xaa can be any naturally occurring amino acid
  VARIANT 35..36: note = misc_feature - Xaa can be any naturally occurring amino acid
ADNKFNKXXXXAXXXXXXXPNLXXXXRXXFIQXLXXDPSVSKEILAEAKKLNDAQAPK

SEQ ID NO: 160 - protein, 58 aa, synthetic construct
  REGION 1..58: note = Engineered VH3 binding polypeptide
  VARIANT 8..11: note = misc_feature - Xaa can be any naturally occurring amino acid
  VARIANT 13..19: note = misc_feature - Xaa can be any naturally occurring amino acid
  VARIANT 23..26: note = misc_feature - Xaa can be any naturally occurring amino acid
  VARIANT 28..29: note = misc_feature - Xaa can be any naturally occurring amino acid
  VARIANT 33: note = misc_feature - Xaa can be any naturally occurring amino acid
  VARIANT 35..36: note = misc_feature - Xaa can be any naturally occurring amino acid
VDAKFDKXXXXAXXXXXXXPNLXXXXRXXFIQXLXXDPSQSANLLAEAKKLNDAQAPK

SEQ ID NO: 161 - protein, 58 aa, synthetic construct
  REGION 1..58: note = Engineered VH3 binding polypeptide
  VARIANT 8..11: note = misc_feature - Xaa can be any naturally occurring amino acid
  VARIANT 13..19: note = misc_feature - Xaa can be any naturally occurring amino acid
  VARIANT 23..26: note = misc_feature - Xaa can be any naturally occurring amino acid
  VARIANT 28..29: note = misc_feature - Xaa can be any naturally occurring amino acid
  VARIANT 33: note = misc_feature - Xaa can be any naturally occurring amino acid
  VARIANT 35..36: note = misc_feature - Xaa can be any naturally occurring amino acid
VDNKFNKXXXXAXXXXXXXPNLXXXXRXXFIQXLXXDPSVSKEILAEAKKLNDAQAPK

SEQ ID NO: 162 - protein, 58 aa, synthetic construct
  REGION 1..58: note = Engineered VH3 binding polypeptide
  VARIANT 8..11: note = misc_feature - Xaa can be any naturally occurring amino acid
  VARIANT 13..19: note = misc_feature - Xaa can be any naturally occurring amino acid
  VARIANT 23..26: note = misc_feature - Xaa can be any naturally occurring amino acid
  VARIANT 28..29: note = misc_feature - Xaa can be any naturally occurring amino acid
  VARIANT 33: note = misc_feature - Xaa can be any naturally occurring amino acid
  VARIANT 35..36: note = misc_feature - Xaa can be any naturally occurring amino acid
VDAKFDKXXXXAXXXXXXXPNLXXXXRXXFIQXLXXDPSVSKAILAEAKKLNDAQAPK

SEQ ID NO: 163 - protein, 58 aa, synthetic construct
  REGION 1..58: note = Engineered VH3 binding polypeptide
  VARIANT 8..11: note = misc_feature - Xaa can be any naturally occurring amino acid
  VARIANT 13..19: note = misc_feature - Xaa can be any naturally occurring amino acid
  VARIANT 23..26: note = misc_feature - Xaa can be any naturally occurring amino acid
  VARIANT 28..29: note = misc_feature - Xaa can be any naturally occurring amino acid
  VARIANT 33: note = misc_feature - Xaa can be any naturally occurring amino acid
  VARIANT 35..36: note = misc_feature - Xaa can be any naturally occurring amino acid
ADNNFNKXXXXAXXXXXXXPNLXXXXRXXFIQXLXXDPSQSANLLAEAKKLNESQAPK

SEQ ID NO: 164 - protein, 58 aa, synthetic construct
  REGION 1..58: note = Engineered VH3 binding polypeptide
  VARIANT 8..11: note = misc_feature - Xaa can be any naturally occurring amino acid
  VARIANT 13..19: note = misc_feature - Xaa can be any naturally occurring amino acid
  VARIANT 23..26: note = misc_feature - Xaa can be any naturally occurring amino acid
  VARIANT 28..29: note = misc_feature - Xaa can be any naturally occurring amino acid
  VARIANT 33: note = misc_feature - Xaa can be any naturally occurring amino acid
  VARIANT 35..36: note = misc_feature - Xaa can be any naturally occurring amino acid
ADNKHNKXXXXAXXXXXXXPNLXXXXRXXFIQXLXXDPSQSANLLAEAKKLNDAQAPK

SEQ ID NO: 165 - protein, 51 aa, synthetic construct
  REGION 1..51: note = Engineered VH3 binding polypeptide
  VARIANT 8..11: note = misc_feature - Xaa can be any naturally occurring amino acid
  VARIANT 13..19: note = misc_feature - Xaa can be any naturally occurring amino acid
  VARIANT 23..26: note = misc_feature - Xaa can be any naturally occurring amino acid
  VARIANT 28..29: note = misc_feature - Xaa can be any naturally occurring amino acid
  VARIANT 33: note = misc_feature - Xaa can be any naturally occurring amino acid
  VARIANT 35..36: note = misc_feature - Xaa can be any naturally occurring amino acid
ADNKFNKXXXXAXXXXXXXPNLXXXXRXXFIQXLXXDPSQSANLLAEAAPK

SEQ ID NO: 166 - protein, 58 aa, synthetic construct
  REGION 1..58: note = Engineered VH3 binding polypeptide
  VARIANT 8..11: note = misc_feature - Xaa can be any naturally occurring amino acid
  VARIANT 13..19: note = misc_feature - Xaa can be any naturally occurring amino acid
  VARIANT 23..26: note = misc_feature - Xaa can be any naturally occurring amino acid
  VARIANT 28..29: note = misc_feature - Xaa can be any naturally occurring amino acid
  VARIANT 33: note = misc_feature - Xaa can be any naturally occurring amino acid
  VARIANT 35..36: note = misc_feature - Xaa can be any naturally occurring amino acid
IAAQHDKXXXXAXXXXXXXPNLXXXXRXXFIQXLXXDPSVSLEILAEAKKLNDAQAPK

SEQ ID NO: 167 - protein, 58 aa, synthetic construct
  REGION 1..58: note = Engineered VH3 binding polypeptide
  VARIANT 8..11: note = misc_feature - Xaa can be any naturally occurring amino acid
  VARIANT 13..19: note = misc_feature - Xaa can be any naturally occurring amino acid
  VARIANT 23..26: note = misc_feature - Xaa can be any naturally occurring amino acid
  VARIANT 28..29: note = misc_feature - Xaa can be any naturally occurring amino acid
  VARIANT 33: note = misc_feature - Xaa can be any naturally occurring amino acid
  VARIANT 35..36: note = misc_feature - Xaa can be any naturally occurring amino acid
ADNKFHKXXXXAXXXXXXXPNLXXXXRXXFIQXLXXDPSQSANLLAEAKKLNDAQAPK

SEQ ID NO: 168 - protein, 54 aa, synthetic construct
  REGION 1..54: note = Engineered VH3 binding polypeptide
  VARIANT 4..7: note = misc_feature - Xaa can be any naturally occurring amino acid
  VARIANT 9..15: note = misc_feature - Xaa can be any naturally occurring amino acid
  VARIANT 19..22: note = misc_feature - Xaa can be any naturally occurring amino acid
  VARIANT 24..25: note = misc_feature - Xaa can be any naturally occurring amino acid
  VARIANT 29: note = misc_feature - Xaa can be any naturally occurring amino acid
  VARIANT 31..32: note = misc_feature - Xaa can be any naturally occurring amino acid
FNKXXXXAXXXXXXXPNLXXXXRXXFIQXLXXDPSQSANLLAEAKKLNDAQAPK

SEQ ID NO: 169 - protein, 58 aa, synthetic construct
  REGION 1..58: note = Engineered VH3 binding polypeptide
  VARIANT 8..11: note = misc_feature - Xaa can be any naturally occurring amino acid
  VARIANT 13..19: note = misc_feature - Xaa can be any naturally occurring amino acid
  VARIANT 23..26: note = misc_feature - Xaa can be any naturally occurring amino acid
  VARIANT 28..29: note = misc_feature - Xaa can be any naturally occurring amino acid
  VARIANT 33: note = misc_feature - Xaa can be any naturally occurring amino acid
  VARIANT 35..36: note = misc_feature - Xaa can be any naturally occurring amino acid
IAAQHDKXXXXAXXXXXXXPNLXXXXRXXFIQXLXXDPSVSLEILCEAKKLNDAQAPK

SEQ ID NO: 170 - protein, 58 aa, synthetic construct
  REGION 1..58: note = Engineered VH3 binding polypeptide
  VARIANT 8..11: note = misc_feature - Xaa can be any naturally occurring amino acid
  VARIANT 13..19: note = misc_feature - Xaa can be any naturally occurring amino acid
  VARIANT 23..26: note = misc_feature - Xaa can be any naturally occurring amino acid
  VARIANT 28..29: note = misc_feature - Xaa can be any naturally occurring amino acid
  VARIANT 33: note = misc_feature - Xaa can be any naturally occurring amino acid
  VARIANT 35..36: note = misc_feature - Xaa can be any naturally occurring amino acid
IDAKFDEXXXXAXXXXXXXPNLXXXXRXXFIQXLXXDPSVSLALLAEAKKLNDAQAPP

SEQ ID NO: 171 - protein, 57 aa, synthetic construct
  REGION 1..57: note = Engineered VH3 binding polypeptide
  VARIANT 7..10: note = misc_feature - Xaa can be any naturally occurring amino acid
  VARIANT 12..18: note = misc_feature - Xaa can be any naturally occurring amino acid
  VARIANT 22..25: note = misc_feature - Xaa can be any naturally occurring amino acid
  VARIANT 27..28: note = misc_feature - Xaa can be any naturally occurring amino acid
  VARIANT 32: note = misc_feature - Xaa can be any naturally occurring amino acid
  VARIANT 34..35: note = misc_feature - Xaa can be any naturally occurring amino acid
DNNFNKXXXXAXXXXXXXPNLXXXXRXXFIQXLXXDPSQSANLLAEAKKLNESQAPK

SEQ ID NO: 172 - protein, 58 aa, synthetic construct
  REGION 1..58: note = Engineered VH3 binding polypeptide
  VARIANT 8..11: note = misc_feature - Xaa can be any naturally occurring amino acid
  VARIANT 13..19: note = misc_feature - Xaa can be any naturally occurring amino acid
  VARIANT 23..26: note = misc_feature - Xaa can be any naturally occurring amino acid
  VARIANT 28..29: note = misc_feature - Xaa can be any naturally occurring amino acid
  VARIANT 33: note = misc_feature - Xaa can be any naturally occurring amino acid
  VARIANT 35..36: note = misc_feature - Xaa can be any naturally occurring amino acid
VDAKFDKXXXXAXXXXXXXPNLXXXXRXXFIQXLXXDPSQSANLLAEAKKLNDAQAIK

SEQ ID NO: 173 - protein, 58 aa, synthetic construct
  REGION 1..58: note = Engineered VH3 binding polypeptide
  VARIANT 8..11: note = misc_feature - Xaa can be any naturally occurring amino acid
  VARIANT 13..19: note = misc_feature - Xaa can be any naturally occurring amino acid
  VARIANT 23..26: note = misc_feature - Xaa can be any naturally occurring amino acid
  VARIANT 28..29: note = misc_feature - Xaa can be any naturally occurring amino acid
  VARIANT 33: note = misc_feature - Xaa can be any naturally occurring amino acid
  VARIANT 35..36: note = misc_feature - Xaa can be any naturally occurring amino acid
ADNRFNEXXXXAXXXXXXXPNLXXXXRXXFIQXLXXDPSVSKEILAEAKKLNDAQAPE

SEQ ID NO: 174 - protein, 58 aa, synthetic construct
  REGION 1..58: note = Engineered VH3 binding polypeptide
  VARIANT 8..11: note = misc_feature - Xaa can be any naturally occurring amino acid
  VARIANT 13..19: note = misc_feature - Xaa can be any naturally occurring amino acid
  VARIANT 23..26: note = misc_feature - Xaa can be any naturally occurring amino acid
  VARIANT 28..29: note = misc_feature - Xaa can be any naturally occurring amino acid
  VARIANT 33: note = misc_feature - Xaa can be any naturally occurring amino acid
  VARIANT 35..36: note = misc_feature - Xaa can be any naturally occurring amino acid
IDAKFDEXXXXAXXXXXXXPNLXXXXRXXFIQXLXXDPSVSLSLLAEAKKLNDAQAPP

SEQ ID NO: 175 - protein, 251 aa, synthetic construct
  REGION 1..251: note = Engineered VH3 binding polypeptide
AQYEDGKQYTDTVDAKFDKEAYRALAEIARLPNLTEEQRAAFIQSLKDDPSVSKAILAEAKKLNDAQAPKVDAKFDKEAYRALAEIARLPNLTEEQRAAFIQSLKDDPSVSKAILAEAKKLNDAQAPKVDAKFDKEAYRALAEIARLPNLTEEQRAAFIQSLKDDPSVSKAILAEAKKLNDAQAPKVDAKFDKEAYRALAEIARLPNLTEEQRAAFIQSLKDDPSVSKAILAEAKKLNDAQAPKHHHHHHC

SEQ ID NO: 176 - protein, 251 aa, synthetic construct
  REGION 1..251: note = Engineered VH3 binding polypeptide
AQYEDGKQYTDTVDAKFDKEAYRALAEIARLPNLTEEQRAGFIQSLKDDPSVSKAILAEAKKLNDAQAPKVDAKFDKEAYRALAEIARLPNLTEEQRAGFIQSLKDDPSVSKAILAEAKKLNDAQAPKVDAKFDKEAYRALAEIARLPNLTEEQRAGFIQSLKDDPSVSKAILAEAKKLNDAQAPKVDAKFDKEAYRALAEIARLPNLTEEQRAGFIQSLKDDPSVSKAILAEAKKLNDAQAPKHHHHHHC

SEQ ID NO: 177 - protein, 367 aa, synthetic construct
  REGION 1..367: note = Engineered VH3 binding polypeptide
AQYEDGKQYTDTVDAKFDKEAYRALAEIARLPNLTEEQRAGFIQSLKDDPSVSKAILAEAKKLNDAQAPKVDAKFDKEAYRALAEIARLPNLTEEQRAGFIQSLKDDPSVSKAILAEAKKLNDAQAPKVDAKFDKEAYRALAEIARLPNLTEEQRAGFIQSLKDDPSVSKAILAEAKKLNDAQAPKVDAKFDKEAYRALAEIARLPNLTEEQRAGFIQSLKDDPSVSKAILAEAKKLNDAQAPKVDAKFDKEAYRALAEIARLPNLTEEQRAGFIQSLKDDPSVSKAILAEAKKLNDAQAPKVDAKFDKEAYRALAEIARLPNLTEEQRAGFIQSLKDDPSVSKAILAEAKKLNDAQAPKHHHHHHC

SEQ ID NO: 178 - protein, 450 aa, synthetic construct
  REGION 1..450: note = Humanized mus musculus antibody fragment
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 214 aa, synthetic construct
  REGION 1..214: note = Humanized mus musculus antibody fragment
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - protein, 58 aa, synthetic construct
  REGION 1..58: note = Engineered HER2 binding polypeptide
VDNKFNKEMRNAYWEIALLPNLNHQQKRAFIRSLYDDPSQSANLLAEAKKLNDAQAPK

SEQ ID NO: 181 - protein, 361 aa, synthetic construct
  REGION 1..361: note = Engineered VH3 binding polypeptide
AQYEDGKQYTGTVDAKFDKEAQEAFYEILHLPNLTEEQRNAFIQSLKDDPSVSKAILAEAKKLNDAQAPKVDAKFDKEAQEAFYEILHLPNLTEEQRNAFIQSLKDDPSVSKAILAEAKKLNDAQAPKVDAKFDKEAQEAFYEILHLPNLTEEQRNAFIQSLKDDPSVSKAILAEAKKLNDAQAPKVDAKFDKEAQEAFYEILHLPNLTEEQRNAFIQSLKDDPSVSKAILAEAKKLNDAQAPKVDAKFDKEAQEAFYEILHLPNLTEEQRNAFIQSLKDDPSVSKAILAEAKKLNDAQAPKVDAKFDKEAQEAFYEILHLPNLTEEQRNAFIQSLKDDPSVSKAILAEAKKLNDAQAPKC

SEQ ID NO: 182 - protein, 237 aa, synthetic construct
  REGION 1..237: note = Humanized mus musculus antibody fragment
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEL

SEQ ID NO: 183 - protein, 214 aa, synthetic construct; identical to SEQ ID NO: 179
  REGION 1..214: note = Humanized mus musculus antibody fragment

SEQ ID NO: 184 - protein, 213 aa, synthetic construct
  REGION 1..213: note = Humanized mus musculus antibody fragment
LGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 58 aa, synthetic construct [sequence omitted]

SEQ ID NO: 186 - protein, 58 aa, synthetic construct
IAAQHDKDQQAAFYEILHLPNLTEEQRNAFIQSLRDDPSVSLEILAEAKKLNDAQAPK

SEQ ID NO: 187 - protein, 58 aa, synthetic construct
IAAQHDKEHQAAFYEILHLPNLTEDQRNAFIQSLRDDPSVSLEILGEAKKLNDAQAPK

SEQ ID NO: 188 - protein, 58 aa, synthetic construct
IAAQHDKDQQAAFYEILHLPNLTEEQRNAFIQSLRHDPSVSLEILAEAKKLNDAQAPK

SEQ ID NO: 189 - protein, 58 aa, synthetic construct
ADNKFNKEQQNAFYEILHLPNLTEEQRNAFIQSLRDEPSVSKEILAEAKKLNDAQAPK

SEQ ID NO: 190 - protein, 58 aa, synthetic construct
ADSKFDEAQQSAFYEILHLPSLTEDQRAAFIQSLKDDPSTSKSLLGEAKKLNDAQAPP

SEQ ID NO: 191 - protein, 58 aa, synthetic construct
VDNKFNKEQQNAFYEILHLPNLTEEQRNAFIQSLKDDPSVSKEILKEAKKLNDAQAPK

SEQ ID NO: 192 - protein, 58 aa, synthetic construct
VDAKFNKEQQNAFYEILNLPNLTEEQRNAFIQSLRDDPSQSANLLSEAKRLNESQAPG

SEQ ID NO: 193 - protein, 58 aa, synthetic construct
VDAKFNKEQQNAFYEILHLPNLTEEQRNAFIQSLRDDPSTSKSILKEAKKLNKAQAPK

SEQ ID NO: 194 - protein, 56 aa, synthetic construct
AQHDEAAYRALAQVARMPNLNADQRAGFIQSLKDDPSQSANVLGEAQKLNDSQAPK

SEQ ID NO: 195 - protein, 61 aa, synthetic construct; identical to SEQ ID NO: 86

SEQ ID NO: 196 - protein, 58 aa, synthetic construct
ADNNFNKEAYRALAEIARMPNLNEEQRAGFIQSLKDDPSQSANLLAEAKKLNESQAPK

SEQ ID NO: 197 - protein, 58 aa, synthetic construct; identical to SEQ ID NO: 84

SEQ ID NO: 198 - protein, 58 aa, synthetic construct; identical to SEQ ID NO: 83

SEQ ID NO: 199 - protein, 58 aa, synthetic construct; identical to SEQ ID NO: 56

SEQ ID NO: 200 - protein, 58 aa, synthetic construct
VDAKFDKEAYRALAEIARLPNLTEEQRAGFIQSLKDDPSQSANLLAEAKKLNDAQAPK

SEQ ID NO: 201 - protein, 58 aa, synthetic construct
IAAQHDKDAYRALAEIARLPNLTEEQRAGFIQSLRDDPSVSLEILAEAKKLNDAQAPK

SEQ ID NO: 202 - protein, 58 aa, synthetic construct
IAAQHDKEAYRALAEIARLPNLTEDQRAGFIQSLRDDPSVSLEILGEAKKLNDAQAPK

SEQ ID NO: 203 - protein, 58 aa, synthetic construct
IAAQHDKDAYRALAEIARLPNLTEEQRAGFIQSLRHDPSVSLEILAEAKKLNDAQAPK

SEQ ID NO: 204 - protein, 58 aa, synthetic construct
ADNKFNKEAYRALAEIARLPNLTEEQRAGFIQSLRDEPSVSKEILAEAKKLNDAQAPK

SEQ ID NO: 205 - protein, 58 aa, synthetic construct
ADSKFDEAAYRALAEIARLPSLTEDQRAGFIQSLKDDPSTSKSLLGEAKKLNDAQAPP

SEQ ID NO: 206 - protein, 58 aa, synthetic construct
VDNKFNKEAYRALAEIARLPNLTEEQRAGFIQSLKDDPSVSKEILKEAKKLNDAQAPK

SEQ ID NO: 207 - protein, 58 aa, synthetic construct
VDAKFNKEAYRALAEIARLPNLTEEQRAGFIQSLRDDPSQSANLLSEAKRLNESQAPG

SEQ ID NO: 208 - protein, 58 aa, synthetic construct
VDAKFNKEAYRALAEIARLPNLTEEQRAGFIQSLRDDPSTSKSILKEAKKLNKAQAPK

SEQ ID NO: 209 - protein, 58 aa, synthetic construct
ADNNFNKEAYRALAEIARMPNLNEESRAGFIQSLKDDPSQSANLLAEAKKLNESQAPK

SEQ ID NO: 210 - protein, 58 aa, synthetic construct
ADNKFNKEAYRALAEIARLPNLNEESRAGFIQSLKDDPSQSANLLAEAKKLNDAQAPK

SEQ ID NO: 211 - protein, 58 aa, synthetic construct
ADNKFNKEAYRALAEIARLPNLTEESRAGFIQSLKDDPSVSKEILAEAKKLNDAQAPK

SEQ ID NO: 212 - protein, 58 aa, synthetic construct
VDNKFNKEAYRALAEIARLPNLNEESRAGFIQSLKDDPSQSANLLAEAKKLNDAQAPK

SEQ ID NO: 213 - protein, 58 aa, synthetic construct
VDAKFDKEAYRALAEIARLPNLTEESRAGFIQSLKDDPSQSANLLAEAKKLNDAQAPK

SEQ ID NO: 214 - protein, 58 aa, synthetic construct
IAAQHDKDAYRALAEIARLPNLTEESRAGFIQSLRDDPSVSLEILAEAKKLNDAQAPK

SEQ ID NO: 215 - protein, 58 aa, synthetic construct
IAAQHDKEAYRALAEIARLPNLTEDSRAGFIQSLRDDPSVSLEILGEAKKLNDAQAPK

SEQ ID NO: 216 - protein, 58 aa, synthetic construct
IAAQHDKDAYRALAEIARLPNLTEESRAGFIQSLRHDPSVSLEILAEAKKLNDAQAPK

SEQ ID NO: 217 - protein, 58 aa, synthetic construct
ADNKFNKEAYRALAEIARLPNLTEESRAGFIQSLRDEPSVSKEILAEAKKLNDAQAPK

SEQ ID NO: 218 - protein, 58 aa, synthetic construct
ADSKFDEAAYRALAEIARLPSLTEDSRAGFIQSLKDDPSTSKSLLGEAKKLNDAQAPP

SEQ ID NO: 219 - protein, 58 aa, synthetic construct
VDNKFNKEAYRALAEIARLPNLTEESRAGFIQSLKDDPSVSKEILKEAKKLNDAQAPK

SEQ ID NO: 220 - protein, 58 aa, synthetic construct
VDAKFNKEAYRALAEIARLPNLTEESRAGFIQSLRDDPSQSANLLSEAKRLNESQAPG

SEQ ID NO: 221 - protein, 58 aa, synthetic construct
VDAKFNKEAYRALAEIARLPNLTEESRAGFIQSLRDDPSTSKSILKEAKKLNKAQAPK

SEQ ID NO: 222 - protein, 56 aa, synthetic construct
AQHDEAAYEARAQVARMPNLNADQRAGFIQSLKDDPSQSANVLGEAQKLNDSQAPK

SEQ ID NO: 223 - protein, 58 aa, synthetic construct
ADNKFNKEAYEARAEIARLPNLNEEQRAGFIQSLKDDPSQSANLLAEAKKLNDAQAPK

SEQ ID NO: 224 - protein, 58 aa, synthetic construct
ADNKFNKEAYEARAEIARLPNLTEEQRAGFIQSLKDDPSVSKEILAEAKKLNDAQAPK

SEQ ID NO: 225 - protein, 56 aa, synthetic construct
AQHDEAAYEARAQVALMPNLNADQRAGFIQSLKDDPSQSANVLGEAQKLNDSQAPK

SEQ ID NO: 226 - protein, 58 aa, synthetic construct
ADNKFNKEAYEARAEIALLPNLNEEQRAGFIQSLKDDPSQSANLLAEAKKLNDAQAPK

SEQ ID NO: 227 - protein, 58 aa, synthetic construct
ADNKFNKEAYEARAEIALLPNLTEEQRAGFIQSLKDDPSVSKEILAEAKKLNDAQAPK

SEQ ID NO: 228 - protein, 56 aa, synthetic construct
AQHDEAAQRARAQVLRMPNLNADQRAGFIQSLKDDPSQSANVLGEAQKLNDSQAPK

SEQ ID NO: 229 - protein, 58 aa, synthetic construct
ADNKFNKEAQRARAEILRLPNLNEEQRAGFIQSLKDDPSQSANLLAEAKKLNDAQAPK

SEQ ID NO: 230 - protein, 58 aa, synthetic construct
ADNKFNKEAQRARAEILRLPNLTEEQRAGFIQSLKDDPSVSKEILAEAKKLNDAQAPK

SEQ ID NO: 231 - protein, 56 aa, synthetic construct
AQHDEAAYEALRQVARMPNLNADQRAGFIQSLKDDPSQSANVLGEAQKLNDSQAPK

SEQ ID NO: 232 - protein, 58 aa, synthetic construct
ADNKFNKEAYEALREIARLPNLNEEQRAGFIQSLKDDPSQSANLLAEAKKLNDAQAPK

SEQ ID NO: 233 - protein, 58 aa, synthetic construct
ADNKFNKEAYEALREIARLPNLTEEQRAGFIQSLKDDPSVSKEILAEAKKLNDAQAPK

SEQ ID NO: 234 - protein, 56 aa, synthetic construct
AQHDEAAYEAARQVARMPNLNADQRAGFIQSLKDDPSQSANVLGEAQKLNDSQAPK

SEQ ID NO: 235 - protein, 58 aa, synthetic construct
ADNKFNKEAYEAAREIARLPNLNEEQRAGFIQSLKDDPSQSANLLAEAKKLNDAQAPK

SEQ ID NO: 236 - protein, 58 aa, synthetic construct
ADNKFNKEAYEAAREIARLPNLTEEQRAGFIQSLKDDPSVSKEILAEAKKLNDAQAPK

SEQ ID NO: 237 - protein, 56 aa, synthetic construct
AQHDEAYQRALAQVALMPNLNADQRAGFIQSLKDDPSQSANVLGEAQKLNDSQAPK

SEQ ID NO: 238 - protein, 58 aa, synthetic construct
ADNKFNKEYQRALAEIALLPNLNEEQRAGFIQSLKDDPSQSANLLAEAKKLNDAQAPK

SEQ ID NO: 239 - protein, 58 aa, synthetic construct
ADNKFNKEYQRALAEIALLPNLTEEQRAGFIQSLKDDPSVSKEILAEAKKLNDAQAPK